(19) United States
(12) Patent Application Publication
(10) Pub. No.: US 2022/0155308 A1
(43) Pub. Date: May 19, 2022

(54) LYOPHILIZED ANTIBODY PANEL

(71) Applicant: Fluidigm Canada Inc., Markham (CA)

(72) Inventors: Stephen K.H. Li, Thornhill (CA); Daniel Majonis, North York (CA); Vladimir Baranov, Richmond Hill (CA); Olga Ornatsky, Richmond Hill (CA); Bedilu Allo, Toronto (CA); Dmitry Bandura, Aurora (CA); Colin Thom, Stanford, CA (US); Greg Stelzer, Thompson?s Station, TN (US); Alexander Loboda, Thornhill (CA)

(73) Assignee: Fluidigm Canada Inc., Markham, ON (CA)

(21) Appl. No.: 17/439,503

(22) PCT Filed: Mar. 27, 2020
(86) PCT No.: PCT/US2020/025296

§ 371 Date: Sep. 15, 2021

Related U.S. Application Data
(60) Provisional application No. 62/824,917, filed on Mar. 27, 2019.
(60) Provisional application No. 62/951,546, filed on Dec. 20, 2019.

(51) Int. Cl. G01N 33/58; G01N 1/30; G01N 15/10

(52) U.S. Cl. CPC G01N 33/58; G01N 1/30; G01N 2015/1006; G01N 2001/302; G01N 15/10

(57) ABSTRACT

A lyophilized antibody panel is disclosed for interrogation using elemental analysis. The antibody panel includes multiple antibodies each element-tagged or element-labelled with one or more isotopes such that each different antibody is isotopically distinguishable from the other antibodies. Each element tag can include one or more unique isotopes or unique combinations of isotopes. The set of element-tagged antibodies can be lyophilized in admixture. Thus, the lyophilized element-tagged antibody panel can be easily and efficiently resuspended and mixed with a sample prior to interrogation with an elemental analyzer, such as a mass spectrometer. This lyophilized element-tagged antibody panel can provide the benefits of an element-tagged assay while also being easy to use and remaining stable for long durations.

### CROSS-REFERENCE TO RELATED APPLICATIONS

This application claims the benefit of priority to U.S. Provisional Application No. 62/824,917, filed Mar. 27, 2019 and U.S. Provisional Application No. 62/951,546, filed Dec. 20, 2019, the contents of both of which are incorporated herein by reference for all purposes.

### TECHNICAL FIELD

The present disclosure relates to biological assays generally and more specifically to biological assays interrogated through elemental analysis.

### BACKGROUND

Biological assays can be used to determine information associated with a sample. For example, certain biological assays can be conducted to determine the presence of different cell types in a sample of blood. The results of a biological assay can have uses across research and medicine.

In certain current assays, various antibodies are tagged with different fluorescent materials. Each antibody can be specific to a particular protein (e.g., antigen) that may or may not be present in a sample. When these fluorescent-tagged antibodies are mixed with the sample, antibodies specific to proteins in the sample can bind to those proteins. Then, after washing the sample, the resultant sample can be optically interrogated to identify the presence of the fluorescent materials.

However, such fluorescent assays are limited in the number of distinguishable channels and the time required to conduct the assay. For example, the multiplexity of traditional fluorescence microscopy is limited by the spectral overlap of fluorophore emissions. The recent development of imaging mass spectrometry of samples stained with element labelled antibodies allows for many more targets to be imaged simultaneously, as each target can be associated with a unique isotope through an antibody intermediate. However, traditional techniques for creating and storing element-labelled assays can be susceptible to damage and other error-inducing effects, which can result in inaccurate results.

### BRIEF DESCRIPTION OF THE DRAWINGS

The specification makes reference to the following appended figures, in which use of like reference numerals in different figures is intended to illustrate like or analogous components.

FIG. 1 is a schematic diagram of an element-tagged moiety according to certain aspects of the present disclosure.

FIG. 2A is a schematic diagram of a barcoding reagent with a separate sample barcode according to certain aspects of the present disclosure.

FIG. 2B is a schematic diagram of the barcoding reagent of FIG. 2A after attachment of the sample barcode according to certain aspects of the present disclosure.

FIG. 3 is a schematic diagram of a barcoding reagent 308 with integrated sample barcode 312 according to certain aspects of the present disclosure.

FIG. 4 is a schematic diagram of a system for assaying one or more samples using an elemental analyzer and a lyophilized antibody panel according to certain aspects of the present disclosure.

FIG. 5 is a schematic diagram depicting the analysis and processing of unknown particles according to certain aspects of the present disclosure.

FIG. 6 is a schematic diagram depicting three example element tags with charts illustrating their respective elemental data according to certain aspects of the present disclosure.

FIG. 7 is a schematic diagram of a sample cell tagged with an element-tagged moiety, a sample barcode, and a barcoding reagent according to certain aspects of the present disclosure.

FIG. 8 is a schematic diagram depicting a sample cell tagged with a multi-isotope sample barcode according to certain aspects of the present disclosure.

FIG. 9 is a schematic diagram depicting a sample cell tagged with distributed sample barcode parts according to certain aspects of the present disclosure.

FIG. 10 is a schematic diagram of a barcoding reagent and reporter antibody according to certain aspects of the present disclosure.

FIG. 11 is a schematic diagram depicting the preparation of a lyophilized antibody panel according to certain aspects of the present disclosure.

FIG. 12 is a schematic diagram of a process for preparing barcoding reagents according to certain aspects of the present disclosure.

FIG. 13 is a flowchart depicting a process for staining and analyzing a blood sample according to certain aspects of the present disclosure.

FIG. 14 is a schematic diagram depicting an example gating strategy for automatically analyzing elemental data according to certain aspects of the present disclosure.

FIG. 15 is a schematic diagram depicting a technique for sample-labeling barcoding reagents and analyzing a set of samples according to certain aspects of the present disclosure.

FIG. 16 is a schematic diagram depicting a technique for sample-labeling and analyzing a set of samples according to certain aspects of the present disclosure.

FIG. 17 is a schematic diagram depicting preparing a set of pre-configured sample-barcode-labeled barcoding reagents according to certain aspects of the present disclosure.

### DETAILED DESCRIPTION

Certain aspects and features of the present disclosure relate to a lyophilized antibody panel for interrogation using elemental analysis. An antibody may be a fragment thereof, such as a nanobody or Fab fragment. Further, affinity reagents in general (including not just antibodies but other affinity reagents such as lectins, aptamers and non-antibody protein-ligand pairs such as streptavidin-biotin) may be used in place of antibodies in the embodiments discussed herein.

The antibody panel can include multiple different antibodies that are each element-tagged or element-labelled with one or more isotopes such that each different antibody is isotopically distinguishable from the other antibodies. Each element tag can include one or more unique elements, isotopes or unique combinations of isotopes. The set of element-tagged antibodies can be lyophilized in admixture. Thus, the lyophilized element-tagged antibody panel can be easily and efficiently resuspended and mixed with a sample prior to interrogation with an elemental analyzer, such as a mass spectrometer. This lyophilized element-tagged antibody panel can provide the benefits of an element-tagged assay while also being easy to use and remaining stable for long durations. Element tags of any of the subject methods or kits may be mass tags, such as when the method of detection is mass spectrometry and/or when the element tag comprises an enriched isotope. The same mass tag, or overlapping mass tags, refer to mass tags that have the same isotope (or isotope mass) labeling atom, and would be indistinguishable by mass spectrometry.

Certain aspects and features of the present disclosure also relate to techniques for uniquely barcoding different samples, permitting the samples to be combined prior to interrogation by an elemental analyzer. Different sample barcoding reagents can each include a unique combination of isotopes usable to distinguish that sample barcoding reagent from other sample barcoding reagents. Multiple samples can be individually mixed with the different sample barcoding reagents, permitting the sample barcoding reagents to bind to targets (e.g., assay barcoded beads and/or cells) in the samples, thus labelling each sample with its own, unique barcode. Even after mixing the samples together, the data for each sample can be extracted based on the presence of the unique combinations of isotopes in the data collected form an elemental analyzer. In some cases, the samples can even be mixed together prior to conducting an assay, such as an assay using a lyophilized element-tagged antibody panel.

Elemental analysis is a process in which a sample is analyzed for its elemental composition and sometimes isotopic composition. Elemental analysis can be accomplished by a number of methods, including: optical atomic spectroscopy, such as flame atomic absorption, graphite furnace atomic absorption, and inductively coupled plasma atomic emission, which probe the outer electronic structure of atoms; mass spectrometric atomic spectroscopy, such as inductively coupled mass spectrometry, which probes the mass of atoms; X-ray fluorescence, particle induced x-ray emission, x-ray photoelectron spectroscopy, and Auger electron spectroscopy which probes the inner electronic structure of atoms. Other elemental analysis techniques may be used.

Mass spectrometers for use in the invention may be selected based on the needs of the operator or specific application. Example types of mass spectrometers include quadrupole, time of flight, magnetic sector, high resolution, single or multicollector mass spectrometers. Typically, time of flight or magnetic sector mass spectrometers are used for the recording of fast transient events with the transit durations that are expected from particles (e.g., bead, cell or laser ablation plume). In certain aspects, a time-of-flight mass spectrometer may have a high pass filter, such as with a mass cutoff of 80 amu or higher. In certain aspects, atomization and ionization source(s), such as an inductively coupled plasma (ICP), may be upstream of a time-of-flight or magnetic sector mass detector.

Certain aspects of the present disclosure are especially suited for use with a type of elemental analysis called mass analysis, wherein the mass of the isotope or element is determined. Mass analysis can include atomic mass analysis, such as mass cytometry. Mass analysis can be especially suitable for identifying an element's isotope, and thus differentiating one isotope from another distinct isotope or differentiating one combination of isotopes from another distinct combination of isotopes. Therefore, mass analysis can be a desired form of elemental analysis for all aspects of the present disclosure disclosed herein, where appropriate.

An elemental analyzer is an instrument for the quantitation of the atomic composition of a sample. An elemental analyzer can employ one of the elemental analysis techniques described herein. An elemental analyzer can have a set number of channels for detection of or differentiation of isotopes. For example, a mass spectrometer can have a number of mass channels usable for detection of different isotopes. The number of channels available to a particular elemental analyzer can be limited. Thus, the use of any subset of channels for a particular purpose (e.g., labelling antibodies of a lyophilized antibody panel or barcoding samples) can leave remaining channels available for other purposes. For example, empty channels can be used to perform additional assays or other barcoding.

In some cases, elemental analysis can be conducted on an individual particle basis, known as particle elemental analysis. Particle elemental analysis includes determining the elemental composition of individual particles (e.g., cell-by-cell), such as using a mass spectrometer-based flow cytometer. Certain aspects of the present disclosure make use of particle elemental analysis on a cell-by-cell basis, which can be known as cytometric elemental analysis. In some cases, elemental analysis can be conducted on a bulk basis, known as bulk elemental analysis or solution elemental analysis. Bulk elemental analysis includes determining the elemental composition of the entire volume of a sample.

Elemental analysis can be used to interrogate a sample, such as a biological sample. If the sample is labelled with a known element tag, detection of the element tag during elemental analysis can be indicative of characteristics of the sample associated with the element tag.

As referred to herein, mass cytometry is any method of detecting element tags (mass tags) in a biological sample, such as simultaneously detecting a plurality of distinguishable mass tags with single cell resolution. Mass cytometry may include analysis of mass tagged beads, separate from or in addition to cells. Any of the subject kits and methods may include or be adapted to mass cytometry. Mass cytometry includes suspension mass cytometry and imaging mass cytometry (IMC).

Suspension mass cytometry includes analysis of suspended element tagged cells and/or beads by mass spectrometry (e.g., by atomic mass spectrometry), and is described in US patent publications including US20050218319, US20150183895, US20150122991, all of which are incorporated by reference herein.

Imaging mass cytometry includes any imaging mass spectrometry (e.g., imaging atomic mass spectrometry) of element tagged biological sample, such as a tissue section or cell smear. IMC may atomize and ionize mass tags of a cellular sample by one or more of laser radiation, ion beam radiation, electron beam radiation, and/or inductively coupled plasma (ICP). Mass cytometry may simultaneously detect distinct mass tags from single cells, such as by time of flight (TOF) or magnetic sector mass spectrometry (MS). Examples of mass cytometry include suspension mass cytometry where cells are flowed into and ICP-MS and imaging mass cytometry where a cellular sample (e.g., tissue section) is sampled, for example by laser ablation (LA-ICP-MS) or by a primary ion beam (e.g., for SIMS). Laser based IMC is described in US patent publications US20160131635, US20170148619, US20180306695, and US20180306695 all of which are incorporated by reference herein. In certain aspects, when the sample is a cell smear for analysis by IMC, the cells may be processed as described herein such as by staining with a lyophilized panel, sample barcoding, and/or assay barcoding. Similarly, assay beads described herein may be analyzed separately or in mixture with cells, by IMC.

Mass tags may be sampled, atomized and ionized prior to elemental analysis. For example, mass tags in a biological sample may be sampled, atomized and/or ionized by radiation such as a laser beam, ion beam or electron beam. Alternatively or in addition, mass tags may be atomized and ionized by a plasma, such as an inductively coupled plasma (ICP). In suspension mass cytometry, whole cells including mass tags may be flowed into an ICP-MS, such as an ICP-TOF-MS. In imaging mass cytometry, a form of radiation may remove (and optionally ionize and atomize) portion (e.g., pixels, region of interest) of a solid biological sample, such as a tissue sample, including mass tags. Examples of IMC include LA-ICP-MS and SIMS-MS of mass tagged sample. In certain aspects, ion optics may deplete ions other than the isotope of the mass tags. For example, ion optics may remove lighter ions (e.g., C, N, O), organic molecular ions. In ICP applications, ion optics may remove gas such as Ar and/or Xe, such as through a high-pass quadrupole filter. In certain aspects, IMC may provide an image of mass tags (e.g., targets associated with mass tags) with cellular or subcellular resolution.

Similar to fluorescent immunohistochemistry methods, mass cytometry (including imaging mass cytometry) workflows may include cell (e.g., tissue) fixation and/or permeabilization prior to staining with antibodies and/or other specific binding partners. In contrast to fluorescent methods, in mass cytometry mass tags (e.g., comprising heavy metals not endogenous to the cell) are associated with target analytes through specific binding partners such as antibodies. Imaging mass cytometry, like fluorescent microscopy, may include an antigen retrieval step where the sample is exposed to conditions such as heat to expose target analytes for binding by biomolecules. Unbound biomolecules are typically washed off before detection of mass tags by mass spectrometry. Of note, other methods of detection such as elemental analysis (e.g., emission spectroscopy or X-ray dispersion spectroscopy) are also within the scope of the subject application.

Of note, antigen retrieval conditions may be particularly important for IMC over other imaging methods, as the element tag (e.g., mass tag) may create more steric hindrance in tissue than other labels such as fluorophores. However, highly stringent retrieval conditions (such as prolonged exposure of a tissue section to high heat) may denature the sample, damaging the epitopes to be detected. In certain aspects, a metal heating block, such as an aluminum heating block, may be provided in a kit or used to heat a tissue section for antigen retrieval. Inventors have found that such a block provides even heat distribution and allows suitably quick temperature transition for controlled antigen retrieval. As such, a method or kit of the subject application, such as for any imaging mass cytometry application or another imaging application such as light microscopy.

Additional reagents for mass cytometry include metal-containing biosensor(s) (e.g., that is deposited or bound under conditions such as hypoxia, protein synthesis, cell cycle and/or cell death) and/or metal containing histochemical compound(s) that bind to structures (e.g., DNA, cell membrane, strata) based on chemical properties. In addition, mass tags (e.g., of the subject application or other mass tags) may be combined to provide a unique barcode, so as to label a particular sample or experimental condition prior to pooling with other samples or experimental conditions.

In IMC, a tissue sample may be a section e.g. having a thickness within the range of 1-10 μm, such as between 2-6 μm may be used. In some cases, an ultrathin section less than 500 nm, 400 nm, 200 nm, 100 nm or 50 nm thick may be used, such as sample cut from a resin-embedded tissue block. Techniques for preparing such sections are well known from the field of IHC e.g. using microtomes, including dehydration steps, fixation, embedding, permeabilization, sectioning etc. Thus, a tissue may be chemically fixed and then sections can be prepared in the desired plane. Cryosectioning or laser capture microdissection can also be used for preparing tissue samples. Samples may be permeabilized e.g. to permit of reagents for labelling of intracellular targets. Even after antigen retrieval (e.g., by heating), access to an analyte by an biomolecule may be sterically hindered. As such, smaller biomolecules and certain mass tags may best allow for the biomolecule to access its target analyte.

Cell segmentation in imaging mass cytometry may allow the leveraging of automated cell classification and other aspects of the subject application. However, different tissue and cell type have different surface markers, making a single universal membrane stain difficult.

A kit for cell segmentation may include a membrane stain comprising a plurality of antibodies to different cell surface targets, wherein the antibodies are conjugated to the same mass tag. In certain aspects, a membrane stain comprises an antibody to a junction protein and an antibody to a non-junction protein.

The membrane stain does not comprise antibodies that stains targets in compartments other than the plasma membrane. The membrane stain may bind to membranes of more cell types than any individual antibody of the membrane stain. The plurality of antibodies may be in mixture.

A kit may further include a nuclear stain and/or a cytosol stain, e.g., for better individual identifying cells and guiding cell segmentation along the membrane. The kit further comprises a panel of antibodies to cell surface targets, wherein antibodies of the panel are conjugated to different mass tags and useful for identifying cell populations as described herein (e.g., by automate classification of populations).

In certain aspects, the membrane stain may apply before, alongside, or after other antibody panels. Cell segmentation may be performed on images obtained by imaging mass cytometry. Segmentation approaches are known in the art, and are described, for example, by Wang et al. in “Cell Segmentation for Image Cytometry: Advances, Insufficiencies, and Challenges.” Cytometry. Part A (2019): 708-711. In general, cell segmentation benefits from clear labeling of cell membrane and of the interior of the cell (e.g., a cell nucleus visualized by a nuclear stain).

Information on protein localization is centralized in the Human Protein Atlas, which is searchable to identify protein targets that specifically localize to the plasma membrane (e.g., cell junctions) across different tissue and cell types. A complementary set of protein targets that identify cell membrane within and across a number of tissues can thus be identified, and proteins to those targets can be tagged with the same element tag to provide a membrane stain of the subject application. The membrane stain may include 1, 2, 3, 4 or more antibodies that specifically bind to Syntaxin 4, Solute carrier family 16 member 1, Erythrocyte membrane protein band 4.1 like 3, Adaptor related protein complex 2 mu 1 subunit, G protein subunit beta 2, Moesin, EZR, CTNNB1, ATPase Na+/K+ transporting subunit beta 3, Phosphatidylethanolamine binding protein 1, Catenin beta 1, Catenin beta 1, Solute carrier family 1 member 5, Ezrin, 5100 calcium binding protein A4, and Ankyrin 3, which are reported as well expressed membrane proteins conserved across different cell lines. Alternatively or in addition, the membrane stain may include 1, 2, 3, 4 or more antibodies that specifically bind to CDH17, CTNNA1, DNAJC18, GJB6, TJP3, and C4 of19, which are reported as well expressed cell junction proteins conserved across different cell lines and/or tissue.

To detect RNA, cells in biological samples as discussed herein may be prepared for analysis of RNA and protein content using the methods and apparatus described herein. In certain aspects, cells are fixed and permeabilized prior to the hybridization step. Cells may be provided as fixed and/or permeabilized. Cells may be fixed by a crosslinking fixative, such as formaldehyde, glutaraldehyde. Alternatively or in addition, cells may be fixed using a precipitating fixative, such as ethanol, methanol or acetone. Cells may be permeabilized by a detergent, such as polyethylene glycol (e.g., Triton X-100), Polyoxyethylene (20) sorbitan monolaurate (Tween-20), Saponin (a group of amphipathic glycosides), or chemicals such as methanol or acetone. In certain cases, fixation and permeabilization may be performed with the same reagent or set of reagents. Fixation and permeabilization techniques are discussed by Jamur et al. in “Permeabilization of Cell Membranes” (Methods Mol. Biol., 2010).

A biological sample can include any sample of a biological nature that requires analysis. For example, samples may include biological molecules, tissue, fluid, and cells of an animal, plant, fungus, or bacteria. They may also include molecules of viral origin. Typical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Another typical source of biological samples are viruses and cell cultures of animal, plant, bacteria, fungi where gene expression states can be manipulated to explore the relationship among genes. In some cases, other samples can be interrogated, such as artificial samples. Certain aspects of the present disclosure are especially useful when interrogating samples of a human origin, and especially useful when interrogating samples of human peripheral blood.

A sample can be tagged or labelled with an element tag to facilitate determination of useful information upon interrogation through elemental analysis. An element tag is a detectable isotope (e.g., an element or isotope of an element) that can be detected through elemental analysis. In some cases, an element tag can include only the detectable isotope itself, although that need not always be the case. In some cases, an element tag can include a substrate to which one or more isotopes are coupled or in which one or more isotopes are otherwise contained. In this fashion, to create an element-tagged moiety, the detectable isotope can be coupled to the substrate separately from (e.g., before, simultaneously with, or after) the substrate being coupled to a moiety. For example, an element tag can include a polymer chain containing multiple pendant groups containing detectable isotopes (e.g., chelating groups containing metals), the polymer chain able to be bound to a moiety to element-tag that moiety. In another example, an element tag can include a bead or nanoparticle that can include one or more detectable isotopes therein or on the surface thereof, the bead or nanoparticle able to be bound to a moiety to element-tag that moiety. As used herein, an isotope or composition of isotopes located on a surface of a bead or nanoparticle includes isotopes or compositions of isotopes entrapped by a polymer on the surface, or bound to the surface (e.g., covalently or otherwise). In some cases, the bead or nanoparticle can include a solid metal core optionally encapsulated by a silicon oxide shell, or a polymeric core entrapping and/or chelating metal, and a polymer surface (e.g., using poly-L-lysine, PEG (polyethylene glycol), PEG MEA (methyl ether acrylate), PVMS (polyvinylmethyl siloxane), polydopamine, polystyrene and/or other suitable polymers). In certain aspects, the polymer (e.g., and/or its monomeric precursor) may be hydrophobic, for example may include acrylics, amides and imides, Carbonates, Dienes, Esters, Ethers, Fluorocarbons Olefins and/or aromatic groups, for example a phenolic group such as a catechol. The polymer may also provide a reactive group, such as an amine and/or thiol reactive group. For example, the reactive group may be a reactive functional group (e.g., a thiol, amine, thiol-reactive, amine-reactive, or click chemistry functional group) such as a quinone that forms upon polymerization. The polymer may form a thin film, such as a single layer film.

Beads of the subject application may be element encoded particles suitable for the attachment of biomolecules to enable massively multiplex bio-analytical methods. For example, polymeric beads may be made according to one or more aspects of US patent publication 20100144056, which is incorporated by reference and summarized below.

In one approach, one synthesizes polymer particles containing metal ions or atoms embedded in the interior. The polymer matrix of the particles may serve to encapsulate the metal ions, but at the same time provides colloidal stability in aqueous media. The polymer matrix of the particles minimizes the direct contact of the metal ions with the aqueous phase, and functional groups at the particle surface are available for attaching antibodies or other biomolecules to the particle. These functional groups can also be used to attach linker arms or spacer groups to which biomolecules can be attached. The polymer particles of interest have diameters ranging from about several nm to about 20 μm, with those of greatest interest having diameters of about 50 nm to about 500 nm.

Polymer particles may be synthesized containing metal ions or atoms embedded in the interior. The polymer matrix of the particles may serve to encapsulate the metal ions, but at the same time provides colloidal stability in aqueous media. In certain aspects, one can employ chelated lanthanide (or other metal) ions.

The polymer matrix of the particles minimizes the direct contact of the metal ions with the aqueous phase, and functional groups at the particle surface may be available for attaching antibodies or other biomolecules to the particle. These functional groups can also be used to attach linker arms or spacer groups to which biomolecules can be attached. Alternatively, a polymer film of a different structure may be synthesized on the surface of the bead, as discussed herein, and allow for attachment of the biomolecule.

In certain aspects, a bead is an assay barcoded bead as described herein. An interior of the bead may include an assay barcode, such as a distinguishable combination of metal isotopes. The interior of the bead may be any of a variety of suitable structures, such as a solid metal core, metal chelating polymer interior, nanocomposite interior, or hybrid interior. A solid metal core may be formed by subjecting a mixture (e.g., solution) of one or more metal elements and/or isotopes to high heat and/or pressure. A nanocomposite structure may comprise a combination (e.g., matrix) of nanoparticles/nanostructures (e.g., each comprising different physical properties and contributing one or more assay barcode elements/isotopes and/or providing scaffolding for other nanoparticles comprising assay barcode elements/isotopes). The interior of the bead may include a polymer entrapping the assay barcode metals and/or chelating assay barcode metals (e.g., through pendant groups such as DOTA, DTPA, or a derivative thereof). Suitable polymer backbones may be branched (e.g., hyperbranched) or form a matrix. In some aspects, the polymer may be formed in emulsion. The polymer may include subunits of styrene, acrylate, and any derivatives thereof or other polymers known in the art. In certain aspects, the interior of an assay bead may present an inert surface (e.g., such as a solid metal surface) that needs to be functionalized (e.g., by polymerization across the surface) prior to attachment to assay biomolecules (e.g., an oligonucleotide or antibody). Element tags have been provided for mass cytometry for conjugation to, or pre-conjugated to, biomolecules, for example an affinity reagent such as antibodies (e.g., antibodies or derivatives thereof such as antibody Fab fragment) that specifically binds a target analyte. Element tags for conjugation may include a polymer pre-loaded with a metal or alongside a separate metal solution for loading onto the polymer (e.g., prior to conjugation to a biomolecule). A polymer may include a backbone and a plurality of metal binding pendant groups, such as pendant groups comprising a chelator (e.g., DTPA, DOTA, or a derivative thereof). Element tags conjugated to a biomolecule such as an antibody may be pre-loaded with a metal. In certain aspects, the metal is an enriched isotope, such as an isotope of a lanthanide. Lanthanides are chemically similar, and lanthanide element tags (including those conjugated to antibodies) have been found to be stable in solution to an extent suitable for mass cytometry. However, non-lanthanide metals may not have similar stability. In certain aspects, the metal element or isotope thereof may be outside the lanthanide family, such as a non-lanthanide transition metal or a post-transition metal. Post-transition metals suitable for mass cytometry include elements with atomic numbers 48-50 (such as cadmium, indium, and/or tin) and 80-84. The stability of polymers loaded with a post-transition metal may be improved by lyophilization. Subject methods and kits include such non-lanthanide element tags lyophilized as described herein, provided separate from other element tags or in admixture with other element tagged biomolecules.
Medium weight elements (e.g., or their isotopes) may provide weaker signal by mass cytometry, such as when the element is near (e.g., within 30, 20 or 10 amu from) a high pass filter that removes Argon dimer from ICP and/or elements of organic molecules. Such medium weight elements or isotopes may have an atomic number between 39 and 52. Further, non-lanthanide elements such as certain transition metals and post-transition metals may not be chelated as readily to pendant groups on a polymer. As such, non-lanthanide metals (e.g., isotopes thereof) described herein may be useful for cell barcoding, for example, live cell barcoding with element tagged CD 45. For example, a live cell barcode may include lyophilized cadmium isotope tagged CD45.

The surface of an assay bead may comprise a polymer, linkers to space assay biomolecules away from the surface and/or add colloidal stability (e.g., PEG linkers), functional group(s) for attaching (or attached to) an assay biomolecule and/or sample barcode.

Element tags can be created with many different isotopes or unique combinations of isotopes. A unique element tag can have an element, isotope or combination of isotopes that is distinct (e.g., distinguishable by elemental analysis). For example, in a set of element tags, an element tag can be unique if it is distinguishable by mass (e.g., distinguishable by its composition of one or more isotopes) from the other element tags of that set. For clarity purposes, reference is made to an element tag or a unique element tag throughout, however it will be understood that certain aspects of the present disclosure make use of multiple copies of a unique element tag. Element tags can include one or more isotopes, such as one or more isotopes of a single element (e.g., ¹⁴²Nd and ¹⁴³Nd) or one or more isotopes across multiple elements (e.g., ¹⁴²Nd and ¹⁴¹Pr) In certain aspects, element tags may include metal atoms having an atomic mass above 80 amu. Such metal may be selected from noble metals, lanthanides, transition metals, and/or post-transition metals. Element tags may comprise a polymer. For example, a polymer may chelate metals (e.g., lanthanides) to metal binding pendant groups (such as DOTA or DTPA) on a polymer backbone, or may incorporate a metal (e.g., Tellurium) in a carbon backbone of the polymer itself, or may for around (entrap) a metal. Element tag may include a metal nanoparticle, such as a metal nanocrystal (e.g., functionalized on its surface to bind an affinity reagent). The metal of an element tag may be isotopically pure. The element tag may bind, or be functionalized to bind, a biomolecule (e.g., an affinity reagent such as an antibody).

Each element tag can include one or more discernable isotopes detectable through elemental analysis to determine presence of the element tag. Since different element tags can have unique isotopes or combinations of isotopes, elemental analysis can be used to identify the element tag based on its unique isotopic signature. The discernable isotopes can be selected to facilitate detection using elemental analysis. For example, discernable isotopes can be selected to be isotopes that are outside of the expected range of isotopes endogenous to a sample or isotopes introduced due to a particular elemental analysis technique. For example, biological samples would generally not inherently include metals that are greater than 80 amu, and thus element tags for use with such samples may include metals that are greater than 80 amu. In another example, certain types of inductively coupled plasma mass spectrometry (ICP-MS) make use of argon, and thus element tags for analysis by ICP-MS may make use of elements that have masses greater than argon, such as metals that are greater than 80 amu. Filtering techniques can be employed to exclude elements outside of the range of those selected as discernable elements in the element tags being used, thus automatically excluding elements that are endogenous to the sample and elements introduced during elemental analysis, if any. For example, in ICP-MS, a quadrupole filter can be applied to exclude ions having masses below 80 amu, thus keeping the detector from being overwhelmed by ions that are not of interest (e.g., ions that are not likely to be the detectable isotopes).

When a combination of isotopes for a given elemental tag are mentioned, that combination may be present on each instance of the element tag, or on different instances of the element tag that are in mixture together.

An element-tagged moiety is a chemical moiety that includes an element tag. Any suitable moiety can be used, although in certain aspects of the present disclosure, a moiety having a targeting function is used. A targeting function is an ability to bind to a target (e.g., a target protein or molecule). As used herein, referring to a moiety that binds a target is to be interpreted as a moiety that is able to bind to the target, whether or not the actual binding has already occurred. A targeting function can occur via specific binding (e.g., as in an antibody being specific for a particular antigen), a covalent bond, hybridization (e.g., of nucleic acids), or other suitable bonding mechanisms. A metal-containing moiety can be an element-tagged moiety that is tagged with a metal. Examples of suitable metal-containing moieties include metal-containing small molecules or drugs (e.g., cisplatin), histochemical stains (e.g., Ruthenium Red, Trichrome stain, or osmium tetroxide), metal-tagged oligonucleotides, and metal-tagged antibodies. In some cases, an element-tagged moiety can be a biomolecule, although that need not always be the case. A biomolecule can be any biological molecule, such as an affinity reagent (e.g., an antibody an aptamer), a nucleic acid (e.g., that hybridizes to a target), a lectin, a sugar, or other such molecule.

As used herein, the term affinity reagent can refer to a biological molecule (e.g., antibody, aptamer, lectin, or sequence-specific binding peptide) which is known to form highly specific non-covalent bonds with respective target molecules (e.g., peptides, antigens, or small molecules). Affinity reagent labeled with a unique element tag is an affinity reagent labeled with an element tag that is unique and distinguishable from a multitude of other element tags in the same sample. An affinity reagent can be considered an element-tagged moiety when conjugated to an element tag. While some embodiments and examples herein recite antibodies, an affinity reagent (e.g., antibody or non-antibody affinity reagent) may be used in place of an antibody in any such embodiment.

An element-tagged moiety can include or be bound to an element tag. In some cases, an element-tagged moiety can include an element tag, such as in the case of a cisplatin containing platinum as an element tag. In some cases, an element-tagged moiety can be bound to an element tag, such as an antibody bound to an element tag comprising a polymer scaffold with metal-chelating groups to which the distinguishable isotopes (e.g., metals) are bound. Use of a polymer scaffold can permit multiple isotopes to be bound to a single element-tagged moiety. Thus, a polymer scaffold can be used to provide a stronger elemental signal by including more copies of the distinguishable isotope. In some cases, a polymer scaffold can be used to facilitate providing a distinguishable combination of isotopes by having different isotopes contained at different places along the polymer scaffold. Such a polymer scaffold can contain a number of metal-chelating ligands attached to more than one subunit of the polymer. The number of metal-chelating groups capable of binding at least one metal atom in the polymer can be between approximately 1 and 10,000, such as 5-100, 10-250, 250-5,000, 500-2,500, or 500-1,000. At least one metal atom can be bound to at least one of the metal-chelating groups. The polymer can have a degree of polymerization of between approximately 1 and 10,000, such as 5-100, 10-250, 250-5,000, 500-2,500, or 500-1,000. Accordingly, a polymer based element tag can comprise between approximately 1 and 10,000, such as 5-100, 10-250, 250-5,000, 500-2,500, or 500-1,000 labelling atoms.

In some cases, an element tag can use a polymer selected from the group consisting of linear polymers, copolymers, branched polymers, graft copolymers, block polymers, star polymers, and hyperbranched polymers. The backbone of the polymer can be derived from substituted polyacrylamide, polymethacrylate, or polymethacrylamide and can be a substituted derivative of a homopolymer or copolymer of acrylamides, methacrylamides, acrylate esters, methacrylate esters, acrylic acid or methacrylic acid. The polymer can be synthesised from the group consisting of reversible addition fragmentation polymerization (RAFT), atom transfer radical polymerization (ATRP) and anionic polymerization. The step of providing the polymer can comprise synthesis of the polymer from compounds selected from the group consisting of N-alkyl acrylamides, N,N-dialkyl acrylamides, N-aryl acrylamides, N-alkyl methacrylamides, N,N-dialkyl methacrylamides, Naryl methacrylamides, methacrylate esters, acrylate esters and functional equivalents thereof. The polymer can be water soluble. This moiety is not limited by chemical content. However, it simplifies analysis if the skeleton has a relatively reproducible size (for example, length, number of tag atoms, reproducible dendrimer character, etc.). The requirements for stability, solubility, and non-toxicity are also taken into consideration. Thus, the preparation and characterization of a functional water soluble polymer by a synthetic strategy that places many functional groups along the backbone plus a different reactive group (the linking group), that can be used to attach the polymer to a molecule (for example, an affinity reagent), through a linker and optionally a spacer. The size of the polymer is controllable by controlling the polymerization reaction. Typically the size of the polymer will be chosen so as the radiation of gyration of the polymer is as small as possible, such as between 2 and 11 nanometers. The length of moieties bound to the element tag can be on the order of approximately 10 nanometers, and therefore an excessively large polymer tag in relation to the size of the moiety to which the element tag is bound may sterically interfere with the targeting function of that moiety.

In some cases, the metal-chelating group that is capable of binding at least one metal atom can comprise at least four acetic acid groups. For instance, the metal-chelating group can be a diethylenetriaminepentaacetate (DTPA) group; a 1,4,7,10-tetraazacyclododecane-1,4,7,10-tetraacetic acid (DOTA) group; or a DTPA or DOTA derivative group. Alternative groups include Ethylenediaminetetraacetic acid (EDTA) and ethylene glycol-bis(β-aminoethyl ether)-N,N,N′,N′-tetraacetic acid (EGTA). The metal-chelating group can be attached to the polymer scaffold through an ester or through an amide. Examples of suitable metal-chelating polymers include the X8 and DM3 polymers available from Fluidigm Canada, Inc.

An element-tagged moiety can be labelled with an elemental tag that is discernable through elemental analysis. Element-tagged moieties can be selected to bind to or couple to any suitable structure (e.g., target) desired to be labelled by the element label and then detected through elemental analysis. For example, suitable element-tagged moieties can include oligonucleotide probes for hybridization, cell state probes (e.g., cisplatin as a viability marker or organotellurium as a hypoxia or synthesis marker), or other suitable molecules.

In certain aspects of the present disclosures, an element-tagged antibody is used as an element-tagged moiety. Antibodies can be specific to specific cell types. A cell type (e.g., cell population) can refer to a population of cells or a certain subset of a cell population. An antibody that is specific to a cell type can have an antigen binding site (e.g., a paratope) that is capable of binding to a molecule or antigen (e.g., an epitope of an antigen) present in or on that particular cell type. In some cases, antibodies can be specific to targets on or within peripheral blood, such as surface targets or intracellular targets of peripheral blood mononuclear cells (PBMCs).

Certain aspects of the present disclosure describe components as being in mixtures or admixtures. As used herein, the term mixture or admixture includes components that are combined together (e.g., physically located together in the same container or enclosure). A container or enclosure includes any suitable volume of space that is separable from another volume of space. A container or enclosure can be sealed, such as hermetically sealed. Examples of suitable containers or enclosures include tubes (e.g., test tubes), boxes, pouches, wells of a multi-well plate, or other such containers.

In some cases, various aspects of the present disclosure can make use of calibration materials to facilitate calibrating the elemental analyzer. Calibration material can be any suitable metal or metal-containing materials usable to provide calibration to the elemental analyzer. In some cases, calibration materials can be metal-containing beads, with a mixture of different isotopes from different elements over a range of elemental masses. Calibration materials can contain known quantities of one or more known isotopes.

Certain aspects of the present disclosure make use of element-tagged antibodies to enable highly multiplexed, single-cell analysis of biological samples using elemental analysis, such as mass cytometry. Such techniques can allow for numerous markers in a single panel to be resolved without the need for compensation. In some cases, more than 30 markers can be resolved in a single panel.

The large number of detection channels (e.g., more than 20 but less than 60) enabled by mass cytometry provides an opportunity to leverage combinatorial assays. For example, the number of targets detected, which is normally equal to the number of channels, can be increased by relating antibody panels to one another by shared subsets of biomolecules (e.g., antibodies) and using the same mass tag for antibodies that differ between panels. For example, the number of targets detected in a given sample of cells (e.g., dataset) may at least 1.2, 1.5 or 2 times as many as detection channels used.

Cells may be stained with a shared panel, such that cell populations identified in the distinct panels can be related to one another, provided that mass tag channels are left over for use in distinct panels for individual aliquot. As panels use some mass tags for different antibodies (to detect different targets), a sample barcode may be applied to cells in an aliquot as a “panel barcode” that identifies which mass tag relates to which antibody. Alternatively, a “master” shared panel may stains a partition (aliquot) of cells of a sample and be used (and be necessary) to relate populations identified by other panels to one another. The master shared panel may have different or only partially overlapping subsets of antibodies that are shared with each of a plurality of distinct panes that each stain a different aliquot of cells. There may be 2 or more, 3 or more, 4 or more, 5 or more, or 10 or more distinct panels that share antibodies with the shared panel. A distinct panel may have more mass tags in common with another distinct panel than antibodies in common.

The workflow would involve splitting a single sample (e.g., human PBMCs) into separate aliquots, and staining each aliquot with a different cell-type specific panel. The same subset of mass channels would be used for different cell type specific markers depending on the panel (for example, a T cell panel may not have any or some of the B cell markers in the B cell panel, and vice versa). An Amazon-type software solution would identify cells that were stained by different panels, and could combine them into the same data set.

Barcoding of cells by panel may allow software to automatically identify what panel each cell was stained with. Aspects of the subject application include such software, including software that also performs other automated analysis described in this application.

A shared subset of markers that identify all the basic cell types could be its own panel, or a subset to all panels. This could be used to identify the relative abundance of each of the major cell types, and then be used in combination with cell type specific panels to identify the relative abundance of specific cell types (populations) across panels.

The software solution may throw out cells events that are not of the cell type focused on in the panel, so that each cell event was stained for the targets of interest. For example, any B cell events in the T cell panel may be removed. This could be done after calculating relative abundances of the major cell types, as described in the above bullet point. One or more distinct panels could have investigative markers. Some or all of these may be shared across panels. There may be one or more unused channels for a user to detect their own targets of interest.

In certain aspects, a method of elemental analysis includes: separating cells of a sample into a plurality of partitions; staining cells with a shared panel of mass tagged antibodies; staining cells of separate partitions with distinct panels of mass tagged biomolecules, wherein individual distinct panels comprise biomolecules that are not in other distinct panels but that are tagged with a mass tags present in the other distinct panels; and/or interrogating the sample using elemental analysis to detect a presence of the distinct mass tags on individual cells. The shared panel may be conserved across two or more partitions, such as three or more, four or more, or five or more partitions. The shared panel may detect positively expressed surface targets that distinguish parent populations, wherein the parent populations together cover the majority of immune cells. One or more (e.g., two or more, three or more, or four or more) individual distinct panels each detect positive markers that distinguish sub-populations within one of the parent populations, but do not distinguish sub-populations for the majority of immune cells.

The shared panel may not stain all partitions (e.g., may only stain one), and the shared panel comprises different subsets of antibodies that are identical to antibodies of two or more distinct panels.

Individual distinct panels may subpopulations within a parent population identified by the shared panel, wherein a parent population is selected from a population including or consisting of T cells, CD4 T cells, regulatory T cells, CD8 T cells, NK cells, B cells, dendritic cells, and/or monocytes/macrophages. Alternatively or in addition, distinct panels may identify cell function for example when the distinct panel detects targets involved in intracellular signaling, cytokine production, and/or cell cycle.

In certain aspects, oligonucleotides are used in addition or instead of antibodies in a distinct panel, for example when the oligonucleotides specifically hybridize, directly or indirectly, to target RNA. For example, oligonucleotides may hybridize to RNAs encoding cytokines as described herein.

Wherein staining with distinct panels comprises signal amplification by staining separate partitions with oligonucleotide tagged antibodies and hybridizing, directly or indirectly, mass tagged oligonucleotides to the oligonucleotides tagging the antibodies.

One or more of the shared panel and the distinct panels may be lyophilized, as described for panels herein.

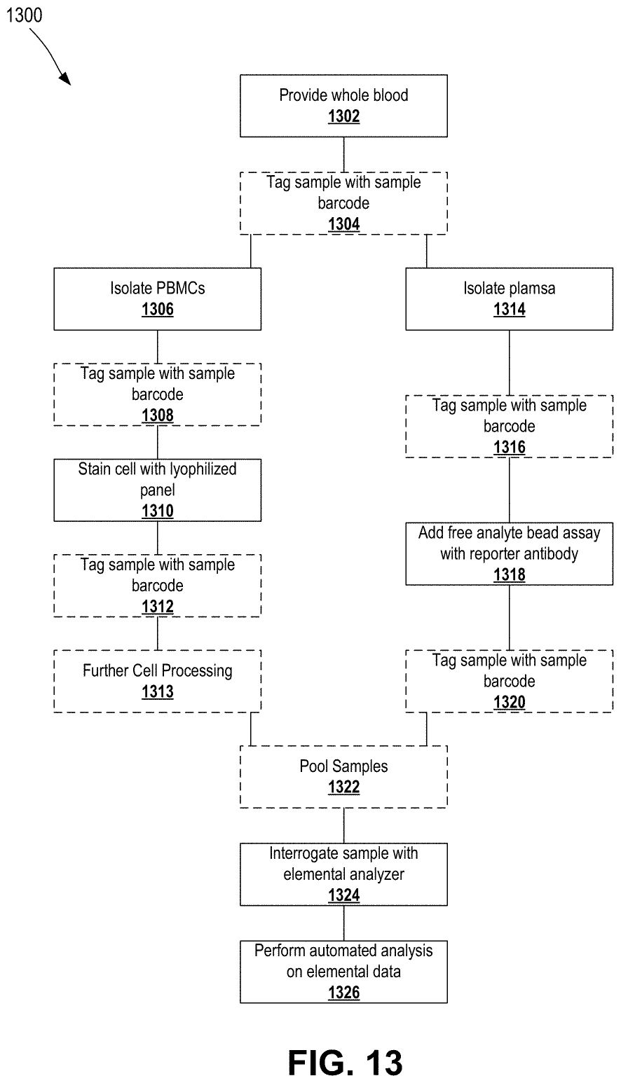

Cells are stained with the shared panel prior to separation into partitions, or the cells are partitioned prior to staining with the shared panel (e.g., wherein the shared panel is in admixture with each distinct panel). A method may further include labeling partitioned cells with a panel barcode that identifies the distinct panel (and therefore target associated with mass tags of the panel), e.g., wherein the panel barcode is provided in admixture with its distinct panel prior to addition to the partitioned cells. Partitions are combined after labeling with the panel barcode and before interrogation. Panel barcodes could be applied in a similar way to sample barcodes illustrated in FIG. 13, or may be administered in addition to sample barcodes.

The above methods may be performed on one or more additional samples (which are themselves split into aliquots for separate staining), labeling the samples with distinct sample barcodes, and combining the barcoded samples prior to interrogation. The barcoded samples can be combined prior to separation into partitions.

Methods may further include classifying individual interrogated cell into cell populations based on the shared panel and its distinct panel. For example, the shared panel may identify the T-cell parent population, but a distinct T-cell panel used for one of the partitions may identify sub-populations within that parent population. Other populations and panels are discussed in this application a suitable for these methods.

Classification may be by automated software trained on the shared panel and distinct panels (e.g., on similar samples, such as a sample including PBMCs). Classification may be by gating, by a trained clustering algorithm operating in highly dimensional space (where dimensions are related to the number of surface markers used for classification), or by a neural network.

A distinct panel may be used to identify sub populations of a parent population identified by a shared panel. A method may further include integrating interrogated cell populations identified based on different distinct panels into the same data set based on the shared panel. Integrating may include: discarding data of cells identified to be of a parent population that the shared panel does not identify sub-populations for; identifying the proportion of cells in the original sample that are in sub-populations identified by distinct panels of separate partitions; and/or representing expression of targets detected by different distinct panels for the same parent population detected by the shared panel.

Methods may further include stimulating cells of different samples or sample partitions under different conditions prior to staining.

A kit may comprise a shared panel and distinct panel packaged for any of the above methods. A shared panel and distinct panels may be packaged in a kit as described in the above methods.

In certain aspects, a kit for elemental analysis (e.g., mass spectrometric analysis) may include a shared panel comprising a plurality of conjugated antibodies. The shared panel may include a plurality of antibodies conjugated to a distinct mass tag, wherein each distinct mass tag is distinguishable based on its isotopic composition. The plurality antibodies may in mixture. The plurality of distinct panels may include mass tagged biomolecules (e.g., antibodies and/or oligonucleotides), wherein individual distinct panels include biomolecules not present in one or more other panels but that is tagged with a mass tag present in the one or more other panels.

Certain aspects of the present disclosure enable lyophilization of an antibody panel to achieve a panel having high stability. Stability of a lyophilized antibody panel can be described in terms of a comparison of an ion count of element tag isotopes of a sample assayed using the lyophilized antibody panel versus the same sample assayed using a non-lyophilized antibody panel. The higher stability of a lyophilized antibody panel over a non-lyophilized antibody panel can be seen as a higher ion count of the element tags from the lyophilized antibody panel than the ion count of element tags from the non-lyophilized antibody panel. Further, stability can be described as a function of damage to the antibody (e.g., damage affecting the binding activity of the antibody) and/or a function of metal retention of the metal atoms of the distinguishable isotopes on the element tag and/or element-tagged moiety. For example, a stable panel would exhibit little to no damage affecting the binding activity of the antibody and/or little to no loss of metal atoms from the element tag and/or element-tagged moiety. The lyophilized panels described herein exhibit better stability than a non-lyophilized panel of the same makeup.

Element tags have been provided for mass cytometry for conjugation to, or pre-conjugated to, antibody biomolecules. Element tags for conjugation may include a polymer pre-loaded with a metal or alongside a separate metal solution for loading onto the polymer (e.g., prior to conjugation to a biomolecule). A polymer may include a backbone and a plurality of metal binding pendant groups, such as pendant groups comprising a chelator (e.g., DTPA, DOTA, or a derivative thereof). Element tags conjugated to a biomolecule such as an antibody may be pre-loaded with a metal. In certain aspects, the metal is an enriched isotope, such as an isotope of a lanthanide. Lanthanides are chemically similar, and lanthanide element tags (including those conjugated to antibodies) have been found to be stable in solution to an extent suitable for mass cytometry. However, non-lanthanide metals may not have similar stability. In certain aspects, the metal element or isotope thereof may be outside the lanthanide family, such as a non-lanthanide transition metal or a post-transition metal. Post-transition metals suitable for mass cytometry include elements with atomic numbers 48-50 (such as cadmium, indium, and/or tin) and 80-84. The stability of polymers loaded with a post-transition metal may be improved by lyophilization. Subject methods and kits include such non-lanthanide element tags lyophilized as described herein, provided separate from other element tags or in admixture with other element tagged biomolecules.

Medium weight elements (e.g., or their isotopes) may provide weaker signal by mass cytometry, such as when the element is near (e.g., within 30, 20 or 10 amu from) a high pass filter that removes Argon dimer from ICP and/or elements of organic molecules. Such medium weight elements or isotopes may have an atomic number between 39 and 52. Further, non-lanthanide elements such as certain transition metals and post-transition metals may not be chelated as readily to pendant groups on a polymer. As such, non-lanthanide metals (e.g., isotopes thereof) described herein may be useful for cell barcoding, for example, live cell barcoding with element tagged CD 45. For example, a live cell barcode may include lyophilized cadmium isotope tagged CD45.

Generally, generating and acquiring large multiplexed panels can involve optimization of antibody titers, full panel verification, sample preparation, and analysis. Preparation of a staining cocktail from numerous individual tubes (e.g., 30 different tubes) is tedious and may be prone to errors. Additionally, pre-prepared staining cocktails, especially when based on metal-tagged antibodies, may be prone to damage over time and may exhibit poor stability. For example, anon-lyophilized metal-tagged antibody panel containing multiple antibodies can remain stable for about a week or less, whereas certain aspects of the present disclosure relate to similar lyophilized antibody panels that can remain stable for approximately a year or more. Thus, a lyophilized antibody panel can facilitate creation and distribution of antibody panels on a larger scale, as well as permit long-term storage of an antibody panel, improving consistency in subsequent assays making use of the same panel.

Sample preparation according to the subject methods may be performed at least in part by an automated sample preparation system. Such a system may process the sample prior to staining, may contact the sample with one or more antibody panels, assay beads, and/or barcodes, may perform centrifugation and wash steps, and/or may deliver the sample to a mass cytometry system.

Therefore, certain aspects of the present disclosure relate to a lyophilized multiplex (e.g., 30-plex) immunophenotyping panel contained in a single enclosure (e.g., a single tube), which can be used with an efficient workflow and an automated software solution for human whole blood analysis using mass cytometry. In some cases, the panel can focus on T cell lineage while also capturing other relevant immune populations. Blood can be added directly to the lyophilized antibody tube, followed by red blood cell (RBC) lysis, wash, and fixation steps, and finally data acquisition of stained samples on an elemental analyzer (e.g., an ICP-MS system).

A software tool can accept the elemental analyzer data and automatically generate reports on various cytometric information, such as the number of live cells, the percentage of specific cell populations, staining intensities, histograms, 2D dot plots, and t-distributed stochastic neighbor embedding (tSNE) graphs. The software may automatically calculate frequencies of populations that are comparable to manual gating. The automated software can eliminate user bias that may occur during manual analysis (e.g., manual gating), as well as significantly reduce the amount of time required to analyzer each data file. The panel and software tool can enable researchers to streamline immunophenotyping of whole blood while accurately and reproducibly monitoring changes in immune cell subsets in patient samples. In some cases, the software tool can be automatically loaded with the appropriate element tags to enable rapid decoding of element analyzer data into useful results.

Antibodies and other element-tagged moieties can be used to identify the presence of cell targets in a sample. The antibody or element-tagged moiety can have a targeting function for the cell target, and thus remain with the sample after the sample has been washed. Thus, any detection of the distinct isotope or combination of isotopes associated with a particular element tag on an antibody or element-tagged moiety is indicative of the presence of that cell target. Cell targets can be surface targets or intracellular targets. Examples of intracellular targets can include cell cycle and proliferation targets, intracellular signaling targets, and intracellular cytokine targets. Since permeabilizing a cell can sometimes disrupt cell surface markers, staining of a sample with antibodies or element-tagged moieties associated with surface targets can be performed before permeabilizing. However, since permeabilizing a cell is sometimes necessary for proper access to intracellular targets, staining of a sample with antibodies or element-tagged moieties associated with intracellular targets can be performed after permeabilizing.

In some cases, an intercalator can be used. In some cases, the intercalator can be an element-tagged moiety. The intercalator can comprise or be coupled to an element of an element tag. For example, iridium can be used as an intercalator and can simultaneously function as its own element tag. Thus, detection of iridium during interrogation is indicative of presence of the intercalator. Other suitable intercalators include rhodium and cisplatin. Intercalators can be included in a lyophilized panel or provided separate from the lyophilized panel. If an intercalator is mixed with sample cells prior to permeabilization, the intercalator can be a cell viability stain, indicating whether or not the cell is a live cell. In such cases, detected presence of the intercalator during interrogation is indicative of a dead cell. However, if an intercalator is mixed with sample cells after permeabilization, the intercalator can act as a cell identification stain or cell presence stain, which can indicate the presence of a cell, since the intercalator should enter all permeabilized cells. In such cases, detected presence of the intercalator during interrogation is indicative that a cell is being analyzed by the elemental analyzer. In some cases, an intercalator, such as rhodium, may be included alongside, or in mixture with, a lyophilized antibody panel.

The antibodies selected for use in a lyophilized panel can be selected to perform certain assays. In some cases, lyophilized panels or lyophilized panel subsets can be created for various assays, thus facilitating performing of the desired assay(s) by simply selecting the appropriate panel or combination of panel subsets. Various panels or panel subsets are described herein for use with certain aspects of the present disclosure. Each panel or panel subset described with reference to a list of possible antibodies can include two or more antibodies from that list or any number of antibodies from that list, up to and including all antibodies from that list. As used herein, a panel subset can include any combination of antibodies from a panel described herein. In some cases, a panel subset can be combined with different panels or panel subsets to create one or more of the panels described herein.

In some cases, the lyophilized panel can include two or more antibodies from the list including Cd45, CD45RA, CD45RO, Cd123, CD4, CD8a, CD11C, CD57, CXCR3, CD185, CD38, CD56, CD3, CD20, CD66b, HLA-DR, IgD, CD27, CD28, CD127, CD19, CD16, CD161, CD194, CD25, CD294, CD197, CD14, CCR6, and TCR δγ. In some cases, the lyophilized panel can include at least 3, 4, 5, 6, 7, 8, 9, 10, 11, 12, 13, 14, 15, 16, 17, 18, 19, 20, 21, 22, 23, 24, 25, 26, 27, 28, 29, or 30 antibodies from this list. As disclosed herein, certain aspects of the present disclosure are useful for incorporating various different element-tagged antibodies into a single lyophilized panel. In some cases, this panel can be especially useful for immunophenotyping human peripheral blood. In some cases, this panel may be a shared panel in a kit or method comprising distinct panels for labeling partitioned (e.g., aliquots) of cells from the same sample, as described further herein. Such distinct panels may include one or more of the panels below.

In some cases, the lyophilized panel can include a leukemia panel and/or a lymphoma panel comprising antibodies especially suitable for determining cytometric information regarding leukemia cells and/or lymphoma cells.

Various example lyophilized panels are described herein. For descriptive purposes, panels can be described in terms of antibodies and targets, with targets denoted in parentheticals. In some cases, different antibodies can be substituted for any given antibody as long as the different antibody is specific to the target of the substituted antibody. In certain embodiments, multiple lyophilized panels may be provided (e.g., in a kit), such that they can be combined depending on the sample and/or application.

In some cases, a lyophilized panel for human acute myeloid leukemia (AML) phenotyping can include antibodies (and targets) from the list including HIB19 (CD19), 104D2 (CD117), ICRF44 (CD11b), 10.1 (CD64), CD7-6B7 (CD7), 6H6 (CD123), HI30 (CD45), WM53 (CD33), Clone (Target), W6D3 (CD15), 581 (CD34), UCHT1 (CD3), IM7 (CD44), HIT2 (CD38), L243 (HLA-DR), and 12G5 (CXCR4). In some cases, this panel can be especially useful for phenotyping AML. AML, the most common type of acute leukemia in adults, is a malignancy arising within the bone marrow due to a disruption of normal hematopoiesis. AML arises within precursors of myeloid, erythroid, megakaryocytic and monocytic cell lineages due to the acquisition of chromosomal rearrangements and multiple gene mutations. The immunophenotype of AML is highly heterogeneous; markers frequently expressed by AML include CD15, CD33, CD34 and CD64.

In some cases, a lyophilized panel for human B cell phenotyping can include antibodies (and targets) from the list including HIB19 (CD19), 1A6-2 (IgD), 2H7 (CD20), polyclonal (IgA), BL13 (CD21), L128 (CD27), HIB22 (CD22), CB3-1 (CD79b), HIT2 (CD38), ML5 (CD24), MHM-88 (IgM), and L243 (HLA-DR). In some cases, this panel can facilitate the identification and phenotyping of human B cells, including naïve, memory, transitional, and plasma B cell populations.

In some cases, a lyophilized panel for identifying cell cycle information can include antibodies (and targets) from the list including N/A (S-Phase (IdU)), J112-906 (pRb [Ser807/811]), GNS-1 (CyclinB1), B56 (Ki67), and HTA28 (pHistone H3 [Ser28]). In some cases, this panel can facilitate assessment of cell cycle status: proliferation, G0 (senescent), G1, S-Phase, G2, and M-phase (mitosis). This panel can be especially useful in combination with other panels to identify cell cycle status as well as other cytometric information.

In some cases, a lyophilized panel for human helper T cell phenotyping can include antibodies (and targets) from the list including G034E3 (CCR6), NP-6G4 (CCR5), RPA-T8 (CD8), C398.4A (ICOS), HI100 (CD45RA), UCHT1 (CD38), G025H7 (CXCR3), 205410 (CCR4), GP-3G10 (CD161), UCHL1 (CD45RO), 2A3 (CD25), RF8B2 (CXCR5), SK3 (CD4), EH12.2H7 (PD-1), and A019D5 (CD127). In some cases, this panel can be used for the identification and phenotyping of human CD4+ helper T cell subsets, including T helper 1 (T_{H}1), T_{H}2, T_{H}17, T_{H}22, T follicular helper (TFH), and T regulatory (T_{REG}). Differentiation of CD4+ T cells into functionally distinct helper T subsets can be important for normal immunoregulation. These subsets are specified by extrinsic and intrinsic cues, and the resultant cell populations acquire stable phenotypes defined by the expression of signature cytokines, ‘master regulator’ transcription factors, and characteristic cell surface phenotypes.

In some cases, a lyophilized panel for basic human peripheral blood phenotyping can include antibodies (and targets) from the list including 2H7 (CD20), HI30 (CD45), M5E2 (CD14), 3G8 (CD16), SK1 (CD8), UCHT1 (CD3), and SK3 (CD4). This panel can be useful for assaying fresh or frozen human whole blood or PBMCs. This panel can be used to identify CD4+T, CD8+T, B cells, Monocytes, NKs, and Granulocytes.

In some cases, an additional lyophilized panel for basic human peripheral blood phenotyping can include antibodies (and targets) from the list including RPA-T4 (CD4), RPA-T8 (CD8a), 2H7 (CD20), 3G8 (CD16), HI30 (CD45), M5E2 (CD14), and UCHT1 (CD3). This panel can be useful for assaying fresh or frozen human whole blood or PBMCs. This panel can be used to identify CD4+T, CD8+T, B cells, Monocytes, NKs, and Granulocytes.

In some cases, a lyophilized panel for human peripheral blood phenotyping can include antibodies (and targets) from the list including UCHT1 (CD3), RPA-T4 (CD4), RPA-T8 (CD8a), Bu15 (CD11c), M5E2 (CD14), 3G8 (CD16), HIB19 (CD19), 2H7 (CD20), 0323 (CD27), HIT2 (CD38), HI30 (CD45), HI100 (CD45RA), VI-PL2 (CD61), CD66a-B1.1 (CD66), 6H6 (CD123), HIR2 (CD235a/b), and L243 (HLA-DR). This panel can be useful for assaying fresh or frozen human whole blood or PBMCs. This panel can be useful to identify major peripheral blood cellular subsets including Granulocytes, Basophils, Plasmacytoid Dendritic Cells, Natural Killer Cells, Effector T Killer Cells, Naïve T Killer Cells, Activated T Killer Cells, Memory T Killer Cells, Effector T Helper Cells, Naïve T Helper Cells, Activated T Helper Cells, Memory T Helper Cells, Memory B Cells, Naïve B Cells, Plasma B Cells, Myeloid Dendritic Cells, Non-Canonical Monocytes Canonical Monocytes and Platelets.

In some cases, a lyophilized panel for human T cell phenotyping can include antibodies (and targets) from the list including HI111 (CD11a), RPA-T4 (CD4), RPA-T8 (CD8a), 3G8 (CD16), 2A3 (CD25), HI30 (CD45), G043H7 (CCR7), FN50 (CD69), UCHL1 (CD45RO), BJ18 (CD44), 0323 (CD27), HI100 (CD45RA), UCHT1 (CD3), HCD57 (CD57), L243 (HLA-DR), and A019D5 (CD127). This panel can be useful for assaying fresh or frozen human whole blood or PBMCs. This panel can be useful for the identification of major T Cell subsets including naïve, central memory, effector, and effector memory CD4+ and CD8+ cells, and also classify the activation and homing status of these subtypes.

In some cases, a lyophilized panel for expanded human T cell phenotyping can include antibodies (and targets) from the list including TS1/8 (CD2), UCHT2 (CD5), CD7-6B7 (CD7), SN4 C3-3A2 (CD9), CD28.2 (CD28), 9F10 (CD49d), HP-3G10 (CD161), 205410 (CCR4), NP-6G4 (CCR5), and G025H7 (CXCR3). This panel can be useful for assaying fresh or frozen human whole blood or PBMCs. This panel can be especially useful when combined with a human T cell phenotyping panel, in which case the combined panels can be used to identify all major T cell subsets including naïve, central memory, effector, and effector memory CD4+ and CD8+ cells, naïve and memory Tregs, TH1 and TH2 cells, and to classify the activation and homing status of these subtypes.

In some cases, a lyophilized panel for human embryonic stem (ES) cell or induced pluripotent stem (iPS) cell phenotyping can include antibodies (and targets) from the list including TRA-1-60 (TRA-1-60), 030-678 (Sox2), 40/Oct-3 (Oct-3/4), N31-355 (Nanog), IM7 (CD44), and 9E10 (c-Myc).

In some cases, a lyophilized panel for human hematopoietic stem and progenitor cell phenotyping can include antibodies (and targets) from the list including HI10a (CD10), WM15 (CD13), 581 (CD34), G0H3 (CD49f), 104D2 (CD117), DL-101 (CD138), and 12G5 (CXCR4). This panel can be useful for the identification and phenotyping of hematopoietic progenitor populations, including hematopoietic stem cells (HSC), within human bone marrow and cord blood. This panel can be usefully combined with a human peripheral blood phenotyping panel so that lineage-positive cells can be excluded from gating strategies.

In some cases, a lyophilized panel for human intracellular cytokine I assays can include antibodies (and targets) from the list including B27 (IFNγ), MQ1-17H12 (IL-2), MP4-25D2 (IL-4), TRFK5 (IL-5), MQ2-13A5 (IL-6), N49-653 (IL-17A), SHLR17 (IL-17F), B (Granzyme), B-D48 (Perforin), D21-1351 (MIP1β), and Mab11 (TNFα). This panel can be useful for assaying fresh or frozen human whole blood, PBMCs, or cell lines. This panel can facilitate measurement of 11 major cytokines as well as cytolytic proteins, Granzyme B, and Perforin. This panel can be combined with a human peripheral blood phenotyping panel to allow for the comprehensive immunophenotyping of cytokine-expressing cells.

In some cases, a lyophilized panel for human regulatory T cell phenotyping can include antibodies (and targets) from the list including 9F10 (CD49D), RPA-T4 (CD4), 205410 (CCR4), HI100 (CD45RA), UCHT1 (CD3), A1 (CD39), PCH101 (Foxp3), DX2 (CD95), UCHL1 (CD45RO), 2A3 (CD25), 14D3 (CD152), L243 (HLA-DR), and A019D5 (CD127). This panel can be useful for identifying regulatory T cells. Regulatory T cells (Tregs) are a suppressive subset of CD4+T helper (Th) cells important for the regulation of immune responses. Tregs are defined by expression of the transcription factor Foxp3. Additional Treg markers include constitutive expression of the high-affinity IL-2Rα chain (CD25) and cytotoxic T lymphocyte-associated antigen 4 (CTLA-4), along with low expression of the IL-7Rα chain (CD127). CD4+CD25+Foxp3+ Tregs can be divided into two main types: thymically derived Tregs (tTregs) and peripherally derived Tregs (pTregs).

In some cases, a lyophilized panel for human monocyte/macrophage phenotyping can include antibodies (and targets) from the list including HIB19 (CD19), ICRF44 (CD11b), CD7-6B7 (CD7), CD66a-B1.1 (CD66), 5-271 (CD36), GHI/61 (CD163), HI30 (CD45), Bu15 (CD11c), M5E2 (CD14), 3G8 (CD16), HIT2 (CD38), 15-2 (CD206), WM53 (CD33), UCHT1 (CD3), and L243 (HLA-DR). This panel can be used to identify and phenotype monocytes and macrophages. Monocytes circulate in the blood, bone marrow, and spleen and constitute approximately 2-12% of total human leukocytes. Monocytes have been considered as the systemic reservoir of myeloid precursors for renewal of tissue macrophages and dendritic cells, although there are DC and macrophage subpopulations that originate independently of monocytes. Recruited monocytes are innate effectors of the immune response to microbes, and they kill pathogens via phagocytosis, production of reactive oxygen species (ROS), nitric oxide (NO), myeloperoxidases and inflammatory cytokines. Monocytes can be categorized based on the expression of CD14 and CD16 as “classical” (CD14+CD16-), intermediate (CD14+CD16+) and non-classical (CD14loCD16+).

In some cases, a lyophilized panel for signaling assays can include antibodies (and targets) from the list including 47 (pSTAT5), 58D6 (pSTAT1), D3F9 (p38), 4/P-Stat3 (pSTAT3), L35A5 (Iκβα), D13.14.4E (pERK1/2), and N7-548 (pS6). This panel can be useful for quantifying basal and induced phosphorylation of multiple key signaling pathways: JAK/STAT, NFκB, and MAPK. This panel can be combined with other panels to measure cell signaling in heterogeneous samples, such as blood or splenocytes. Alternatively, it may be used as a standalone panel when measuring homogeneous samples such as cell lines.

In some cases, a lyophilized panel for basic mouse spleen/lymph node phenotyping can include antibodies (and targets) from the list including 30-F11 (CD45), M1/70 (CD11b(MAC1)), 145-2C11 (CD3e), 53-6.7 (CD8a), RM4-5 (CD4), and RA3-6B2 (B220). This panel can be useful to identify CD4+T, CD8+T, B cells, macrophages, and monocytes in fresh or frozen isolated mouse splenocytes and thymoctyes.

In some cases, a lyophilized panel for mouse spleen/lymph node phenotyping can include antibodies (and targets) from the list including RB6-8C5 (Ly6G/C (GM), N418 (CD11c), H1.2F3 (CD69), 30-F11 (CD45), M1/70 (CD11b (MAC1)), 6D5 (CD19), 3C7 (CD25), 145-2C11 (CD3e), TER119 (TER-119), MEL-14 (CD62L), 53-6.7 (CD8a), H57-597 (TCRβ), PK136 (NK1.1), IM7 (CD44), RM4-5 (CD4), and RA3-6B2 (B220). This panel can be useful to identify major mouse spleen/lymph cellular subsets including effector CD4+T, effector memory CD4+T, central memory CD4+T, activated CD4+T, effector CD8+T, effector memory CD8+T, central memory CD8+T, Tregs, plasmacytoid DC, myeloid DC, erythrocytes, macrophages, monocytes, NK cells, and granulocytes in fresh or frozen isolated mouse splenocytes and thymoctyes.

In some cases, a lyophilized panel for mouse intracellular cytokine I assays can include antibodies (and targets) from the list including XMG1.2 (IFNy), JES6-5H4 (IL-2), 11B11 (IL-4), TRFK5 (IL-5), MP5-20F3 (IL-6), JESS-16E3 (IL-10), TC11-18H10.1 (IL-17A), and MP6-XT22 (TNFa). This panel can be useful for assaying major mouse cytokines in fresh or frozen sources of mouse leukocytes, including splenocytes, thymocytes, bone marrow and lymph node cells, or cell lines. This panel can be used with a mouse spleen/lymph node phenotyping panel to allow for the comprehensive immunophenotyping of cytokine-expressing cells.

In some cases, additional panels or panel subsets can include antibodies specific to regulator T cell surface markers (e.g., PD-1, CTLA-4, GITR, CXCR3, IL-12R, IL-4R, CRTH2, IL-17Rb, IL-23R, CCR6, IL-1Rb, OX40L, CD40L, SLAM, IL-21R, ICOS, CXCR5, TIM3, 1B11, LAG3, and/or BTLA); intracellular regulatory T cell transcription factor markers (e.g., FoxP3, RORgT, T-bet, Bcl6, and/or GATA3); intracellular cytokine and chemokine markers (e.g., IFNg, IL-2, IL-4, IL-6, IL-7, IL-12, IL-15, IL-17A, IL-22, IL-23, TGFb, TNFa, Perforin, and/or Granzyme b); drugable target markers (e.g., BTLA, GITR, 4-1BB, OX40, TIGIT, Helios, and/or ICOS); and/or myeloid derived suppressor cell markers (e.g., CD11b, CD15, and/or CD33).

A CD 4 T cell panel may include at least two of CCR6 (G034E3), CD45RA (HI100), CD4 (RPA-T4), LAG3 (polyclonal; R&D Systems), CCR4 (205410), CD62L (DREG-56; BioLegend), CD49b (AK-7; BD Biosciences), CXCR3 (G025H7), CD161 (HP-3G10), TIGIT (MBSA43; eBioscience), ICOS (DX29; BD Biosciences), CD226 (11A8; BioLegend), CD8α (SK1), CD25 (2A3), CTLA-4 (14D3), CXCR5 (51505), CD3 (UCHT1; BioLegend), PD-1 (EH12.2H7), optionally in the form of the clone listed in parentheses. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

An NK panel may include at least two of CD27, CD4, CD8, CD57, TRAIL, KIRDL2/L3/S2, CD16, CD117, KIR2Ds4, LILRB1, NKp46, NKG2D, NKG2C, 2B4, NKp30, CD122, KIR3DL1, CD94, CCR7, KIRDL3, NKG2A, HLA-DR, KIR2DL4, CD56, CD45, KIR2DL5, and CD25. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

A CD8 T-cell panel may include at least two of CD3, CCR7, CD11a, CD7, CD8, CD27, CD28, CD29, CD43, CD45RA, CD45RO, CD49d, CD57, CD62L, KLRG1, and HLA-DR. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

A Treg panel may include at least two of CCR6 G034E3, LAP TW4-2F8, CD45RA HI100, CD103 Ber-ACT8, CD31 WM59, CD8 RPA-T8, CD147 HIM6, GITR 621, CCR4 205410, CD28 CD28.2, CD49d 9F10, CD62L DREG-56, CD3 UCHT1, CXCR3 G025H7, CD73 AD2, CD161 HP-3G10, CD39 A1, ICOS C398.4A, OX40 Ber-ACT35, CCR10 6588-5, CD137 4B4-1, CD27 L128, GARP 7B11, CD25 2A3, CD25 M-A251, CTLA4 14D3, CD4 SK3, CD38 HIT2, HLA-DR L243, CD71 OKT-9, and KLRG1 2F1/KLRG1 APC. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

A B-cell panel may include at least two of CD10, CD117, CD11c, CD127, CD16, CD179a, CD179b, CD19, CD20, CD21, CD22, CD23, CD235, CD24, CD27, CD33, CD34, CD38, CD40, CD43, CD45, CD45RA, CD49d, CD5, CD61, CD62L, CD66b, CD7, CD72, CD79b, CXCR4, HLADR, IgD, IgMi/IgMs (IgH), kappa, lambda, Pax5, PreBCR, RAG1, and TdT. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

A monocyte and MDSC panel may include at least two of CD14, CD16, HLA-DR, CD163, CD206, CD33, CD36, CD32, CD64, CD13, CD11b, CD11c, CD86, CD274, CCR2, CD163, CD13, CD123, and CD206. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

A DC panel may include at least two of EpCAM/CD326, CD19, CD117, CD11b, BDCA2/CD303, CD16, CD127/IL-7R, CD123/IL3R, CD66b, CD163, CD45, CCR7, CD14, CD11c, BDCA3/CD141, CD335/NKp46, BDCA-1/CD1c, CD1a, CD172a/b/SIRP alpha, HLADR, CD34, CD115/CSF1R, CX3CR1/CX3CR1, CD116/GMSFR, CD275/ICOSL, TLR4, CD274/PDL1, CLEC9A/DNGR1, CD135/FLT3, Dectin-1/CLEC7A, CD206/MMR, CD83, Langerin/CD207, CD45RA, CD33, CD2, CD81, CD5 (UCHT2), CD26, XCR1-PE, Anti-APC, Siglec-6/CD327 PE, CD100-APC, Ax1, CADM1/SynCAM, CD205/DEC205, BDCA2/CD303 APC, BDCA3/CD141 PECy7, BDCA4/CD304 APC, CD123 BUV395, CD16 BV650, CD163 FITC, CD19 PerCP/Cy5.5, CD20 PerCP/Cy5.5, CD1a Pacific Blue, CD2 APC/Cy7, CD206/MMR APC (dine 15-2), CD3 PerCP/Cy5.5, CD335 PerCP/Cy5.5, CD4 BV785, CD45 BV785, CD5 BV737, CD66b PerCP/Cy5.5, CD8a APC/Cy7, CD81 PerCP/Cy5.5, CLEC9A/DNGR1 APC, CD209/DC-SIGN APC, Dectin1/CLEC7A PE, HLADR BV605, Langerin/CD207 PE, TCF4/E2-2, Anti-fibroblast (TE7), CD140a, DARC, and Desmogelin-3. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein. In certain aspects, a panel may include element tagged oligonucleotide probes that hybridize (directly or through a hybridization scheme) to target RNA, such as RNAs encoding cytokines. For example, a panel may include two or more element tagged oligonucleotide probes to RNA encoding IFNg, IL-2, TNF, CXCL8, IL8, CC14, IL-1B, IL-6, CCL2, ILRN and Il1a. In some cases, the lyophilized panel can include at least 3, 4, 5, 6, 7, 8, 9, 10, 11, 12, 13, 14, 15, 16, 17, 18, 19, or 20, antibodies from this list. The panel may be provided in lyophilized form, or in a solution. The panel may be a distinct panel as described herein.

While numerous example lyophilized panels are described herein, other combinations of antibodies or other element-tagged moieties can be used to facilitate conducting a desired assay. In some aspects, one or more panels described above may be provided in non-lyophilized form, such as in a solution. Two or more of the above panels may be provided in mixture (in a joint panel).

Each of the lyophilized panels disclosed herein can be associated with a particular gating strategy used to identify certain traits based on the antibodies in the panel. To facilitate rapid analysis, the gating strategy associated with a particular panel can be pre-loaded into the software for analyzing the elemental analyzer data. Once selected (e.g., manually or automatically), the software can use that gating strategy to produce results from the elemental analyzer data. For example, using a human peripheral blood phenotyping kit, a pre-loaded gating strategy can help differentiate the different major peripheral blood cellular subsets based on the presence of the various antibodies (e.g., presence of the element tags associated with the antibodies) in the sample. In some cases, combination of multiple panels or panel subsets, such as those identified herein, can enable the use of new or additional gating strategies. For example, when combining a human peripheral blood phenotyping panel and a human hematopoietic stem and progenitor cell phenotyping panel, a new gating strategy can be used to help exclude lineage-positive cells from the results.

Within each panel, each antibody can be tagged with a unique element tag, such that a unique isotope or combination of isotopes is associated with each antibody in the panel. In some cases, especially for panels designed to be used in conjunction with another panel, the unique element tags from one panel are distinct from the unique element tags of the other panel. The mapping of each antibody to each isotope or combination of isotopes can be stored as an element tag mapping. This element tag mapping can be used by the software disclosed herein to interpret data received from an elemental analyzer.

In some cases, a lyophilized antibody panel can include one or more supplemental reagents. Such supplemental reagents can include any suitable lyophilizable reagent that is usable in conducting an assay using the antibodies of the lyophilized antibody panel. Examples of suitable supplemental reagents include red blood cell lysis, wash buffers, fixation reagents, permeabilization reagents, and the like.

The process of preparing a lyophilized panel can include obtaining antibodies, conjugating antibodies with their respective element tags, titrating the conjugated antibodies in a panel for quality control in liquid form, diluting the liquid antibodies in a stabilizer based on the titration results, combining the diluting antibodies with an excipient, combining the antibody and excipient mixtures together to form a single admixture, lyophilizing the admixture, then backfilling the lyophilized admixture and sealing it within a container. In some cases, individual antibody and excipient mixtures can be lyophilized prior to combining them together in admixture, although generally the various antibody and excipient mixtures are combined prior to lyophilization. Any suitable excipients can be used, such as sugars (e.g., trehalose, sucralose, and mannitol). The use of trehalose and/or sucrose can help inhibit protein unfolding while providing a glassy matrix. The use of mannitol can act as a bulking agent. In some cases, an excipient can include a sugar alone or in combination with bovine serum albumin (BSA). In some cases, the excipient can be a mixture of approximately 5%-20% (e.g., 10%) sugar in PBS and 5%-20% (e.g., 10%) BSA in PBS.

Lyophilization itself can occur in multiple stages, including a thermal stage, an evacuation stage, a drying stage, and a holding stage. During each stage, specific settings for temperature, ramping time, holding time, and/or vacuum (e.g., lyophilization settings) can be used to control the lyophilization equipment. The lyophilization equipment can be any suitable equipment for controlling the temperature and vacuum of the admixture to be lyophilized. In some cases, each stage can comprise multiple distinct sub-stages, each with distinct lyophilization settings. During a thermal stage, the temperature can be held somewhere between −60 and 0° C. for a duration while the vacuum is held in a range between 100-500 Torr. During an evacuation stage, the vacuum can be brought down to a range between 100-500 mTorr. During a drying stage, the temperature can be manipulated between ranges of −50 to 30° C. while the vacuum is held in a range of 10-150 mTorr. During a holding stage, the temperature can be maintained at a temperature between 10 and 30° C., while the vacuum is permitted to raise to a range between 100-500 mTorr. After the holding stage, the lyophilized admixture can be backfilled, which can include raising the pressure in the container, such as up to a range from 200 Torr up to 760 Torr, although in some cases, backfilling occurs at a pressure below ambient pressure. In some cases, during all stages of lyophilization, the temperature of the admixture does not rise above a glass transition temperature of the admixture.

During the lyophilization process, the moisture content of the admixture is reduced. In certain cases, the lyophilized panel can have a moisture content that is at or less than 5% by weight, although in some cases the moisture content can be at or less than 0.5% weight. In some cases, the lyophilized panel can have a moisture content by weight that is at or less than 5%, 4.9%, 4.8%, 4.7%, 4.6%, 4.5%, 4.4%, 4.3%, 4.2%, 4.1%, 4%, 3.9%, 3.8%, 3.7%, 3.6%, 3.5%, 3.4%, 3.3%, 3.2%, 3.1%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.95%, 0.9%, 0.85%, 0.8%, 0.75%, 0.7%, 0.65%, 0.6%, 0.55%, 0.5%, 0.49%, 0.48%, 0.47%, 0.46%, 0.45%, 0.44%, 0.43%, 0.42%, 0.41%, 0.4%, 0.39%, 0.38%, 0.37%, 0.36%, 0.35%, 0.34%, 0.33%, 0.32%, 0.31%, 0.3%, 0.29%, 0.28%, 0.27%, 0.26%, 0.25%, 0.24%, 0.23%, 0.22%, 0.21%, 0.2%, 0.19%, 0.18%, 0.17%, 0.16%, 0.15%, 0.14%, 0.13%, 0.12%, 0.11%, and/or 0.1%. In some cases, the lyophilized panel can have a moisture content that is at or greater than 0.05% by weight, such as between 0.05 and 1% by weight.

A lyophilized panel can be stored in any suitable container, such as a tube, a pouch, or wells of a well plate. In some cases, a single panel can be stored in a single container. In some cases, a single panel can be evenly dispersed throughout multiple containers, such as multiple wells of a well plate. In some cases, a lyophilized panel can be stored within an inert atmosphere (e.g., N₂) or within air (e.g., dry air). Lyophilized panels can be stored in hermetically sealed containers.

A lyophilized panel can be used similar to other antibody panels. The lyophilized panel can be re-suspended in solution before or after addition of the sample to be analyzed. In some cases, a sample of human peripheral blood can be combined with heparin prior to mixing with the lyophilized panel. In some cases, the blood sample can be mixed directly into the container containing lyophilized panel, although that need not always be the case. After mixing the sample with the lyophilized panel, the mixed sample can be incubated for a time, washed, and then interrogated using an elemental analyzer. In some cases, further staining can occur prior to interrogation, such as staining of intracellular targets, which may require the permeabilizing and fixing of the sample.

In certain aspects, a kit includes an element standard (e.g., provided in its own kit or alongside other reagents described herein such as a lyophilized panel and/or barcoded beads). The element standard may include microbeads that comprise known amounts of a plurality of different metal isotopes. In certain aspects, the element standard may comprise microbeads having different amounts of one or more metal isotopes. For example, element standard microbeads may comprise 2 or more, 3 or more, 4 or more, or 5 or more populations of microbeads, wherein each population comprises a similar amount of each of a plurality of metal isotopes but populations differ in the amount of at least one metal isotope. A similar amount of a given isotope within a population may mean the standard deviation in the amount of the given isotope (number of atoms of that isotope in a bead of the population) may be less than 10%, less than 25%, less than 50%, less than 100%, less than 200%, or less than 300% of the average amount of the given isotope for beads in that population. A population of beads may have at least one isotope present in a significantly different amount compared to each other population in the kit (e.g., the isotope that is significantly different compared a first other population may be different from the isotope that is significantly different from a second other population). For example, the difference between the average amount of an isotope between populations may be greater than 2 fold, greater than 3 fold, greater than 4 fold, or greater than 5 fold the standard deviation within one or both of the populations.

In certain aspects, element standard microbeads may comprise at least 2, 3, 4, 5, 6, 7, 8 or 9 variable isotopes that differ in amount between at least some populations of microbeads. The isotopes (e.g., variable isotopes) of element standard microbeads may cover a mass range of more than 10, more than 20, more than 30, more than 40, or more than 50 amu. At least one isotope may remain constant across all or some populations of microbeads, such that the ratio of the constant isotope(s) and variable isotopes may be used to indicate the bead population (e.g., to identify the expected amount of variable isotopes in the bead).

Microbeads may have a size larger than 100 nm and smaller than 1000 um, such as between 500 nm and 100 um. As such, microbeads may have more than a thousand, ten thousand, hundred thousand, or million atoms per bead. Microbeads may be made according to any of the methods described to other beads herein, such as a method of making a assay and/or sample barcoded bead (e.g., without the step of binding a biomolecule such as an SBP to the surface), or any suitable method.

The element standard may be useful for calibration of a mass cytometer and/or normalization of signal obtained across a sample run. For example, the element standard may be used to quantify antibody bound, such as antibody bound per cell. The element standard microbeads may be mixed with cells and run on a mass cytometer (e.g., such that microbead events and cell events are detected individually). As such, the mass of at least one isotope in microbeads may be different from any mass tag used for cells.

In certain aspects, a method includes calibrating a mass cytometer (at the start and/or during a sample run) based on an element standard (e.g., kit including an element standard) described herein. Alternatively or in addition, a method may include normalizing mass cytometry data based on an element standard (e.g., kit including an element standard) described herein. For example, the isotope signal from a cell (i.e., from a particular mass channel) may be normalized to the intensity of isotope(s) of similar mass and/or amount from microbead(s) acquired at a similar time. For example, the signal from a metal isotope tagged antibody bound to a specific analyte of a cell may be normalized to the intensity of an isotope signal most similar in mass and intensity and obtained within a time window of the cell event. A standard curve across different masses and/or intensities may be created to normalize signal from a cell event, based on element standard microbeads detected within a time window of that cell event. The time window may be less than 1000 seconds, less than 100 seconds, or less than 10 seconds. Cell events for which a element standard microbead of a population is not obtained within the time window may be discarded from the dataset. The cell event may be normalized based on the most recent microbead event (e.g., normalized based on signal provided by the most recent microbead event for each population of microbeads). In certain aspects, the same calibration curve may be used to calibrate all cell events within a time window.

In certain aspects, the amount (e.g., average number of atoms and optionally deviation) of each of a plurality of isotopes in an element standard microbead (e.g., element standard microbead population) may be known, and the amount (e.g., average number of atoms and optionally deviation) of atoms of a given isotope attached to an antibody bound to an analyte of the cell may both be known, and together used to calculate the antibody (e.g., to the analyte) bound per cell. For example, fractionation and analysis of labeled antibody may be performed by FPLC (e.g., size exclusion FPLC and/or anionic ion exchange FPLC) and the bulk amount of protein may be used to backcalculate antibody amound, and bulk metal may be determined by mass spectrometry. Element standard microbeads may be examined (e.g., by electron microscopy) to determine size and uniformity. Calibration of the instrument (e.g., across and/or between sample runs) may further enable quantitation of antibody bound.

In certain aspects, kits or methods with element standard microbeads may be combined with other aspects described herein, including lyophilized mixtures of metal tagged antibodies and/or assay barcoded beads. For example, element standard microbeads may be provided in the same kit or even container (e.g., in mixture with) lyophilized antibodies. Alternatively, cells stained with lyophilized antibodies described herein may later be combined with element standard microbeads prior to analysis by mass cytometery. Normalization (such as quantitation of antibody bound per cell) may improve the ability of gating cell populations, such as by an automated gating software described herein.

The following example procedure outlines steps for staining a sample of human peripheral blood. Sodium heparin salt is added at a final concentration of 100 U/mL to an aliquot of blood required for staining (10 uL of 10 KU/mL into 1 mL blood). This mixture is vortexed for two seconds and incubated for at least 20 min at room temperature. The antibody cocktail mix (e.g., lyophilized antibody panel) in 5 ml tubes is prepared. A volume of heparin-blocked blood is added directly into 5 mL tubes so that the final volume (blood+antibody cocktail) is 300 μL and the mixture is vortexed for 2 seconds to ensure that the lyophilized product is resuspended. The mixture is incubated for 30 minutes at room temperature. Immediately after staining is complete, add 250 μL of Cal-Lyse lysing solution to each tube. Incubate in the dark for 10 minutes at room temperature. Add 3 mL of water to each tube. Vortex tubes for two seconds and incubate for 10 minutes at room temperature. The cell suspension will begin opaque and become translucent after the 10 minutes incubation. If the cell suspension is not entirely translucent after 10 minutes, re-vortex the sample and incubate it at room temperature for an additional 5 minutes. Centrifuge the tubes and decant the supernatant into a 50 mL waste tube. Add 3 mL of cell staining buffer to each tube. Centrifuge the tubes and decant the supernatant. Repeat wash steps twice for a total of three washes. Visually inspect the cell pellet and the cell supernatant after each wash. If the cell pellet or supernatant is red, repeat washes until the supernatant is clear and the cell pellet is white. Next, the cells are fixed and DNA is intercalated. Prepare a fresh dilution of 16% formaldehyde to a 1.6% working solution with phosphate-buffered saline (PBS). Add 1 mL of 1.6% formaldehyde to each tube. Vortex the tubes for 2 seconds and incubate for 10 minutes at room temperature. Centrifuge tubes at 800×G for 5 min at room temperature and decant the supernatant. Dilute intercalator for a final concentration of 125 nM in a fixing and permeabilization buffer. Add 1 mL of the intercalator+buffer mixture to each tube. Vortex the tubes for two seconds, and incubate overnight at 4° C. Cells can be left at 4° C. in formaldehyde for up to 48 hours. Fixed cells can be seeded onto prepared glass slides if required or available. The cells are then washed prior to sample acquisition. Add 2 mL of cell staining buffer to each tube. Vortex the tubes for two seconds and centrifuge at 800×G for 5 min at room temperature. Decant the supernatant once centrifugation is complete. Repeat wash steps once for a total of two washes. Add 2 mL of a cell acquisition solution to each tube. Briefly vortex the tubes. Count cells to determine final the final volume of resuspension during acquisition. Centrifuge tubes at 800×G for 5 min at room temperature. Aspirate or pipette off the supernatant once centrifugation is complete. Keep cells pelleted and stored at 4° C. until ready to acquire. Create a mixture of 90% cell acquisition solution and 10% with four element calibration beads by volume in a 15 mL tube. Once ready for acquisition, resuspend cells in cell acquisition solution+four element calibration bead mixture at a concentration between 0.5×10⁶ cells/mL to 1.5×10⁶ cells/mL. Remove the cell-strainer caps from 5 mL polystyrene round-bottom tubes with a cell-strainer cap and place it onto a 5 mL polypropylene round-bottom tube. Filter the resuspended cells though the cell-strainer cap. Acquire samples on a pre-tuned ICP-MS instrument. Acquire samples at no more than 600 events/s. At least 400,000 events should be acquired.

As used herein, fixing and permeabilization refers to chemical cross-linking of cellular components by agents such as glutaraldehyde, formaldehyde, formalin, ethanol, methanol, etc., and creating holes in the cell membrane with detergents. Suitable detergents may be readily selected from among non-ionic detergents. Desirably, these detergents are used at a concentration between about 0.001% to about 0.1%. One detergent that may be used is Triton X-100 (Sigma T9284). Examples of other suitable detergents include Igepal and Nonidet P-40. Other suitable detergent may be readily selected by one of skill in the art.

Certain aspects of the present disclosure are useful for analyzing whole blood, such as human peripheral blood. In some cases, whole blood can be separated into PBMCs and isolated plasma prior to staining with the lyophilized panel. The lyophilized panel can stain PBMCs. In some cases, a sample or PBMCs from a sample can be tagged with a sample barcode as disclosed herein, whether before or after staining by the lyophilized panel. When tagged, the samples can be pooled together prior to interrogation by an elemental analyzer. The stained PBMCs can be interrogated using an elemental analyzer, such as a mass analyzer.

The data from the elemental analyzer can include data indicating the presence of various isotopes in a sample. Specifically, this data can include the presence of the detectable isotopes of the element tags that remained bound to the sample after washing the sample. The elemental analyzer can interrogate the sample on a cell-by-cell or particle-by-particle basis, thus generating data on a cell-by-cell or particle-by-particle basis. For example, data from an elemental analyzer can indicate the presence of various isotopes for each cell or each particle of a sample.

During an automated analysis process, software can decode the elemental analyzer data into useful and readable results. This software can be incorporated into the elemental analyzer or provided separate (e.g., on a separate computing device). Decoding the elemental analyzer data can result in identifying the various element tags that were detected by the elemental analyzer, which can be known as element tag data. Each element tag can be associated with a particular marker, such as an antibody, a live cell marker, or a sample barcode. These associations can be stored as an element tag mapping accessible to the software. The software can then apply this element tag mapping to the element tag data to generate useful and readable results. In some cases, the software can select (e.g., manually or automatically) a particular gating scheme to use in interpreting the element tag data. For example, for a particular lyophilized antibody panel, a particular gating scheme can be used to help interpret the element tag data such that the presence or absence of various antibodies from the lyophilized antibody panel can help identify a particular cell's type (e.g., Activated T Helper Cells, Memory T Helper Cells, or Memory B Cells). The software can then output appropriate results, such as to a display or storage file.

In some cases, the software can automatically identify the cell types of the sample. The automatic identification of cell types can be based on a predetermined gating of cell populations sharing similar expressions of a subset of surface markers. Alternatively or in addition, identification of cell types can also be guided by a clustering algorithm.

In some cases, the software can output cell type results. Cell type results can include relative quantification (e.g., % of total cells, % of parent cells (parent cell population), % of grand-parent cells, and the like) for various cell types, such as CD4 αβ T cells (e.g., Total CD4, Naïve, Central memory, Effector, Effector memory, and Regulatory); CD8 αβ T cells (e.g., Total CD8, Naïve, Central memory, Effector, Effector memory); δγ T cells; B cells (e.g., Total B cells, Naïve, Memory, Resting memory, Transitional); NK cells; Monocytes; and/or Dendritic cells.

In some cases, the software can output marker intensities. Marker intensities can include intensities (e.g., median intensity) for markers of each cell type and/or any other marker at the user's discretion. For example, a visual output can be generated showing the intensity “color” for each cell type in a phenotypic tree. In another example, a comparison of each result to a user-generated data set of reportables containing frequencies, intensities, and cell counts, which can be shown with a visual display (e.g. if the median is 1.5 sigma higher than normal, the color can be visually “hot”; if the median is 1.5 sigma lower than normal, the color can be visually “cool”).

In some cases, the software can output other results, such as dot-plots of any two markers and/or histograms of any marker. In some cases, the software can automatically flag any markers having a distribution with more than one mode (e.g., a bimodal distribution). In some cases, the software can output a report text file with desired reportables (e.g., frequencies, intensities, and cell counts). In some cases, the software can maintain a database of reportables (e.g., frequencies, intensities, and cell counts). In some cases, the software can output a print-quality formatted report with user-selected plots and tables.

Certain aspects of the present disclosure relate to the barcoding of samples on a per-sample and optionally per-assay basis using element tags. As disclosed herein, element tags can be unique and distinguishable based on their isotopic composition. For example, an element tag (or combination of element tags) used in a sample or assay barcode can have a unique isotope or unique combination of isotopes that distinguishes it from other element tags. Thus, when the assay barcode is identified during elemental analysis of the sample, an inference can be made that the particular cell or particle being interrogated has undergone the assay (e.g., treatment/stimulation conditions, staining with a particular panel of element tagged SBPs, etc.) associated with the assay barcode. Of note, assay and/or sample barcodes can be used to identify (and throw out data from) particle doublets that comprise two or more assay barcodes, or two or more sample barcodes.

Assay barcoding can be achieved by binding analytes in a sample to an assay barcode that is associated with a particular target analyte being detected. The assay barcode may comprise a solid support (e.g., assay bead), a capture biomolecule (e.g, an assay biomolecule that specifically binds a target analyte in a sample, and binds it to the surface of an assay bead), and a distinguishable combination of isotopes (assay barcode) associated with that target analyte. The solid support may be a planar surface/slide (e.g., comprising assay barcoded array), or assay barcoded bead(s). The assay biomolecule may be an oligonucleotide (e.g., that specifically hybridizes to a target RNA or DNA), an affinity reagent (e.g., an antibody, aptamer or lectin), or a substrate (e.g., a peptide comprising an element tag that is cleaved in the presence of a target enzyme, such as a target protease). The assay biomolecule may be bound to assay beads, such that different assay biomolecules (e.g., that bind to different target analytes) are bound to beads that have different assay barcodes. A reporter may provide detection of the assay biomolecule, as described herein. Such reporter may include a reporter biomolecule (e.g., oligonucleotide or antibody) that binds (directly or indirectly) to the target analyte. A reporter may further include an element tag for detecting presence of the target analyte associated with the assay bead (e.g., associate by binding to an assay biomolecule that is itself bound to the bead).

Together, the assay biomolecule and reporter may provide increased specificity, as both need to bind the target analyte to provide a signal. Further, the analyte bound by the assay biomolecule will be presented on the surface (or spaced from the surface) of the bead, such that a reporter with a bulky element tag can still bind to the target. As such, signal amplification methods described herein may be used to detect target analyte.

As used herein in the context of mass cytometry, signal amplification is the association of more than 30, more than 50, more than 100, more than 200, or more than 500 labeling atoms (e.g., of an enriched isotope) with a target analyte (i.e., a single instance of the target analytes bound by a specific binding partner). In certain aspects, labeling atoms may be heavy metals, such as lanthanides or transition metals. In certain aspects, signal amplification may be performed for more than 2, 5, 10 or 20 target analytes. In certain aspects, signal amplification may include use of branched conjugation of a mass tag to a biomolecule, a high sensitivity polymer, a large mass tag particle, a mass tag nanoparticle, a hybridization scheme associating a plurality of element tagged oligonucleotides (e.g., comprising multiple instances of the same element tag) with a target, and/or enzymatic deposition of a plurality of element tags. In certain aspects, signal amplification uses a mass tag polymer as described herein.

Mass tagged oligonucleotides may be hybridized, directly or indirectly, to a target oligonucleotide. For example, one or more intermediate oligonucleotides may provide a scaffold on which a plurality of mass tagged oligonucleotides can hybridize, thereby amplifying signal. Aspects of the subject application therefore include oligonucleotides for hybridization based signal amplification.

The target oligonucleotide may be a DNA or RNA molecule (such as coding RNA, small interfering RNA, or micro RNA) endogenous to a cell. The target oligonucleotide may be single stranded. The target oligonucleotide may have a known specific sequence (or homology to a known specific sequence). In certain aspects, a biomolecule, such as an antibody or derivative thereof may be conjugated to the target oligonucleotide, such as to a synthetic single stranded DNA oligonucleotide comprising a known sequence. In such cases, both the antibody and the oligonucleotides may be referred to as biomolecules.

After binding of the biomolecule to analyte in a sample, a plurality of mass-tagged oligonucleotides may be hybridized, directly or indirectly, to the first oligonucleotide. The hybridization may be branched or linear. In certain aspects, a polymerase may extend the first oligonucleotide along a template to provide additional sites for attachment of an element tag (such as additional hybridization sites for an element tagged oligonucleotide). Mass tagged oligonucleotides may include a single labeling atom, or may include a polymer comprising multiple labeling atoms. Mass tagged oligonucleotides may include a labeling atom, such as a heavy metal atom, in the chemical structure of the oligonucleotide itself.

Signal amplification may uniquely benefit bead based assays, in which the same reporter tag (labeling metal element or isotope) can be amplified and used across different beads and their target analytes. In certain aspects, a mass tagged oligonucleotide may be mass tagged with a high sensitivity polymer or a nanoparticle described herein in addition to being used in a signal amplification hybridization scheme.

In certain aspects, assay beads may be customizable such that a user may assay biomolecules of interest to each assay barcoded bead. This attachment may be by a chemical bond, or may be by addressing assay biomolecules to specific assay barcoded beads in the same mixture. This addressing may be performed by providing assay barcoded beads that present an oligonucleotide sequence unique for each assay barcode, and the user can attach a different addressing oligonucleotide to different biomolecules to attach to the bead surface, wherein an addressing oligonucleotide specifically hybridizes to one of the unique oligonucleotide sequences.

In cases where the assay biomolecule is an oligonucleotide, an element tagged reporter oligonucleotide may hybridize to another portion of the target RNA or DNA, thereby providing a signal when the target RNA or DNA is bound to the bead. In cases where the assay biomolecule is an affinity reagent, such as an antibody that binds an analyte at a first epitope, an element tagged reporter affinity reagent (e.g., reporter antibody) may bind to another epitope on the analyte, thereby providing a signal when the target analyte is bound to the bead. The analyte may further be bound by a reporter, such as an element tagged reporter antibody or oligonucleotide. The reporter may comprise a high sensitivity (e.g., intensity) element tag that provides a highly abundant isotope (e.g., more than 50, 100, 200, 500, 1000 copies of a single isotope), thereby enabling detection of a smaller number of a target analyte bound to an assay bead. Such a high sensitivity element tag may include a nanoparticle (e.g., comprising a metal nanocrystal surface functionalized to bind biomolecule such as an antibody or oligonucleotide) or a hyperbranched polymer. For example, multiple reporter biomolecules comprising the same nanogold particle element tag would provide a high signal and take advantage of the fact that separate reporter biomolecules comprising the same element tag can be distinguished by the assay barcode (e.g., of the bead presenting the analyte they are specific to). In certain aspects, a nanoparticle tag (e.g., gold nanoparticle) may be associated with a reporter probe through a biotin-avidin (e.g., biotin-streptavidin) interaction. For example, the nanoparticle (e.g., gold nanoparticle) may be conjugated to streptavidin. The reporter may also comprise a low sensitivity element tag that provides a low abundance isotope (e.g., less than 100, 50, 30, 20, 10, or 5 copies of an isotope) that is distinct from the highly abundant isotope, thereby allowing detection/quantitation of the amount of an analyte that is such high abundance that the highly abundant isotope would saturate the detector. In certain aspects, the high and low abundance isotopes have a difference in mass (e.g., greater than 5, 10, 20, 30, 40 or 50 amu) such that saturation of the detector by the high abundance isotope does not affect detection of the low abundance isotope. Reporters for different analytes (e.g., comprising antibodies that bind to target analytes of different assay beads) may comprise the same isotopes or combination of isotopes, since the analytes will be distinguished by the unique assay barcodes of the beads.

In certain aspects, a reporter may include a reporter system that provides signal amplification through association of a plurality of instances of an element tag with a single instance of the target analyte. Signal amplification may be by enzymatic deposition, hybridization (e.g., branched hybridization, chain hybridization, and/or hybridization of a plurality of reporter oligonucleotides to a single long intermediate oligonucleotide), extension (e.g., a single extension, rolling circle extension), and/or a series of branched conjugations. In certain aspects, a plurality (e.g., all) of the analytes detected with the assay beads may be detected with the same reporter system. In certain aspects, a signal amplification reporter system may have a high sensitivity element tag.

For example, an element tag comprising an enzyme substrate moiety may be deposited from solution onto the bead (or a molecule attached to the bead) by an enzyme attached to a reporter biomolecule. Such a reaction may be covalent binding by a tyramide element tag acted on by horse radish peroxidase bound to a reporter biomolecule.

Aspects include a hybridization scheme, such that a plurality of element tagged oligonucleotides hybridize indirectly (through one or more oligonucleotide intermediates) to a single oligonucleotide target. For example, the oligonucleotide target may be a target RNA or DNA (e.g., gDNA or cDNA) sequence, or may be an oligonucleotide present on a reporter antibody.

As described herein, mass cytometry enables enough detection channels (mass channels) to detect both a sample and assay barcode in beads while allowing an additional channel for a reporter (e.g., for detecting the assay target). As such, the bead assays described herein may be sample and/or assay barcoded for use in mass cytometry. For example, a plurality of different conditions (e.g., drug candidates such as enzymes or an agonist or antagonist of one or more enzymes) may be applied to a biological sample, and its effect on a plurality of targets may be detected with enzyme assay beads. Individual conditions may be identified with a sample barcode shared across different assay beads exposed to the same conditions. Assay barcoded beads may be combined prior to analysis, such as before exposure to a condition. Sample barcoded beads may be combined prior to analysis.

In certain aspects, the enzyme may be a protease, kinase, phosphatase, or a DNA modifying protein such as a DNA methyltransferase. The target may be the substrate acted on by the enzyme, and the reporter (e.g., a reporter biomolecule as described herein) may only bind the target before or after it is acted on by the enzyme. For example, a phospho-specific antibody that detects the phosphorylated form of a protein target, which may be increase in abundance when acted on by a kinase enzyme or decreased in abundance when acted on by a phosphatase. When the enzyme is a protease, a reporter may be associated with the end of a peptide substrate and removed from association with the bead when the substrate is cleaved (such that a decrease in the reporter element tag indicates an increase in protease activity).

Sample barcode may be used to indicate which of a number of enzymes (or agonists/antagonists thereof) was tested in a particular assay. For example, the candidate enzyme, agonist, antagonist, may be added to a biological fluid such as a cell lysate, after which the sample is contacted with assay barcoded beads to detect activity of the enzyme. Alternatively, the candidate may be administered to cells (e.g., directly or by genetic engineering) or to an organism such as a patient or mammalian test subject, and a sample taken from that source may be contacted with the assay beads. Sample barcoding allows many such candidates to be screened in parallel. In either case, the sample barcode can be added as described for beads herein to identify the candidate. For example, more that 10, more than 20, more than 50, more than 100, more than 500, or more that 1000 distinct samples can be barcoded. For example, 12 distinct isotopes in unique combinations of 6 provides 924 distinct combinations (e.g., for barcoding of up to 924 samples). Another 12 distinct isotopes could be used to barcode close to 1000 assays. As such, more than 10, more than 20, more than 50, more than 100, more than 500, or more that 1000 distinct assay beads can be barcoded (e.g., beads detecting amount of a different substrate acted on by the candidate). At least one channel would be left for detection of the substrate by a reporter, as described herein. This may allow unprecedented screening with an immediate readout by mass cytometry.

Post-translational modifications of proteins are carried out by enzymes within living cells. Known post-translational modifications include protein phosphorylation and dephosphorylation as well as methylation, prenelation, sulfation, and ubiquitination. The presence or absence of the phosphate group on proteins, especially enzymes, is known to play a regulatory role in many biochemical pathways and signal transduction pathways.

Bead based kinase assays for mass cytometry are discussed in US patent publication US20070190588, which is incorporated by reference and summarized below. However, such bead based assays have not been proposed for both sample and assay barcoding, which provides advantages for screening and is uniquely enabled by the high plexity of mass cytometry.

A kinase function is to transfer phosphate groups (phosphorylation) from high-energy donor molecules, such as ATP, to specific target molecules (substrates). An enzyme that removes phosphate groups from targets is known as a phosphatase. The largest group of kinases are protein kinases, which act on and modify the activity of specific proteins. Various other kinases act on small molecules (lipids, carbohydrates, amino acids, nucleotides and more) often named after their substrates and include: Adenylate kinase, Creatine kinase, Pyruvate kinase, Hexokinase, Nucleotide diphosphate kinase, Thymidine kinase.

Protein kinases catalyze the transfer of phosphate from adenosine triphosphate (ATP) to the targeted peptide or protein substrate at a serine, threonine, or tyrosine residue. Protein kinases are distinguished by their ability to phosphorylate substrates on discrete sequences. Commercially available kinases can be in the active form (phosphorylated by supplier) or in the inactive form and require phosphorylation by another kinase.

A protein phosphatase hydrolyses phosphoric acid monoesters at phosphoserine, phosphothreonine, or phosphotyrosine residue into a phosphate ion and a protein or peptide molecule with a free hydroxy group. This action is directly opposite to that of the protein kinase. Examples include: the protein tyrosine phosphatases, which hydrolyse phosphotyrosine residues, alkaline phosphatase, the serine/threonine phosphatases and inositol monophosphatase.

Another aspect is to provide a method for a phosphatase assay, comprising: incubating, in a multitude of solutions, each solution comprising a different free phosphorylated substrate labeled with an element tag, which can optionally be the same element tag for all substrates, a plurality of element labeled supports having attached thereto a metal ion coordination complex, in such manner that each type of phosphorylated substrate labeled with an element tag, which can optionally be the same element tag for all substrates, is attached to a single type of element labeled support; separating free phosphorylated substrate from the bound substrate attached to the metal ion coordination complex attached to the multitude of element labeled supports in the multitude of separate solutions; incubating the multitude of element labeled supports having attached thereto the multitude of phosphorylated substrates labeled with an element tag, which can optionally be the same element tag for all substrates, through attachment to the metal ion coordination complex that is attached to the supports in a single solution with ADP and at least one phosphatase in conditions that enable the phosphatase to dephosphorylate the phosphorylated substrates; separating free non-phosphorylated substrate from bound phosphorylated substrate labeled with an element tag, which can optionally be the same element tag for all substrates, attached to the metal ion coordination complex attached to said multitude of element labeled supports; and performing particle elemental analysis of bound phosphorylated substrate labeled with an element tag, which can optionally be the same element tag for all substrates, attached to the metal ion coordination complex attached to the multitude of element labeled supports.

Another aspect of the applicant's teachings is to provide a kit for the detection and measurement of elements in a sample, where the measured elements include an element tag attached to a phosphorylated substrate, an element of a metal ion coordination complex, and elements of uniquely labeled supports, comprising: an element tag for directly tagging phosphorylated substrate; a multitude of phosphorylated substrates; uniquely labeled supports; metal ion coordination complex; and optionally, phosphatase, phosphatase buffer and ADP.

Another aspect of the applicant's teachings is to provide a method for a kinase assay, comprising: incubating ATP, at least one kinase, a free metal ion coordination complex, and a multitude of non-phosphorylated substrates immobilized on element labeled supports in such manner that a single type of non-phosphorylated substrate is attached to a single type of element labeled support, in conditions to enable the kinase to phosphorylate the substrates; separating the multitude of phosphorylated substrates immobilized on element labeled supports having attached metal ion coordination complex from the free metal ion coordination complexes and the multitude of immobilized non-phosphorylated substrates; and measuring the multitude of phosphorylated substrate immobilized on element labeled supports having attached metal ion coordination complex by elemental analysis.

Another aspect of the applicant's teachings is to provide a kit for the detection and measurement of elements in a sample, where the measured elements include an element tag attached to a non-phosphorylated substrate and a metal ion coordination complex, comprising: an element tag for directly tagging non-phosphorylated substrate; non-phosphorylated substrate; a solid support; a metal ion coordination complex; and optionally, kinase; kinase buffer; and ATP.

The supports may be a coded bead that is sample and barcoded, as discussed herein.

Owing to their role in maintaining animal homeostasis, the assay and pharmacological regulation of enzymes have become key elements in identifying possible therapeutic agents. Proteases are a subclass of protein-degrading enzymes that have recently been shown to play a vital role in signaling pathways, the dysregulation of which can result in cancer, cardiovascular disease, and neurological disorders. Of the approximately 400 known human proteases, dozens are being studied as potential drug candidates. Small-molecule inhibitors of proteases are now considered valuable therapeutic leads for the treatment of degenerative diseases, for the treatment of cancer, and as antibacterials, antivirals and antifungals.

Bead based protease assays for mass cytometry are discussed in US patent publication US20170023583, which is incorporated by reference and summarized below. However, such bead based assays have not been proposed for both sample and assay barcoding, which provides advantages for screening and is uniquely enabled by the high plexity of mass cytometry.

There is a need for a robust, sensitive, and quantitative enzyme assay that allows for simultaneous measurement of multiple enzymatic reactions. Such an assay can allow for conservation of valuable biological sample and reagents, achieve high-throughput and reduced assay time, and decrease the overall cost of enzyme analysis.

One aspect of the invention is a method for detecting protease activity in a biological fluid. The method may include attaching a coded bead to a first amino acid of a peptide substrate to form an immobilized peptide substrate, the peptide substrate comprising a first amino acid and a last amino acid and being a substrate for a protease enzyme: attaching an element tag to the last amino acid of the peptide substrate to form a tagged peptide substrate: incubating the immobilized, tagged peptide substrate with the biological fluid: and detecting the element tag and the coded bead in the biological fluid by elemental analysis. The coded bead may be both assay and sample barcoded, as discussed herein.

A protease assay kit may include an assay coded bead attached to a first amino acid of a peptide substrate (an immobilized peptide substrate), the peptide substrate may include a first amino acid and a last amino acid and may be a substrate for a protease enzyme. An element tag may be attached at or near the last amino acid of the peptide substrate to form a tagged peptide substrate. The coded bead may be both assay and sample barcoded, as discussed herein.

A mixture of assay beads may together target at least 5, 10, 20, 50, 100, 200, 500, 1000 or more analytes. In certain aspects, sample barcodes may distinguish assay barcode beads and/or cells from at least 5, 10, 20, 50, or 100 or more different samples.

In certain aspects, the number and/or size of assay barcoded beads may vary by analyte they are specific for. For example, an assay barcoded bead that specifically binds a high abundance target analyte may have fewer antibodies on its surface, or be greater in number, than an assay barcoded bead that specifically binds a lower abundance target analyte in the same sample. For example, in a mixture of assay barcode beads, an assay barcoded bead specific for a first target analyte may be in 2, 5, 10, 20, 50, or 100 times as abundant as an assay barcode bead specific for a second target analyte. When the first analyte is in greater abundance than the second analyte, it will be diluted across the high number of assay barcoded beads that bind it. An assay barcode kit may comprise one or more assay barcode beads and/or reporters as described herein.

Per-sample barcoding can be achieved by tagging a sample with a sample barcode that is associated with that particular sample. Thus, when the sample barcode is identified during elemental analysis, an inference can be made that the particular cell or particle being interrogated is part of that particular sample. Thus, multiple samples can be pooled together at any time between applying the sample barcodes and interrogation using elemental analysis without losing the ability to associate interrogated cells or particles with their respective samples. By permitting pooling together of samples, the elemental analyzer can operate with a much higher throughput and the handling and storage of samples can be simplified.

Sample barcodes can include element tags that are coupled to element-tagged moieties (e.g., assay barcoding reagents or sample barcoding reagents) having targeting functions for all, a supermajority, or a majority or all cells or particles of a sample. As used herein, the term supermajority can include at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the cells or particles of a sample.

In some cases, a barcoding reagent can be a bead that is capable of containing or coupling to an element tag. In some cases, a unique assay barcode can be located within the bead and the surface of the bead can contain, be coupled to, or be functionalized to couple to a unique sample barcode. Any suitable bead for containing or coupling to element tags can be used, such as a polymer-assisted surface functionalized bead. Such a polymer may be poly-L-lysine, PEG (polyethylene glycol), PEG MEA (methyl ether acrylate), PVMS (polyvinylmethyl siloxane), polydopamine, polystyrene and/or another polymer or derivative known by one of skill in the art. In some cases, the bead can be functionalized with a first functional group for binding a moiety having a targeting function (e.g., an antibody) and a second functional group for binding a sample barcode. In some cases, however, the bead can be functionalized with only a single functional group capable of both binding a moiety having a targeting function and binding the sample barcode. In some cases, blocking agents (e.g., albumin) can be used to control attachment of moieties and sample barcodes to a bead. In some cases, a reactive functional group (e.g., a thiol, amine, thiol-reactive, amine-reactive, or click chemistry functional group, or even a highly reactive functional group such as isothiocyanate) can be used to facilitate labeling beads blocked with blocking agents, provided the functional group reacts with the surface of the cell or the cell is permeabilized beforehand. Such functional groups can be located on the sample barcodes to facilitate tagging of the barcoding reagents or samples themselves. Alternatively, free metal (e.g., a set of sample barcode isotopes) may be provided in solution may be bound by chelation groups on the surface of the bead. In certain aspects, beads may be functionalized to bind (or be bound by) the same sample barcode reagent as is used to barcode cells in an assay. In certain aspects, the combination of isotopes in a sample barcode may be the same for beads and cells from the same sample. In some cases, the assay beads may be blocked (e.g., with a blocking reagents such as BSA). In such cases, the sample barcode may bind to the blocking reagent(s).

Sample barcoding reagents for cells may include an element-tagged antibody or antibodies (that bind across a plurality of cell types or majority of cells in the sample), a element tag functionalized to bind non-specifically to cells (e.g., through a covalent interaction), and/or metal in solution. Sample barcoding reagents for cells may further include a reagent for bringing the sample barcode into the cell (e.g., DMSO, cell permeabilization reagents such as a detergent or alcohol, etc.). Sample barcoding reagents for assay barcoded beads may be present within the beads, on the surface of the beads, or may be applied to the beads. If for application to beads, the sample barcoding reagent may comprise functional groups as described herein to bind to the surface of the bead (e.g., to bind to functional groups presented by the bead or to a blocking reagent present on the bead surface). Sample barcoding reagents for a given sample may comprise a unique combination of isotopes specific to that sample. In certain aspects, cells and assay barcoded beads from the same sample (e.g., an individual blood sample) may be labeled with the same assay barcode. The same assay barcode used for labeling of cells and beads may comprise the same combination of isotopes and/or same means of attachment (e.g., functional group).

Sample barcoding reagents may be provided in admixture or alongside with a lyophilized antibody panel. Sample barcoding reagents may be provided in admixture or alongside with assay barcoded beads. Assay barcoded beads may be provided in admixture or alongside a lyophilized antibody panel. Assay barcoded beads and sample barcoding reagents may be provided in admixture with or alongaside a lyophilized antibody panel (e.g., where the sample barcoding reagents bind both assay barcoded beads and cells in the sample). In certain embodiments above, sample barcoding reagents may be in, on, or provided alongside assay barcoded beads.

In some cases, a highly reactive functional group (e.g., a thiol, amine, thiol-reactive, amine-reactive, or click chemistry functional group) can be used to facilitate labeling of cells with sample barcode. Such functional groups can be located on the sample barcodes to facilitate tagging of the barcoding reagents or samples themselves. Alternatively, free metal (e.g., a set of sample barcode isotopes) may be provided in solution may administered to the cell (e.g., in the presence of an reagent such as an alcohol, detergent and/or DMSO, to allow entry of the metal into the cell).

In some cases, the element tags used for assay barcodes and sample barcodes can include elements and/or isotopes not commonly used with element tagged lyophilized panels, or outside the lanthanide family. For example, assay barcodes and/or sample barcodes can be barcoded using Pd or Te. In some cases, an element tag for assay barcodes and/or sample barcodes can be a cadmium element tag containing cadmium isotopes chelated to a polymer (e.g., chelated to DOTA groups on a polymer) or bound to a cadmium binding protein.

In some cases, a kit can be provided containing barcoding reagents, such as beads, in a single container and multiple separate containers each containing unique sample barcodes. The barcoding reagents may or may not include assay barcodes. A user can combine separate volumes of these barcoding reagents with the different samples being assayed. Then, each sample can be combined with a unique sample barcode, permitting that sample barcode to bind to the barcoding reagents already in or bound to the sample and/or to cells or particles of the sample itself. After washing, this sample-barcode-tagged sample will comprise barcoding reagents, with or without assay barcodes, that all include the same sample barcode. Thus, this sample barcode can be used to identify cells or particles of this particular sample. The same process can be performed with other samples and other sample barcodes, thus resulting in a collection of multiple sample-barcode-tagged samples, with each sample being tagged with a unique and distinguishable barcode. Optionally, in some cases, sample barcodes can be used without separate barcoding reagents when the sample barcode can itself directly target the cells or particles of the sample.

In some cases, the association between a particular sample and its sample barcode can be stored, such as in a mapping. This sample barcode information can be accessed by interrogation or analysis software to automatically attribute elemental analyzer data or further results to the appropriate sample, based on the detected presence of the sample barcodes.

In some cases, a reporter reagent may be provided alongside an assay reagent and/or used to detect the presence of an analyte bound to the assay reagent. A reporter reagent may directly bind (specifically or non-specifically) to analytes in the sample before or after the analytes are bound by assay beads. A reporter reagent may include a biomolecule such as an oligonucleotide (e.g., that hybridizes a target RNA or DNA bound to an assay barcoded bead) or affinity reagent (e.g., an antibody, lectin, or aptamer). Such a reporter biomolecule, can be used to help identify the presence of a barcoding reagent (e.g., bead) and/or target particle during interrogation. A reporter reagent can include an element tag that is common to all reporter reagents for the same object (e.g., all reporter reagents for beads or all reporter reagents for a particular target analyte), but distinguishable from other element tags (e.g., sample barcoding element tags). Each reporter reagent can be associated with a particular object, such as a barcoding reagent (e.g., bead) or a target analyte. The reporter reagent can include antibodies or other moieties having a targeting function for their associated object. Thus, when a reporter reagent is mixed with other reagents (e.g., barcoding reagents) or a sample, the reporter reagent can tag their associated object with the element tag for that reporter reagent. When that element tag is detected during interrogation by elemental analysis, an inference can be made that the particle being interrogated is of the type associated with that particular reporter reagent. For example, if an element tag associated with a reporter reagent for a barcoding bead is detected, it can be assumed that the object being detected at that time, and thus the elements detected within a certain window before and after that time, are associated with that barcoding bead (e.g., are isotopes associated with an element tag within or on that bead). As used herein, a reporter reagent for a cell type can be an antibody form a lyophilized antibody panel.

A reporter biomolecule may comprise an element tag (e.g., a collection of one or more isotopes on a polymer chain or embedded within or on a bead). In some cases, the reporter reagent (such as a reporter antibody) can be specific to a free analyte that is associated with a particular assay reagent (e.g., bound by an assay antibody present on the surface of an assay reagent such as a bead). The reporter antibody and the assay antibody (e.g., the antibody coupled to an assay bead) can be the same type of antibody or can be different types of antibodies. In some cases, if the reporter antibody is different from the assay antibody (e.g., if polyclonal antibodies are used), the reporter antibody can be selected to not interfere with the binding of the assay antibody to the target analyte. In some cases, reporter reagent and assay reagent can be created from the two different antibodies (e.g., monoclonal antibodies), although that need not always be the case.

When multiple different assay reagents are used, with multiple target analytes, multiple sets of reporter reagents can be created and/or used, with each set containing the same element tag(s) (e.g., element tags that are not isotopically distinct), but a different reporter antibody coupled to the element tag(s). Thus, while the various reporter antibodies of the sets of reporter reagents may target various different target analytes, they will all tag those various target analytes with the same element tag associated with all reporter reagents. Alternatively or in addition, a reporter reagent may comprise both a high sensitivity and a low sensitivity element tag, as described further herein.

In an example, a kit can be provided with a set of 10-12 different barcoding reagents can be provided each having a unique assay barcode, a set of 6 different element tags can be separately provided for later combination with the barcoding reagents and/or samples, a barcoding reagent reporter (e.g., a reporter reagent targeting the barcoding reagents) can be provided included with or separate from the barcoding reagents and having its own unique element tag, and a set of 30-40 cell reporters (e.g., reporter reagents each targeting a specific cell type) can be provided with each cell reporter having a unique element tag. The number of each reagent, barcode, and/or reporter can be adjusted as desired.

In some cases, barcoding reagents and/or barcodes can be lyophilized and included in a lyophilized panel, such as the lyophilized panels described herein.

In some cases, barcoding reagents can be provided in a pre-configured form by preparing the barcoding reagents with a number of unique combinations of assay barcodes and sample barcodes. In such cases, each unique barcoding reagent can be stored in distinct containers, such as distinct wells of a well plate. In an example, a well plate can be established such that all wells along a particular column (or row) share the same assay barcode, whereas all wells along a particular row (or column) share the same sample barcode. In another example, a well plate can be established such that each filled well contains barcoding reagents with various combinations of a particular unique sample barcode and numerous assay barcodes. Thus, a first well may contain barcoding reagents all having a first sample barcode but each having different assay barcodes, and a second well may contain barcoding reagents all having a second barcode but each having different assay barcodes. In some cases, pre-configured barcoding reagents can require the manufacture of thousands of groups of unique beads.

In some cases, barcoding reagents (e.g., beads) can be provided in a semi-configured form by preparing the barcoding reagents with unique assay barcodes and a surface functionalized to bind a sample barcode. In such cases, each group of barcoding reagents can be coupled to a moiety (e.g., antibody) having a targeting function associated with the assay that is associated with the assay barcode of that group of barcoding reagents.

When semi-configured barcoding reagents are provided, the sample barcodes can be bound to the barcoding reagents before combining the barcoding reagents with samples. In an example, the different barcoding reagents can be mixed together and then placed across a set of containers (e.g., wells in a well plate). Then, unique sample barcodes can be added to each of the containers, the result of which can be mixed with a unique sample to perform assay-barcode-identifiable assays on that sample and simultaneously tag that sample with the sample barcode.

When semi-configured barcoding reagents are provided, the sample barcodes can be bound to the barcoding reagents after combining the barcoding reagents with samples. In an example, the semi-configured barcoding reagents can be provided together or otherwise mixed together. Then, the barcoding reagents can be added to each of a set of samples. Separately, before or after the barcoding reagents are added, a unique sample barcode can be mixed with each of the set of samples. The sample barcode can tag the barcoding reagents and/or the cells or particles of the sample.

In an example case, barcoding reagents can include assay barcoded beads functionalized with polydopamine for the attachment of capture antibodies. Another molecule (e.g., avidin) can be added alongside the capture antibodies. After capture antibodies are added to the assay barcoded beads, the beads could be mixed and split into an aliquot for each sample. For the sample barcode, a unique combination of element tags functionalized (e.g., with biotin) to bind the molecule can be added.

In some cases, a sample barcode can be coupled to a reporter to enable that reporter to function both as a reporter and a sample identifier. For example, an antibody for reporting a particular cell type can also function to identify the sample to which that cell type is associated. In such a case, multiple copies of the antibody and its unique element tag can be created, with each copy receiving a unique sample barcode. Then, each copy can be provided to each of multiple samples. After washing and interrogation, detection of a unique sample barcode can indicate presence of a particular sample and detection of the unique element tag for that antibody can indicate presence of its target in that sample. In some cases, multiple antibody panels (e.g., lyophilized antibody panels) can be created, with each antibody panel being associated with a particular sample barcode by way of each antibody of that panel being coupled to a sample barcode sharing an identical unique isotope or identical combination of unique isotopes. In use, each sample-barcoded antibody panel can be separately combined with different samples, after which the samples can be washed and pooled together prior to interrogation using elemental analysis.

In some cases, a sample-specific antibody panel can include antibodies with element tags such that the combination of isotopes present in the sample after staining with the sample-specific antibody panel is unique to that sample. Put differently, a sample barcode can be incorporated into an antibody panel by distributing its unique combination of isotopes across multiple antibodies of the antibody panel.

In certain aspects, a live cell barcode (e.g., a thiol-reactive tellurium-based barcode, or an element tagged antibody to a widely expressed surface marker) could be used, which can add the benefit of also barcoding live cells in the sample (e.g., fresh blood). For example, a live cell barcode may include element tagged antibody that specifically binds CD45. This approach could be performed alongside a stimulation or another treatment of live cells (e.g., of PBMCs). In some cases, the sample barcode can be capable of barcoding live cells. In some cases, the sample barcode can be non-damaging to live cells, such as being non-toxic to live cells.

Certain aspects of the present disclosure can be useful for applications other than cytometry. For example, both an assay and sample barcode could be encoded in oligonucleotides attached to antibody bound beads, and a reporter antibody with an oligonucleotide could be used to detect analyte bound to beads. Beads can be isolated in droplets and a reaction product formed from the assay/sample barcode oligonucleotide on the beads and the oligonucleotide on the reporter antibody. This reaction product can be detected by sequencing, allowing for a high number of sample and assay barcodes to be detected.

Certain aspects of the present disclosure permit benefits unavailable to existing assays, such as deep immunophenotyping profiling for T cells; a broad coverage of additional immune lineages because of the ability to capture cell frequencies for other leukocytes (such as B cells, NK cells, dendritic cells and monocytes); maximized information content at the single cell level, especially with all of the markers combined and thus no need to infer between multiple tubes; the ability to control for technical variability with the use of sample barcodes; the ability to deeply dive into functions of interest by adding any panels or panel subsets needed for the function of interest; ability to provide a validated antibody cocktail that covers the needs of most consumers, thus reducing or eliminating wasted time and labor on validating slightly different cocktails; and ability to tailor data analysis to a particular purpose by achieving fast insight without operator bias.

In an example use case, many enterprises are racing to be first to market with the next product or label expansion. Their strategy is to execute early phase clinical trials in immune-oncology (I/O) as “basket trials” where a monotherapy or combination therapy is applied to multiple indications (e.g., cancer types) and patients are scrutinized for the faintest sign of clinical benefit. In these accelerated trial settings, each sample is particularly precious and it is crucial to maximize the information content of every analytical method, including flow cytometry. In addition, the adverse events for the classes of immunotherapies are immune-mediated, with potentially consequential information presenting in the comparison of baseline to on-treatment immuno-phenotype. These adverse events compound when used in combination, as is almost universally-accepted to be the future standard in the class, and where a large number of clinical trials have been initiated to test those combinations. Thus, aspects of the present disclosure can be especially useful for such enterprises.

In an example use case, contract research organizations act as the execution channel of choice for many enterprises' clinical trials, including most if not all flow cytometry tests. Aspects of the present disclosure can be especially useful for such research organizations, especially due to the cost and time efficiencies obtained, as well as the long-term stability of lyophilized panels as described herein.

In another example use case, large cancer research centers can serve as clinical trial sites for phase I/II, but also routinely treat patients with approved drugs. These centers may collect additional information to support research into development of new biomarkers and diagnostic tests. Aspects of the present disclosure can be especially useful for such research centers, especially due to the cost and time efficiencies obtained, as well as the long-term stability of lyophilized panels as described herein. In some cases, certain aspects of the present disclosure can be used as part of a lab-developed test that can be validated and used in their facility.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

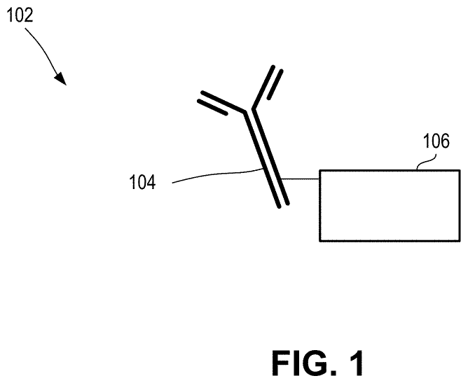

FIG. 1 is a schematic diagram of an element-tagged moiety 102 according to certain aspects of the present disclosure. The element-tagged moiety 102 of FIG. 1 is an antibody 104 that is tagged with an element tag 106, although any suitable moiety can be used when tagged with an element tag 106. The element tag 106 can be an isotope or combination of isotopes. In some cases, the element tag 106 can be a polymer chain having a plurality of metal containing pendant groups, such as a number of chelating groups. In some cases, multiple copies of a unique isotope can be contained within a single element tag 106. In some cases, multiple copies of the unique combination of isotopes can be contained within a single element tag 106, although that need not be the case.

The element-tagged moiety 102 can be part of a lyophilized antibody panel as disclosed herein. The antibody 104 can be any suitable antibody having a targeting function for a target of interest. By appropriately mixing the element-tagged moiety 102 with a sample, the antibody 104 can bind to any targets in the sample, thus labelling or tagging the sample with the element tag 106. Then, after washing to remove any unbound element tags, this stained sample can be interrogated using elemental analysis. Detection of the unique isotope or unique combination of isotopes by the elemental analyzer can be indicative of the presence of the element tag 106, and thus the element-tagged moiety 102, and thus any target to which the element-tagged moiety 102 is bound.

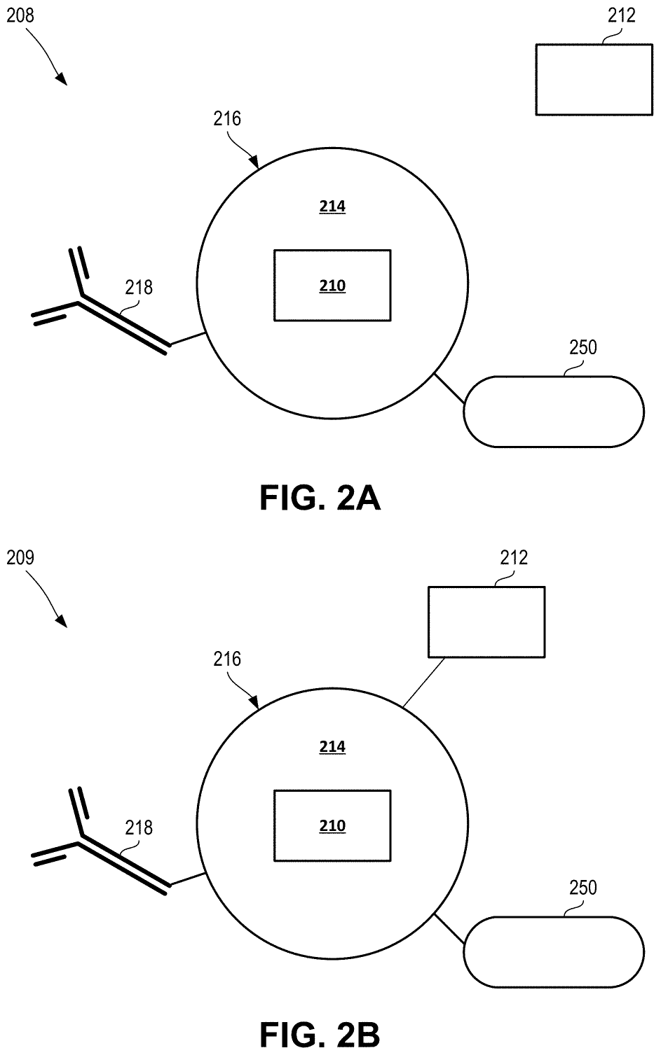

FIG. 2A is a schematic diagram of a barcoding reagent 208 with a separate sample barcode 212 according to certain aspects of the present disclosure. The barcoding reagent 208 can be a bead 214, although other barcoding reagents can be used. The bead 214 can contain an assay barcode 210 within the bead 214, such as incorporated into the core of the bead 214. The assay barcode 210 can be a type of element tag that includes a unique isotope or unique combination of isotopes distinguishable by elemental analysis. In some cases, the bead 214 can include a surface 216 that contains, binds, or is functionalized to bind various moieties, such as sample barcodes 212, capture antibodies 218, and/or assay-specific biomolecules 250.

The surface 216 of the bead 214 can bind or be functionalized to bind an affinity reagent for a particular assay, such as a particular capture antibody 218 or a particular assay-specific biomolecule 250, which can be a non-antibody biomolecule. The affinity reagent can bind to a particular target (e.g., protein or other structure), thus tagging the target with the assay barcode 210. Thus, after washing to remove any unbound barcoding reagent, detection of the assay barcode 210 is indicative of presence of the target to which the affinity reagent (e.g., capture antibody 218 or assay-specific biomolecule 250) is bound.

In some cases, the surface 216 of the bead 214 can be functionalized to bind a sample barcode 212. The sample barcode 212 can be an element tag containing a unique isotope or unique combination of isotopes discernable using elemental analysis. Multiple groups of sample barcodes can exist, with all sample barcodes from one group having identical isotopes or combinations of isotopes, and with samples barcodes from different groups being unique and differentiable from one another using elemental analysis. Thus, a set of any number of different barcoding reagents can be tagged with sample barcodes from the same group, thus associating each of those barcoding reagents with the sample associated with that group of sample barcodes. Separately, a different set of any number of different barcoding reagents can be tagged with sample barcodes from another group, thus associating that different set of barcoding reagents with the other group of sample barcodes.

Sample barcode 212 can be provided separately from the barcoding reagent 208, such as in a kit containing multiple different sample barcodes. Thus, when preparing for or conducting an assay using the barcoding reagent 208, the sample barcode 212 can be mixed with the barcoding reagent 208 before (e.g., by mixing the sample barcode 212 with the bead 214 before mixing both with the sample) or during the assay (e.g., by mixing the sample barcode 212 with the sample and then mixing the bead 214 therein).

FIG. 2B is a schematic diagram of the barcoding reagent 208 of FIG. 2A after attachment of the sample barcode 212 according to certain aspects of the present disclosure. In some cases, attachment of the sample barcode 212 can occur during manufacturing. In such cases, barcoding reagent 208 can be provided, such as part of a kit, with a sample barcode 212 bound to or otherwise contained within the surface 216 of the bead 214. In some cases, sample barcode 212 can be coupled to a barcoding reagent 208 prior to attachment of capture antibodies 218 and/or assay-specific biomolecules 250.

In some cases, attachment of the sample barcode 212 can be performed as part of preparing to conduct an assay (e.g., by mixing the sample barcode 212 with the bead 214 before mixing both with the sample) or as part of conducting the assay (e.g., by mixing the sample barcode 212 with the sample and then mixing the bead 214 therein).

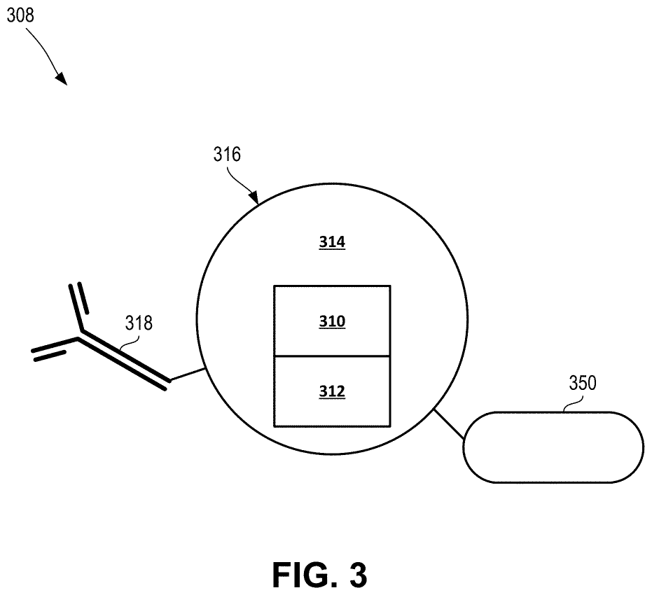

FIG. 3 is a schematic diagram of a barcoding reagent 308 with integrated sample barcode 312 according to certain aspects of the present disclosure. The barcoding reagent 308 can be a bead 314, although other barcoding reagents can be used. The bead 314 can contain both an assay barcode 310 and a sample barcode 312 within the bead 314, such as incorporated into the core of the bead 314. The assay barcode 310 and sample barcode 312 can each be types of element tags that include their own unique isotope or unique combination of isotopes discernable by elemental analysis.

In some cases, the bead 314 can include a surface 316 that contains, binds, or is functionalized to bind various attachments, such as capture antibodies 318 and assay-specific biomolecules 350. The surface 316 of the bead 314 can bind or be functionalized to bind an affinity reagent for a particular assay, such as a particular capture antibody 318 or a particular assay-specific biomolecule 350, which can be a non-antibody biomolecule. The affinity reagent can bind to a particular target (e.g., protein or other structure), thus tagging the target with the assay barcode 310. Thus, after washing to remove any unbound barcoding reagent, detection of the assay barcode 310 is indicative of presence of the target to which the affinity reagent (e.g., capture antibody 318 or assay-specific biomolecule 350) is bound.

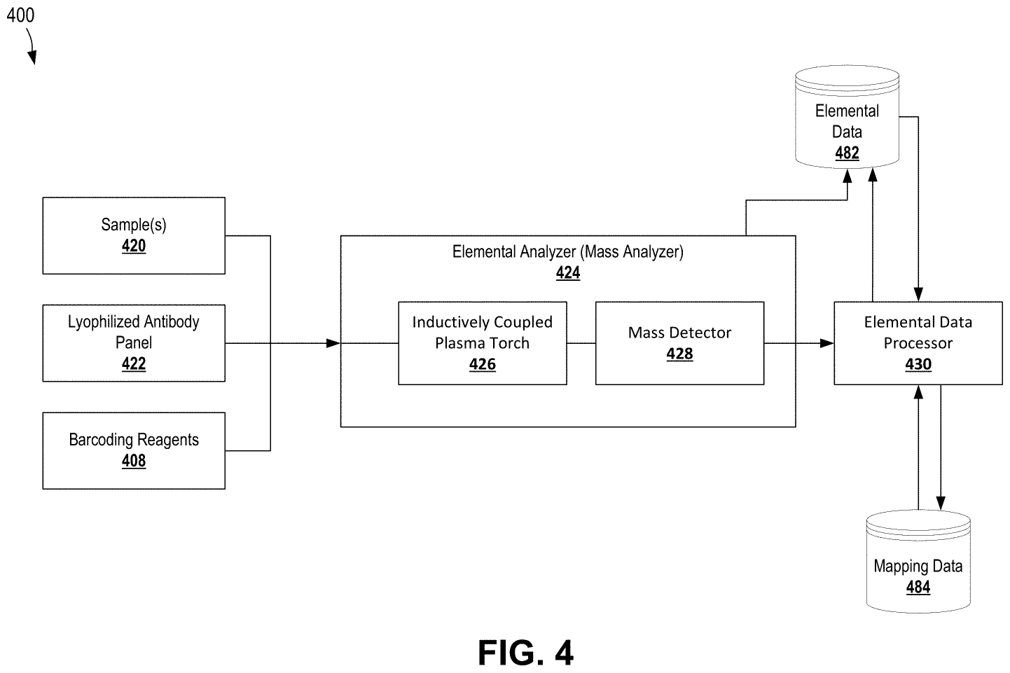

FIG. 4 is a schematic diagram of a system for assaying one or more samples 420 using an elemental analyzer 424 and a lyophilized antibody panel 422 according to certain aspects of the present disclosure. Sample(s) 420 can be any suitable sample(s) including whole blood or human peripheral blood, although any suitable sample can be used. Sample(s) 420 can be biological samples. Lyophilized antibody panel 422 can be a lyophilized antibody panel or panel subset as disclosed herein. The lyophilized antibody panel 422 can contain multiple element-tagged antibodies, such as element-tagged antibody 104 of FIG. 1.

The lyophilized antibody panel 422 can be mixed with the sample(s) 420 according to the appropriate protocol, resuspending the lyophilized antibody panel 422. In some cases, barcoding reagents 408 can be optionally mixed with the lyophilized antibody panel 422 and the sample(s) 420. Examples of suitable barcoding reagents 408 include sample barcoding reagents and assay barcoding reagents. In some cases, each of multiple samples 420 can be mixed with a respective set of barcoding reagents 408 in which each barcoding reagent of a set contains identical sample barcodes, thus applying unique sample barcodes to each of the multiple samples 420.

After being mixed with the lyophilized antibody panel 422, the sample(s) 420 can be interrogated using an elemental analyzer 424, such as a mass analyzer. In some cases, as depicted in FIG. 4, the elemental analyzer 424 can include an ICP-MS comprising an inductively coupled plasma torch 426 and a mass detector 428, although other elemental analyzers can be used. An inductively coupled plasma torch 426 can receive cells or particles of the stained and/or element-tagged sample(s) 420 and ionize the sample(s). The resultant stream of ions can be optionally processed (e.g., filtered) and passed to a mass detector 428. The mass detector 428 can be a time of flight detector or other suitable mass detector. The mass detector 428 can determine the presence and quantity of various isotopes in the ion stream based on the mass of the ions. The mass analyzer 428 can output data indicative of the presence and quantity of the various isotopes over time. The elemental analyzer 424 can thus output elemental data, which can include data indicative of the presence and quantity of the various isotopes over time. In some cases, elemental data can be stored in a datastore as elemental data 482, which can be later accessed for further analysis by an elemental data processor 430. In some cases, elemental data can be directed to an elemental data processor 430 for immediate analysis. In some cases, the elemental data processor 430 can be incorporated in the elemental analyzer, such as a software component of the elemental analyzer, although in some cases the elemental data processor 430 can be incorporated into a separate computing device.

The elemental analyzer 424 can obtain elemental data related to the presence and quantity of various isotopes over time. In some cases, this elemental data can be processed or segmented into time periods such that all elemental data within a particular time period (e.g., time window) can be associated with a single cell, bead, or particle detected by the elemental analyzer 424. For example, an elemental analyzer 424 analyzing a sample 420 containing a hundred cells may produce elemental data covering at least a hundred different time periods. Thus, the collection of all isotopes identified within a particular window can be used to resolve what targets or barcodes where detected in association with that particular cell, bead, or particle that is associated with that particular window.

In some cases, mapping data 384 can be stored in a datastore, which can be the same or a different datastore than the datastore where elemental data 482 is stored. The mapping data 484 can include information for associating an isotope or combination of isotopes to a particular target or object. The mapping data 484 can include information identifying expression of targets that distinguish cell types, thus facilitating automated cell type identification. For example, for element-tagged antibodies targeting CD20, CD45, CD14, CD16, CD8, CD3, and CD4, the mapping data 484 can identify respective isotopes ¹⁴⁷Sm, ¹⁵⁴Sm, ¹⁶⁰Gd, ¹⁶⁵Ho, ¹⁶⁸Er, ¹⁷⁰Er, and ¹⁷⁴Yb as being used to tag each of these element-tagged antibodies, thus mapping each isotope to a respective target. The mapping data 484 can be accessed by the elemental data processor 430.

In some cases, mapping data 484 can include mappings for any number of lyophilized antibody panels. The lyophilized antibody panel(s) being used (e.g., lyophilized antibody panel 422) can be manually (e.g., via user selection) or automatically (e.g., via direct sampling of the panel itself or via detection of a unique barcode within the stained sample(s)) detected, thus informing the elemental data processor 430 which mapping(s) to use. In some cases, the elemental data 482 can comprise additional metadata, such as an identification of which lyophilized antibody panel(s) are used. In addition to mappings of lyophilized antibody panel element tags to targets, the mapping data 484 can include mappings of other element tags to targets, such as mappings of sample barcodes to samples, mappings of assay barcodes to assays, and mappings of reporter barcodes to the reported target. The use of these additional mappings can be determined in the same fashion as how the mappings for a lyophilized antibody panel is determined.

Elemental data processor 430 can receive elemental data 482 and mapping data 484. The elemental data processor 430 can analyze the elemental data 482 using the mapping data 484 to obtain information about the sample(s), such as cytometric information about the cells or other particles within the sample(s). In some cases, elemental data processor 430 can separate the results by sample using different sample barcodes. In some cases, elemental data processor 430 can identify and quantify cell types based on the targets of the lyophilized antibody panel 422. In some cases, identifying and quantifying cell types can include applying a gating scheme to the elemental data, as disclosed in further detail herein.

The elemental data processor 430 can output results in any suitable fashion, such as those described herein. Example outputs can include storage to a datastore, presentation on a display; or input to further processes. The elemental data processor 430 can output any suitable types of information, such as those identified herein. For example, the elemental data processor 430 can output cell type results (e.g., relative quantification); marker intensities (e.g., a visual output showing the intensity “color” for each cell type in a phenotypic tree); marker frequencies, marker intensities, and cell counts; dot-plots of any two markers and/or histograms of any marker; or other suitable information.

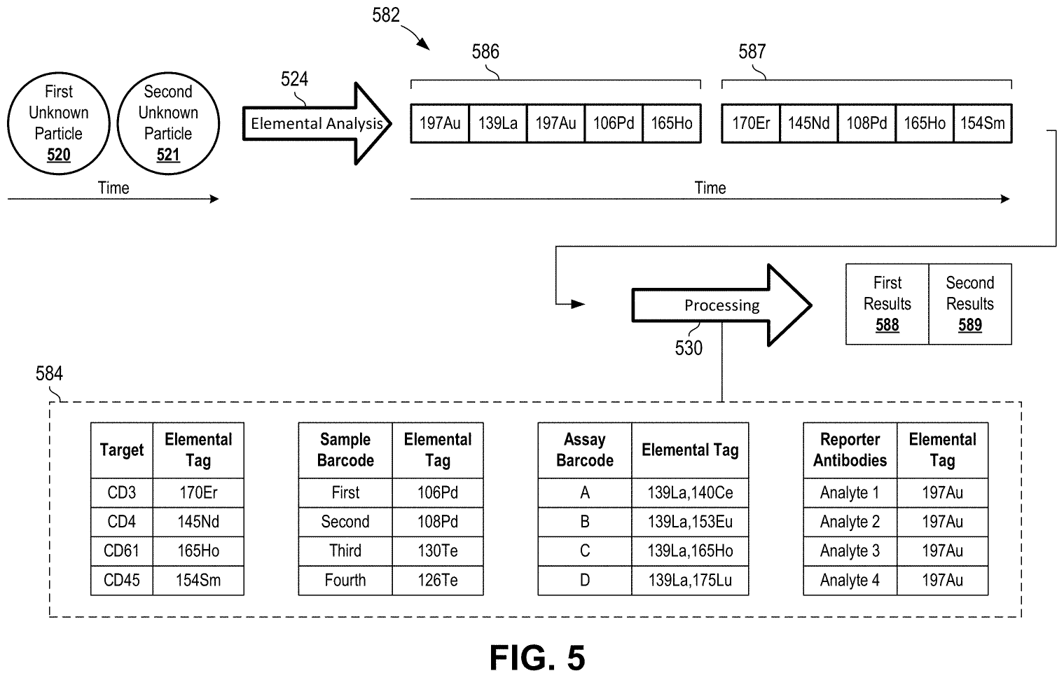

FIG. 5 is a schematic diagram depicting the analysis and processing of unknown particles 520, 521 according to certain aspects of the present disclosure. A first unknown particle 520 and second unknown particle 521 are subjected to elemental analysis 524. A time arrow is depicted to show that first unknown particle 520 is analyzed prior to second unknown particle 521. The first and second unknown particles 520, 521 can be particles of a sample, such as sample 420 of FIG. 4, after having been mixed with suitable antibody panels and/or reagents.

Elemental analysis 524 can result in elemental data 582 dividable into a first time window 586 and a second time window 587. The elemental data 582 can contain information associated with the isotopes detected by the elemental analyzer during elemental analysis 524. For example, the elemental data 582 can contain isotopes ¹⁹⁷Au, ¹³⁹La, ¹⁹⁷An, ¹⁰⁶Pd, and ¹⁶⁵Ho in the first time window 586 and isotopes ¹⁷⁰Er, ¹⁴⁵Nd, ¹⁰⁸Pd, ¹⁶⁵Ho, and ¹⁵⁴Sm in the second time window 587. The isotopes identified in the first time window 586 can be attributable to the ionization and detection of the first unknown particle 520. The isotopes identified in the second time window 587 can be attributable to the ionization and detection of the second unknown particle 521.

The elemental data 582 can be subjected to processing 530 to produce output results (e.g., first results 588 and second results 589. The processing 530, which can occur using an elemental data processor, such as elemental data processor 430 of FIG. 4, can make use of mapping data 584.

Mapping data 584 can contain information for identifying targets or barcodes based on detected element tags. As depicted in FIG. 5, certain example isotopes are used in the given element tags for illustrative purposes, although other element tags and other isotopes can be used. In an example, cell type mapping data can include a number of targets (e.g., CD3, CD4, CD61, CD45) and their respective associated element tags (e.g., ¹⁷⁰Er, ¹⁴⁵Nd, ¹⁶⁵Ho, ¹⁵⁴Sm); sample barcoding data can include a number of samples (e.g., First, Second, Third, Fourth) and their respective associated element tags (e.g., ¹⁰⁶Pd, ¹⁰⁸Pd, ¹³⁰Te, ¹²⁶Te), assay barcoding data can include a number of assays (e.g., A, B, C, D) and their respective associated element tags (e.g., ¹³⁹La and ¹⁴⁰Ce, 139La and ¹⁵³Eu, ¹³⁹La and ¹⁶⁵Ho, ¹³⁹La and ¹⁷⁵Lu); and reporter antibody data can include a number of targets (e.g., Analyte 1, Analyte 2, Analyte 3, and Analyte 4) and their common associated element tag (e.g., ¹⁹⁷Au). In certain aspects, the reporter antibody may include a high sensitivity element tag (e.g., a gold nanoparticle) and a lower sensitivity element tag (e.g., a polymer chelated to lanthanide atom(s)).

The isotopes from the first time window 586 include multiple instances of detected isotope ¹⁹⁷Au, so an inference can be made that the first unknown particle 520 is a target analyte (e.g., one of Analyte 1, Analyte 2, Analyte 3, Analyte 4). This reporter reagent can be used to target only target analytes which assay beads would target. Thus, an inference can be made that the first unknown particle 520 is not a cell, but is rather an assay bead (e.g., an assay bead coupled to a target analyte). The other detected isotopes in that first time window 586 include ¹³⁹La, and ¹⁶⁵Ho, indicating the first unknown particle 520 is likely assay bead C. Further, the detected isotopes in the first time window 586 include ¹⁰⁶Pd, indicating that the first unknown particle 520 is part of a First sample. This information can be provided as part of first results 588.

The isotopes from the second time window 587 do not include any isotopes associated with reporter antibodies associated with targets of assay beads. Rather, the isotopes from the second time window 587 include ¹⁷⁰Er, ¹⁴⁵Nd, ¹⁶⁵Ho, and ¹⁵⁴Sm, indicating the second unknown particle 521 contains CD3, CD4, CD61, and CD45, respectively. Further, an inference can be made that the second unknown particle 521 is a cell. With sufficient further cell type target information, an inference can be made as to the type of cell, as disclosed in further detail herein. While both the first time window 586 and second time window 587 contain ¹⁶⁵Ho, the lack of a reporter antibody, and optionally the presence of other cell type targets (e.g., CD3, Cd4, CD45), is indicative that the detected ¹⁶⁵Ho isotope is from the cell type antibody, and not from an assay barcode. Finally, the presence of the ¹⁰⁸Pd isotope in the second time window 587 is indicative that the second unknown particle 521 is part of a Second sample. This information can be provided as part of second results 589.

Thus, despite the first and second unknown particles 520, 521 being analyzed by the elemental analyzer sequentially, processing of the elemental data can still differentiate the particles 520, 521 into their respective particle types and into their respective samples.

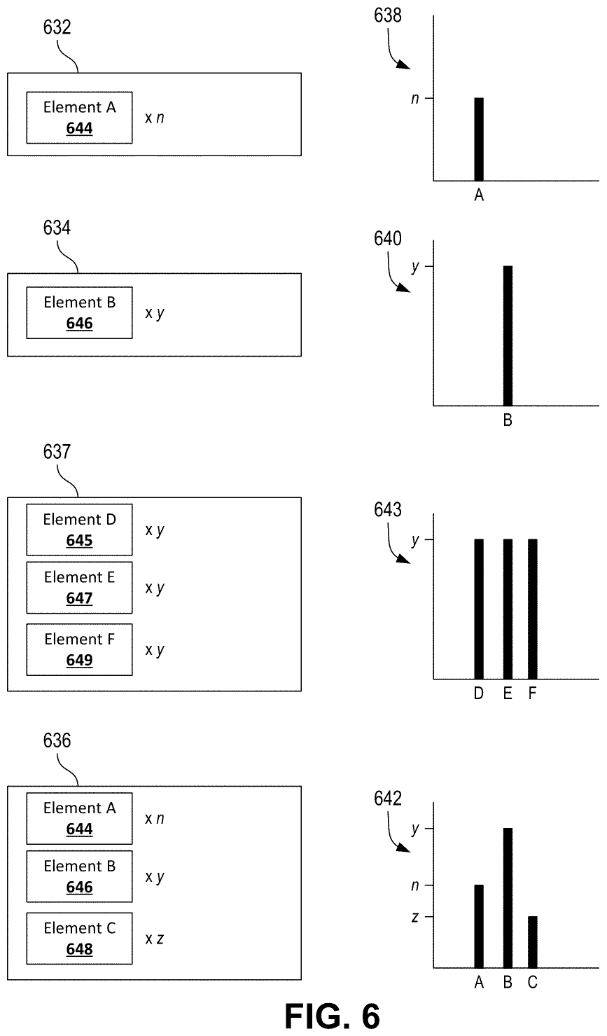

FIG. 6 is a schematic diagram depicting three example element tags 632, 634, 636 with charts 638, 640, 642 illustrating their respective elemental data according to certain aspects of the present disclosure. Each element tag 632, 634, 636 can be an element tag as used in an element-tagged moiety (e.g., element-tagged moiety 102 of FIG. 1), a sample barcode (e.g., sample barcode 212 of FIG. 2), or an assay barcode (e.g., assay barcode 210 of FIG. 2). The charts 638, 640, 642 associated with respective element tags 632, 634, 636 illustrate the actual or relative strength of expression of the particular element as detected by an elemental analyzer (e.g., elemental analyzer 424 of FIG. 4) interrogating a sample containing the respective element tag 632, 634, 636. As used herein, the terms “Element A,” “Element B,” or “Element C” refer to generic elements that can be used for tagging element tags, such as metals.

Element tag 632 is an example of a single-isotope element tag. Element tag 632 consists of Element A 644, and no other elements used to differentiate this element tag from other element tags (e.g., the element tag 632 may include other elements, such as carbon and hydrogen, but these other elements are structural and not for differentiation purposes). This element tag 632 can comprise one or more instances of Element A 644, such as n different copies of the isotope. Upon interrogation by an elemental analyzer, the elemental data for element tag 632 can show a relative expression for element A at a level corresponding to the number n of copies of Element A 644 in the element tag 632.

Element tag 634 is an example of a single-isotope element tag that is differentiable from element tag 632. Element tag 634 consists of Element B 646. This element tag 634 can comprise one or more instances of Element B 646, such as y different copies of the isotope. Upon interrogation by an elemental analyzer, the elemental data for element tag 634 can show a relative expression for element B at a level corresponding to the number y of copies of Element B 646 in the element tag 634. Thus, element tag 634 is isotopically distinguishable from element tag 634 because a different isotope is detected, and optionally because a different isotope is detected at a different relative expression.

Element tag 637 is an example of a multi-isotopes element tag that is distinguishable from element tags 632, 634. Element tag 637 consists of Element D 645, Element E 647, and Element F 649. This element tag 637 can comprise one or more instances each of Element D 645, Element E 647, and Element F 649. As depicted in FIG. 6, element tag 637 contains y different copies of Element D 645, Element E 647, and Element F 649. Upon interrogation by an elemental analyzer, the elemental data for element tag 637 can show the presence of (e.g., and optionally a relative expression for) elements D, E, and F in the element tag 637. Thus, element tag 637 is isotopically distinguishable from element tags 634, 636 because a unique combination of isotopes is detected. The unique combination can be identified based on a list of the isotopes detected (e.g., elements D, E, and F), optionally based on a relative expressions between the detected isotopes in the element tag, and/or optionally based on actual expression levels of the detected isotopes when compared to other element tags.

Element tag 636 is an example of a multi-isotope element tag that is differentiable from element tags 632, 634, 637. Element tag 636 consists of Element A 644, Element B 646, and Element C 648. This element tag 636 can comprise one or more instances of Element A 644, Element B 646, and Element C 648, such as n different copies of Element A 644, y different copies of Element B 646, and z different copies of Element C 648. Upon interrogation by an elemental analyzer, the elemental data for element tag 636 can show a relative expression for elements A, B, and C, at levels corresponding to the n, y, and z copies of the respective elements in the element tag 636. Thus, element tag 636 is isotopically distinguishable from element tags 634, 636, 637 because a unique combination of isotopes is detected. The unique combination can be identified based on a list of the isotopes detected (e.g., elements A, B, and C), optionally based on a relative expressions between the detected isotopes in the element tag (e.g., the expression of element C is less than the expression of element A, which is less than the expression of element B), and/or optionally based on actual expression levels of the detected isotopes when compared to other element tags (e.g., A:n, B:y, C:n may be associated with element tag 636, whereas another element tag may be associated with A:z, B:n, C:y).

Numerous unique element tags can thus be created with unique individual isotopes or unique combinations of isotopes. In some cases, a combination of isotopes can refer to a combination comprising a single isotope at a particular expression level, which can be distinguishable from a different combination of isotopes comprising the same single isotope at a different and distinguishable expression level.

Each unique isotope or combination of isotopes can be considered to be an elemental barcode, which can be used to identify any moiety to which it is coupled. Thus, when coupled to a moiety having a targeting function for a particular target, the elemental barcode can be used to identify the target after the element-tagged moiety has been contacted with a sample containing the target and non-bound element-tagged moieties have been washed away.

Depending on the type of elemental analysis, a certain number of isotopes can be reliably distinguishable. For example, an example ICP-MS system may be able to discern up to x different mass channels, thus being able to reliably discern x different isotopes (e.g., isotopes 1, 2, 3, 4, 5, . . . , x). Thus, unique barcodes can be generated for at least 2^{x} different combinations of isotopes based solely on determining the presence of the detectable isotope in the element tag. Out of these 2^{x} different combinations, it can be desirable to remove certain combinations of isotopes likely to be detected in a sample. For example, given a set of isotopes or combination of isotopes used as element tags in a lyophilized antibody panel or used across a set of potential lyophilized antibody panels, it may be desirable to use entirely different isotopes for sample barcoding and/or assay barcoding, so as to avoid the possibility of combinations of antibodies detected simultaneously or near-simultaneously as being misconstrued as being a different combination of isotopes. For example, element tag 636 can include Element C 648 so that even if element tags 632, 634 were detected simultaneously or near-simultaneously, the analyzer or processor would not erroneously construe the detection of elements A and B as being element tag 636, since no element C was detected. In some cases, element tag 636 can be further protected from the possibility of erroneous identification by not using any of the isotopes from element tags 632, 634.

In some cases assay barcodes and/or sample barcodes are configured to have non-overlapping isotopes, although that need not always be the case. In some cases, the assay barcodes and/or sample barcodes can include unique combinations of a number of isotopes out of a set of possible isotopes. For example, if a total of six different isotopes are being used for sample barcoding, each sample barcode can include a unique combination of three of the six total isotopes, thus resulting in twenty different unique sample barcodes. For example, where the total number of possible unique isotopes used for a barcode is n and the number of unique isotopes chosen for each barcode is k, the total number of possible unique barcodes is (n!/k!(n−k)!).

In some cases element tags used for cell type identification contain one or more of a single isotope, such as element tags 632, 634, since often assays will involve detecting numerous tags all coupled to a single cell. In some cases, element tags used for barcoding assay beads contain unique combinations of isotopes, since the likelihood of detecting multiple assay beads within a single time window is minimal, and thus the chance of detecting overlapping element tags is minimal. The use of unique combinations of isotopes for assay beads permits more unique element tags to be achieved with a smaller number of unique isotopes.

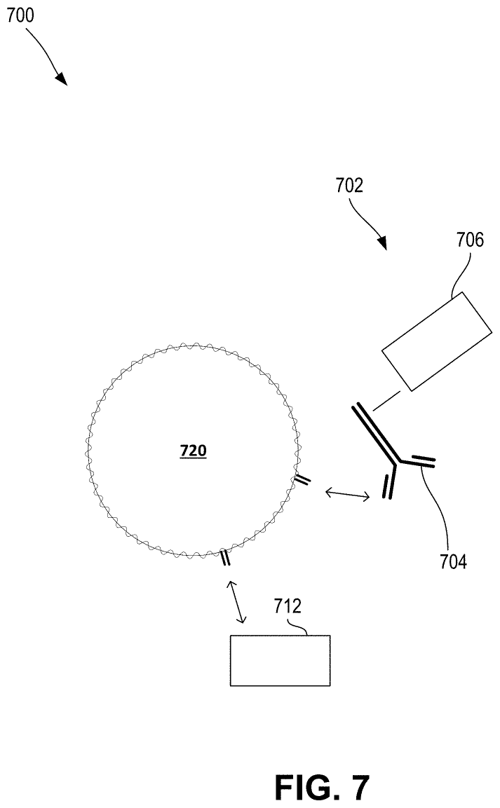

FIG. 7 is a schematic diagram of a sample cell 720 tagged with an element-tagged moiety 702, a sample barcode 712, and a barcoding reagent 708 according to certain aspects of the present disclosure. The sample cell 720 can be any cell of a sample, such as a blood cell. In some cases, instead of a sample cell 720, another particle or target can be used, such as proteins found in blood plasma (e.g., albumin). The sample cell 720 of FIG. 7 is depicted as being tagged by an element-tagged moiety 702, a sample barcode 712, and a barcoding reagent 708, although in various uses described herein, such a sample may be tagged with any combination of one, two, all three, or more of these objects.

Element-tagged moiety 702 can be any suitable moiety (e.g., element-tagged moiety 102 of FIG. 1), such as an element-tagged antibody of a lyophilized antibody panel. The antibody 704 can be bound to an element tag 706. The antibody 704 can bind to the sample cell 720 through any suitable means, such binding to an antigen on the surface of the sample cell 720. In some cases, an element-tagged moiety can infiltrate a sample cell 720 (e.g., a permeabilized sample cell 720) and bind to an intracellular target.

The element tag 706 associated with the element-tagged moiety 702 can be the same across all element-tagged moieties 702 with the same antibody 704, but element-tagged moieties with a different antibody (e.g., targeting a different target) can have a unique element tag.

Sample barcode 712 can be an element tag that binds to the sample cell 720 directly or via an intermediary moiety (e.g., an antibody). The sample barcode 712 can be designed to bind to all, a supermajority, or a majority of cells or particles of a sample. Each sample barcode 712 can have the same isotope or combination of isotopes across all of the sample barcodes 712.

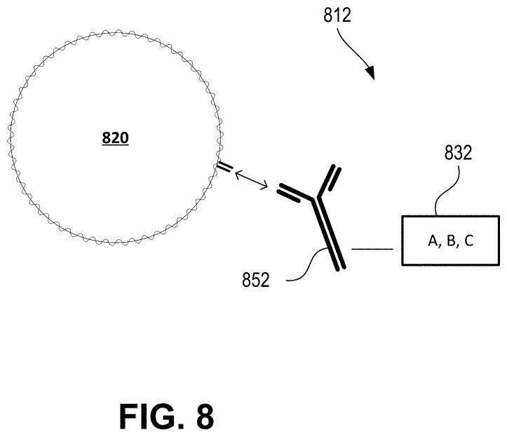

FIG. 8 is a schematic diagram depicting a sample cell 820 tagged with a multi-isotope sample barcode 812 according to certain aspects of the present disclosure. Sample cell 820 can be sample cell 720 of FIG. 7. A sample barcode 812 can be coupled to the sample cell 820. The sample barcode 812 can comprise an antibody 852 that having a targeting function for the sample cell 820 (e.g., having a targeting function for a cell surface target of an intracellular target). The antibody 852 of the sample barcode 812 can be coupled to an element tag 832 comprising the unique combination of isotopes associated with the sample barcode 812. For example, the sample barcode 812 can have a hypothetical barcode of “A, B, C,” where A, B, and C, refer to unique isotopes (e.g., ¹⁰²Pd, ¹⁰⁴Pd, ¹⁰⁵Pd).

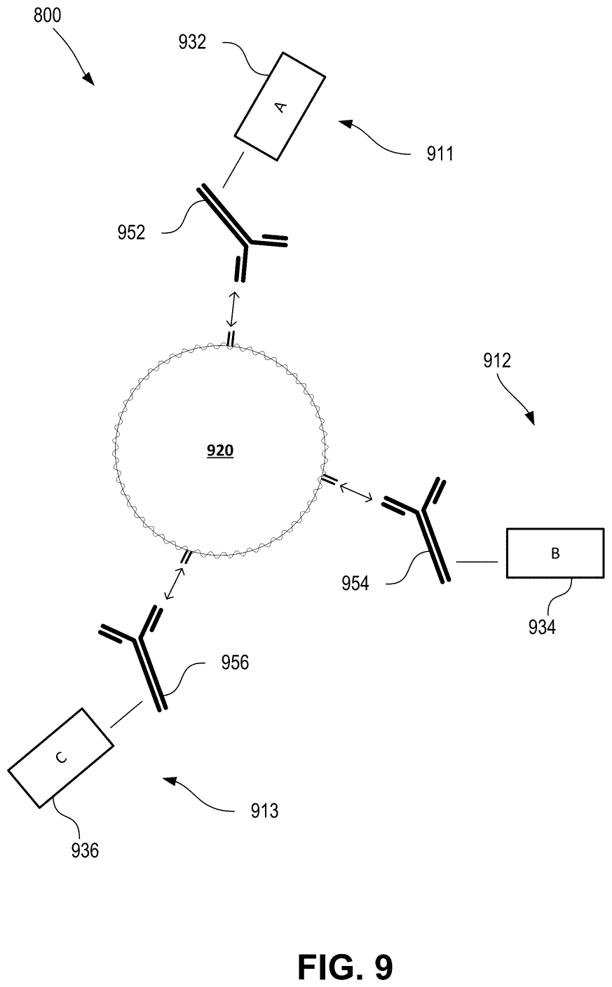

FIG. 9 is a schematic diagram depicting a sample cell 920 tagged with distributed sample barcode parts 911, 912, 913 according to certain aspects of the present disclosure. Sample cell 920 can be sample cell 720 of FIG. 7. The distributed sample barcode parts 911, 912, 913 of sample cell 920 can result in the same overall barcode being applied to sample cell 920 as sample barcode 812 applied to sample cell 820 of FIG. 8.

Sample cell 920 can include multiple distributed sample barcode parts 911, 912, 913 coupled thereto. Each sample barcode part 911, 912, 913 can include respective antibodies 952, 954, 956, each coupled to respective element tags 932, 934, 936. Each antibody 952, 954, 956 in a distributed sample barcode system can be the same or can be different as long as the antibodies all have targeting functions associated with the same sample cell 920. For example, antibody 952 can target CD45, antibody 954 can target CD298, and antibody 956 can target b2m. The isotopes of the overall sample barcode (e.g., hypothetical isotopes “A, B, and C”) can be distributed across the various barcode parts 911, 912, 913. Thus, element tag 932 can contain isotope A, element tag 934 can contain isotope B, and element tag 936 can contain isotope C. Thus, when the sample cell 920 is interrogated using an elemental analyzer, the same overall sample barcode of “A, B, C” will be detected, despite the overall sample barcode being distributed across multiple barcode parts 911, 912, 913.

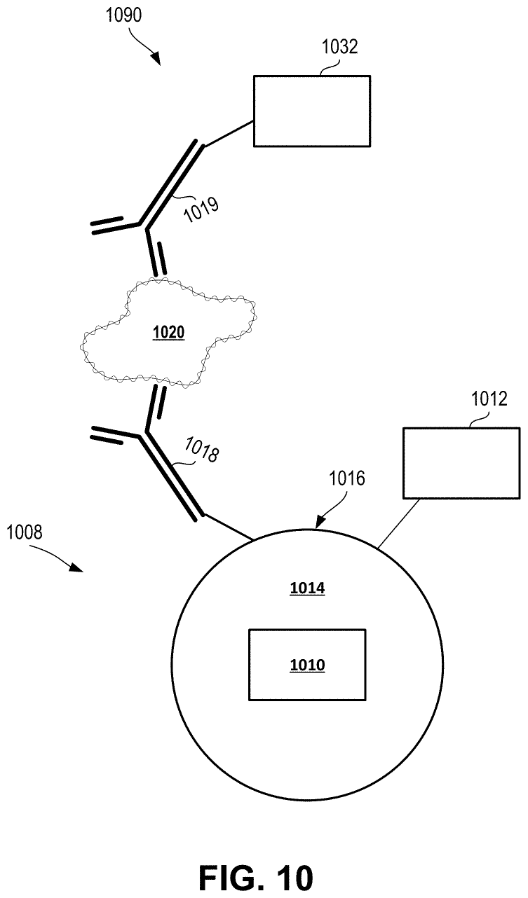

FIG. 10 is a schematic diagram of a barcoding reagent 1008 and reporter antibody 1019 according to certain aspects of the present disclosure. The barcoding reagent 1008 can be a bead 1014, although other barcoding reagents can be used. The bead 1014 can contain an assay barcode 1010 within the bead 1014, such as incorporated into the core of the bead 1014. The assay barcode 1010 can be a type of element tag that includes a unique isotope or unique combination of isotopes discernable by elemental analysis. In some cases, the bead 1014 can include a surface 1016 that contains, binds, or is functionalized to bind various attachments, such as sample barcodes 1012, capture antibodies 1018, and assay-specific biomolecules.

The surface 1016 of the bead 1014 can bind or be functionalized to bind an affinity reagent for a particular assay, such as a particular capture antibody 1018 or a particular assay-specific biomolecule, which can be a non-antibody biomolecule. The affinity reagent can bind to a particular target analyte 1020 (e.g., protein or other structure), thus tagging the target analyte 1020 with the assay barcode 1010. Thus, after washing to remove any unbound barcoding reagent, detection of the assay barcode 1010 is indicative of presence of the target analyte 1020 to which the affinity reagent (e.g., capture antibody 1018 or assay-specific biomolecule 1050) is bound.

In some cases, the surface 1016 of the bead 1014 can bind, contain, or be functionalized to bind a sample barcode 1012. The sample barcode 1012 can be an element tag containing a unique isotope or unique combination of isotopes discernable using elemental analysis. The particular sample barcode 1012 bound to bead 1014 can be specific to the sample from which the target analyte 1020 originated. By using multiple groups of sample barcodes with unique isotopes or combinations of isotopes, each original sample can be assayed using its own group of sample barcodes (e.g., mixed with the sample or coupled to assay reagent 1008 that is then mixed with the sample), and thus detection of the particular sample barcode 1012 can be used to identify from which sample the target analyte 1020 originated.

In some cases, a reporter reagent 1090 can be used. The reporter reagent 1090 can include a reporter antibody 1019 that has a targeting function for the target analyte 1020. The reporter antibody 1019 can be coupled to an element tag 1032 containing an isotope or unique combination of isotopes. Detection of the isotope or unique combination of isotopes associated with element tag 1032 can be indicative that the particle detected by the elemental analyzer, and thus the other isotopes detected within a particular time window, are associated with the target analyte 1020, and thus associated with the assay bead 1014.

Multiple different reporter reagents 1090 can be used, each having different types of reporter antibodies with targeting functions for various different target analytes, although all reporter reagents 1090 can include the same type of element tag (e.g., the same isotope or combination of isotopes). Thus, each reporter reagent 1090 would have an identical element tag 1032. Since the purpose of a reporter reagent 1090 is only to determine if a particular object (e.g., assay bead 1014) is being interrogated by the elemental analyzer, there is no need to distinguish between different reporter reagents 1090.

In some cases, the reporter antibody 1019 can be the same type of antibody (e.g., monoclonal antibodies) as the capture antibody 1018 of the assay bead 1014. In some cases, the reporter antibody 1019 is a different type of antibody (e.g., polyclonal antibody) from the capture antibody 1018 of the assay bead 1014, although both the reporter antibody 1019 and the capture antibody 1018 have targeting functions for the target analyte 1020.

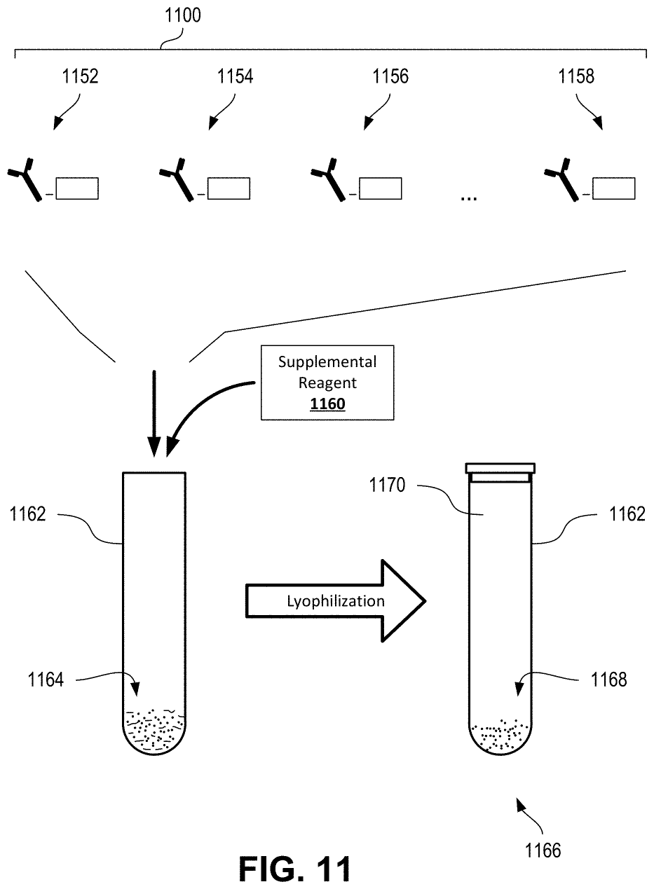

FIG. 11 is a schematic diagram depicting the preparation of a lyophilized antibody panel 1166 according to certain aspects of the present disclosure. The lyophilized antibody panel 1168 can first start as a set 1100 of element-tagged antibody groups 1152, 1154, 1156, 1158. The set 1100 can include any number of antibody groups. Each antibody group can include one or more element-tagged antibodies that share identical antibodies and identical element tags. For a panel targeting six different targets, the set 1100 can include six different antibody groups, with each group containing multiple individual element-tagged antibodies.

The set 1100 of element-tagged antibody groups 1152, 1154, 1156, 1158 can be prepared as necessary, such as described herein. As an example, each of the element-tagged antibody groups 1152, 1154, 1156, 1158 can be individually titrated and diluted in stabilizers to achieve desired concentrations. The resultant solutions can be individually combined with excipients and mixed together to form an admixture 1164, which can be placed in a container 1162, such as a tube. In some cases, supplemental reagents 1160 can be added to the admixture 1164. Such supplemental reagents 1160 can include any reagents capable of being lyophilized. For example, lysing reagents (e.g., red blood cell lysing reagents), wash buffers, fixation reagents, and/or permeabilizing reagents can be added as a supplemental reagent 1160. In some cases, a barcoding reagent, such as an assay barcoding reagent or a sample barcoding reagent (e.g., a sample barcodes) can be added to the admixture 1164 as a supplemental reagent 1160. In some cases, one or more calibration materials (e.g., calibration beads) can be added as a supplemental reagent 1160 or supplemental material.

The admixture 1164 can be subjected to a lyophilization process, such as disclosed herein. For example, the admixture 1164 can be subjected to a thermal stage, an evacuation stage, a drying stage, and a holding stage. During a thermal stage, the temperature can be held somewhere between −60 and 0° C. for a duration while the vacuum is held in a range between 100-500 Torr. During an evacuation stage, the vacuum can be brought down to a range between 100-500 mTorr. During a drying stage, the temperature can be manipulated between ranges of −50 to 30° C. while the vacuum is held in a range of 10-150 mTorr. During a holding stage, the temperature can be maintained at a temperature between 10 and 30° C., while the vacuum is permitted to raise to a range between 100-500 mTorr. After the holding stage, the container 1162 can be backfilled, which can include raising the pressure in the container, such as up to a range from 200 Torr up to 760 Torr, although in some cases, backfilling occurs at a pressure below ambient pressure. In some cases, during all stages of lyophilization, the temperature of the admixture 1162 does not rise above a glass transition temperature of the admixture 1162. During or after backfilling, the container 1162 can be filled with an internal atmosphere 1170 of inert gas or dry air, then sealed.

After lyophilization, the container 1162 can contain a lyophilized admixture 1168 making up the lyophilized panel 1166. This lyophilized panel 1166 can be stored and later resuspended for staining a sample.

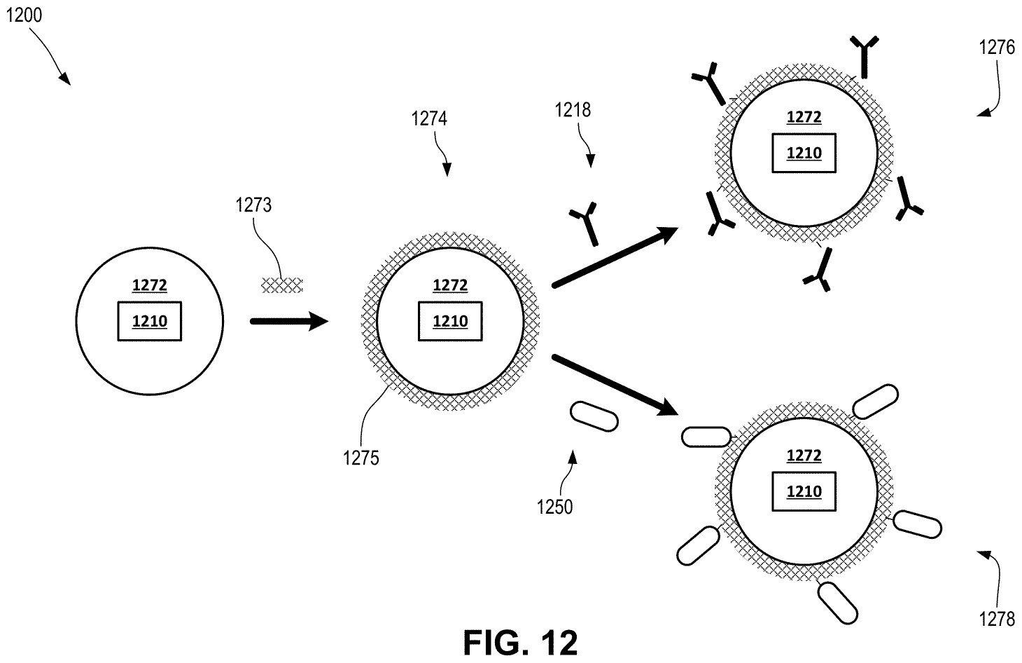

FIG. 12 is a schematic diagram of a process 1200 for preparing barcoding reagents 1276, 1278 according to certain aspects of the present disclosure. The process 1200 can start with a element-tagged core 1272. The element-tagged core 1272 can include any suitable element tag 1210, such as an element tag 1210 containing a combination of isotopes selected from La, Ce, Eu, Ho, and Lu. The element-tagged core 1272 can include the isotopes as part of a solid metal core, or a polymer core that chelates or otherwise entraps metal isotopes.

Polymer precursors 1273 (e.g., subunits) can be reacted with the element-tagged core 1272 to generate an element-tagged bead 1274 comprising an element-tagged core 1272 surrounded by a polymer shell 1275. This element-tagged bead 1274 can be reacted with either capture antibodies 1218 or assay-specific biomolecules 1250.

In some cases, the element-tagged bead 1274 with a polymer shell 1275 can be reacted with capture antibodies 1218 having an exposed amine group (e.g., on the Fc region), permitting the capture antibodies 1218 to form covalent bonds with the polymer shell 1275, thus resulting in an antibody-based element-tagged barcoding reagent 1276.

In some cases, the element-tagged bead 1274 with a polymer shell 1275 can be reacted with assay-specific biomolecules 1220 having an exposed amine group, permitting the assay-specific biomolecules 1220 to form covalent bonds with the polymer shell 1275, thus resulting in a biomolecule-based element-tagged barcoding reagent 1278.

In some cases, the antibody-based element-tagged barcoding reagent 1276 and/or biomolecule-based element-tagged barcoding reagent 1278 can be further tagged with a sample barcode such as using a sample barcode comprising a highly reactive functional group capable of binding to the polymer shell 1275, displacing an antibody 1218 or biomolecule 1250 from the polymer shell 1275, or binding to an additional functional group present on the barcoding reagent 1276, 1278.

FIG. 13 is a flowchart depicting a process 1300 for staining and analyzing a blood sample according to certain aspects of the present disclosure. While process 1300 is disclosed with reference to analyzing a sample of whole blood, aspects of process 1300 can be used to analyze other samples, as appropriate.

At block 1302, a sample of whole blood is provided. The whole blood can be human peripheral blood, whether fresh or frozen. In some cases, at optional block 1304, the whole blood can be tagged with a sample barcode 1304.

At block 1306, PBMCs from the sample of whole blood can be isolated. The PBMCs can be isolated using any suitable technique, such as via centrifugation. In some cases, the PBMCs can be optionally tagged with a sample barcode at block 1308.

At block 1310, the PBMCs can be stained using a lyophilized panel, such as a lyophilized antibody panel or panel subset disclosed herein. In some cases, staining the PBMCs with the lyophilized panel can include recording information for the type of lyophilized panel used, which information can be retrieved to determine the panel's mapping between element tags and targets. Staining the PBMCs can include mixing the lyophilized panel with the PBMCs for a duration and at a temperature, then washing away unbound antibodies from the PBMCs. In some cases, the stained sample can be optionally tagged with a sample barcode at block 1312.

At optional block 1313, further cell processing can occur. Further cell processing can include additional processing and/or staining steps, such as cell fixation, cell permeabilization, and/or intracellular staining. Further cell processing at block 1313 can occur after sample barcoding at block 1312, although that need not always be the case.

At block 1314, plasma can be isolated from the sample of whole blood. The plasma can be isolated using any suitable technique, such as via centrifugation. In some cases, the plasma can be optionally tagged with a sample barcode at block 1316.

At block 1318, free analyte bead assays with reporter antibodies are added to the plasma. The free analyte bead assays can comprise one or more barcoding reagents with assay barcodes and capture antibodies or assay-specific biomolecules for targeting the free analytes of the plasma. Thus, captured antibodies or assay-specific biomolecules that bind to free analytes can tag those free analytes with their respective assay barcodes. The use of a reporter antibody can be optional. The reporter antibody can be used to identify the presence of a bead or an analyte. The reporter antibody can be selected to bind to all, a supermajority of, or a majority of beads or analytes. All reporter antibodies for a particular object can share the same element tag, such that detection of that element tag is indicative of the presence of that type of object (e.g., a bead or analyte). Adding the free analyte bead assays at block 1318 can include mixing the plasma with the free analyte bead assays for a duration at a temperature, then washing away unbound beads from the plasma. In some cases, the stained plasma can be optionally tagged with a sample barcode at block 1320.

At optional block 1322, the PBMC samples and plasma samples can be pooled together. In some cases, if sample barcoding is applied at blocks 1304, 1308, 1312, 1316, and/or 1320, the stained samples (e.g., PBMCs and/or plasma) derived from the whole blood of block 1302 can be pooled together with stained samples from other sources of whole blood (e.g., a whole blood from a different patient or whole blood from the same patient taken at different time than the whole blood from block 1302). Due to the sample barcoding, interrogation of the pooled samples can result in data containing unique sample barcodes that can be used to separate the data by the original sample source.

At block 1324, the stained sample(s) (e.g., PBMCs and/or plasma), with or without being optionally pooled at block 1322, can be interrogated with an elemental analyzer, such as a mass analyzer (e.g., an ICP-MS). The interrogation at block 1324 can result in elemental data for the sample(s).

At block 1326, the elemental data for the sample(s) can be automatically analyzed. Automatic analysis at block 1326 can include accessing a mapping of element tags to targets. Automatic analysis at block 1326 can include identifying cells or particles of the sample(s). Automatic analysis at block 1326 can include associating identified cells or particles to a particular sample based on a detected elemental tag associated with a sample barcode. Automatic analysis at block 1326 can include associating identified cells or particles to a particular assay based on a detected assay barcode. Automatic analysis at block 1326 can include associating identified cells or particles to one or more particular markers based on detecting element tags associated with the one or more particular markers. In some cases, automatic analysis at block 1326 can include identifying a type of a cell or particle based on associated markers. In some cases, automatic analysis at block 1326 can include generating output containing information associated with quantification or identification of cells or cell types in the sample. The automatic analysis at block 1326 can include generating any suitable output from elemental data as disclosed herein.

Certain methods and kits may only include a subset of the blocks described in FIG. 13. For example, sample barcoding may not be performed, although cells and assay beads from the same sample may still be combined before interrogation. Alternatively, sample barcoding of cells and/or assay beads (e.g., separately or in mixture) may be performed at any step prior to interrogation, and pooling of cells and/or assay beads from different samples may be performed at any point after sample barcoding and before interrogation. For example, a sample barcode may be introduced at any step, and pooling of samples performed in any later step. PBMCs in serum and plasma may not be separated, and may instead be applied to a lyophilized mixture of element-tagged antibodies and assay barcoded beads, allowing for a more streamlined workflow (although the quality of cell staining may be reduced due to an abundance of free analytes). A sample barcode may be included in mixture with the lyophilized mixture, or provided separately (for addition to the sample before or after staining with the lyophilized panel). In certain aspects, a tissue sample or cell culture may be provided in place of whole blood in step 1302 and cell isolation in step 1306 is optional. In certain aspects, step 1318 is the addition of a reporter reagent comprising a biomolecule other than an antibody. In an example where cells and not assay beads are interrogated, a method may include (in order) steps 1302, 1308, 1310, optionally 1313, 1324, and 1326. In an example where assay beads and not cells are interrogated, a method may include (in order) steps 1302, 1314, 1316, 1322, and 1324. In some examples, the reporter antibody may be added separate from (before or after) the bead assay is added, such as after addition of the bead assay with a wash step in-between. In some examples, cells are permeabilized before block 1310 and the lyophilized panel stain comprises antibodies to both extracellular and intracellular targets. In certain examples, cells (e.g., PBMCs) and free analytes (e.g., plasma) are isolated prior to addition of the lyophilized panel and bead assay respectively, and combined prior to sample barcoding and pooling across samples. In some cases, cells are sample barcoded and pooled prior to addition to the lyophilized panel.

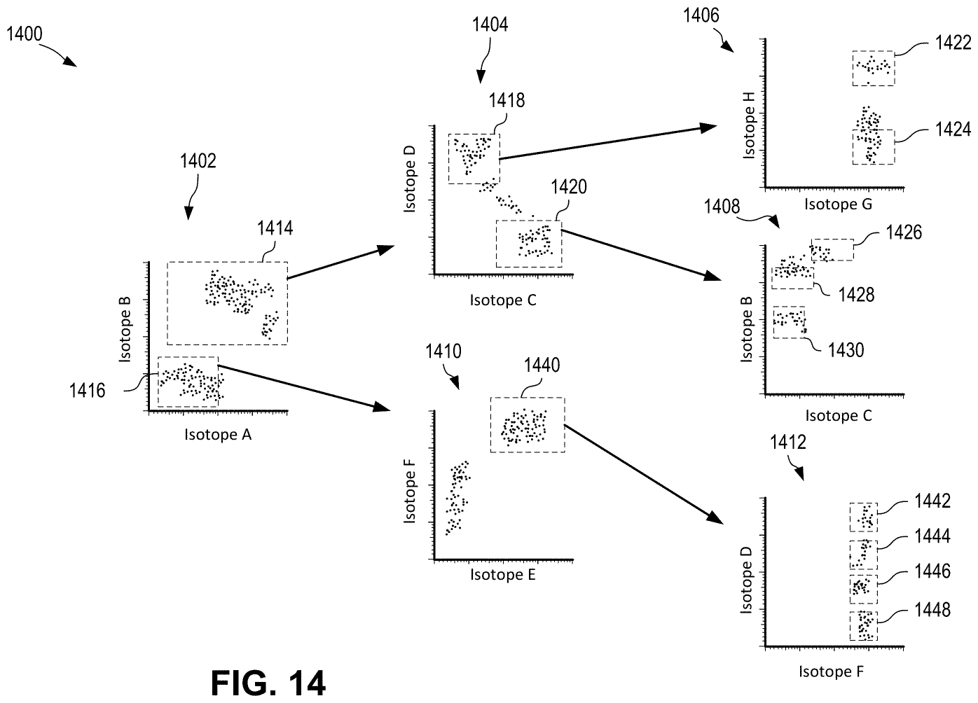

FIG. 14 is a schematic diagram depicting an example gating strategy 1400 for automatically analyzing elemental data according to certain aspects of the present disclosure. The example gating strategy 1400 includes three layers of gating, although any number of layers can be used. Various 2-dimensional spaces are used to depict cells or particles detected in a sample interrogated using elemental analysis. The higher the presence of an isotope or marker in conjunction with a cell or particle, the further right (on the x-axis) or up (on the y-axis) the dot signifying the cell or particle will appear in the space. The spaces depicted in FIG. 14 are labeled with isotopes on the x and y axes, although it is understood that each isotope can signify a particular marker, such as an antibody or a antibody's target. Additionally, compositions of isotopes can be used instead of individual isotopes along an axis, such as when a composition of isotopes is used to tag a particular marker. While these 2-dimensional spaces are depicted as dot-plots for illustrative purposes, the information can be stored and manipulated in any suitable fashion to achieve the desired gating strategies.

In a first layer, the elemental data from a sample is depicted in a 2-dimensional space 1402 with isotope A on the x-axis and isotope B on the y-axis. This space 1402 can include two gates 1414, 1416. Objects (e.g., cells or particles) associated with high values for isotope A and isotope B can fall within gate 1414, whereas objects associated with low values for isotope A and isotope B can fall within gate 1416. In some cases, objects within gate 1414 and/or objects within gate 1416 can be labelled as particular types of cells or objects.

In a second layer, the objects within gate 1414 can be plotted on space 1404, with the objects from gate 1416 being plotted on space 1410. Space 1404 can plot isotope C on the x-axis and isotope D on the y-axis. Space 1404 can be gated to separate objects with high values for isotope D and low values of isotope C into gate 1418 and objects with low values for isotope D and high values for isotope C into gate 1420. In space 1410, the objects from space 1416 can be plotted with isotope E on the x-axis and isotope F on the y-axis. Space 1410 can contain a single gate 1440, associated with objects having high values of both isotopes E and F. In some cases, objects within gates 1418, 1420, and/or 1440 can be labelled as particular types of cells or particle.

The objects within gates 1418, 1420, and/or 1440 can be passed along to a third layer. In the third layer, objects within gate 1418 are plotted on space 1406, objects within gate 1420 are plotted on space 1408, and objects within gate 1440 are plotted on space 1412. In space 1406, the objects are plotted with isotope G on the x-axis and isotope H on the y-axis. These objects can be gated with gate 1422 being associated with those objects having high values for both isotopes G and H, and gate 1424 being associated with those objects having high values for isotope H and low values for isotope H. Objects in gate 1422 and/or gate 1424 can be labelled as particular types of objects. In space 1408, the objects from space 1420 are plotted with isotope C on the x-axis and isotope B on the y-axis. These objects can be gated with gate 1426 being associated with those objects having high values for isotopes B and medium values for isotope C, gate 1428 being associated with those objects having medium-high values for isotope B and low values for isotope C, and gate 1430 being associated with those objects having medium values for isotope B and low values for isotope C. Objects in gates 1426, 1428, and/or 1430 can be labelled as particular types of objects. In space 1412, the objects from space 1440 are plotted with isotope F on the x-axis and isotope D on the y-axis. These objects can be gated with gate 1442 being associated with those objects having high values for isotopes F and D, gate 1444 being associated with those objects having medium-high values for isotope D and high values for isotope F, gate 1446 being associated with those objects having medium-low values for isotope D and high values for isotope F, and gate 1448 being associated with those objects having low values for isotopes D and F. Objects in gates 1442, 1444, 1446, and/or 1448 can be labelled as particular types of cells or objects.

Thus, a gating scheme can involve determining confined region(s) in a multi-dimensional space (e.g., a 2-dimensional space) in which data can be attributable to a particular result. A gating scheme can involve subsequently applying subsequent gate(s) to the data within a previous confined region based on a new multi-dimensional space (e.g., a different 2-dimensional space). The process can be repeated as necessary to reach the desired level of differentiation. Thus, successive levels of gating can help narrow down a particular cell type or object type based on the presence or absences of various markers. Since unique cell types or object types will have different combinations of surface markers and/or intracellular markers, gating strategies can be defined to identify these various cell types or particle types based on the presence or absence of various markers.

In some cases, instead of a confined region, cells or particles can be gated according to having marker expressions that are greater or less than a threshold number. For example, an indication of high expression for a given target (e.g., CD45) can be denoted as any expression that is at or greater a particular threshold. In some cases, different thresholds can be used for the same target on different levels of a gating scheme or for the same target on the same level of the gating scheme when originating from a different prior gate.

In an example, elemental data can be converted to target expression using mapping data. For a sample of cells, the data can be analyzed across a 2 dimensional space where target expression of CD45 is on a y axis and target expression of CD66b is on an x axis. A region in the top left corner of the space is indicative of high expression of CD45 and low or no expression of CD66b. The cells that fall into the top left corner can be further analyzed across a new space based on the target expression of CD56 and CD14. Cells falling into a lower left corner of that space can be indicative of little to no expression of either CD56 or CD14. The cells that fall into that region can be further analyzed across a new space based on the target expressions of CD19 on they axis and CD3 on the x axis. Cells that fall into a top left axis can be indicative of high CD19 expression and low or no CD3 expression. Cells that fall into that region can be identified as B cells. This example gating strategy can be denoted as CD45+CD66b−; CD56−CD14−; CD19+CD3−. In some cases, the cells in that region can be further analyzed along other dimensions to determine further characteristics about those cells.

Using the gating strategy notation from the example above, other gating strategies suitable for use with lyophilized antibody panels include the following strategies for identifying the following cell types. For CD8 T Cells (Total; CD161lo/−): CD45+CD66b−; CD19−CD20−; CD14−CD11c−; CD45+CD3+; CD3+TCRgd−; CD4−CD8+; CD8+CD161lo/−. For CD4 T Cells (Total): CD45+CD66b−; CD19−CD20−; CD14−CD11c−; CD45+CD3+; CD3+TCRgd−; CD4+CD8−. For Tregs: CD45+CD66b−; CD19−CD20−; CD14−CD11c−; CD45+CD3+; CD3+TCRgd−; CD4+CD8−; CD4+CCR4+; CD45RA−CD45RO+; CD25hiCD127lo/−. For Gamma-delta T Cells, CD4−CD8−: CD45+CD66b−; CD19−CD20−; CD14−CD11c−; CD45+CD3+; CD4−CD8−; CD3+TCRgd+. For Total B Cells: CD45+CD66b−; CD56−CD14−; CD19+CD3−. For Total NK Cells: CD45+CD66b−; CD19−CD20−; CD3−CD14−; CD45RA+CD123−; CD45+CD56+. For Neutrophils: CD45loCD66b+; CD294−CD16+. For Total Monocytes: CD45+CD66b−; CD19−CD20−; CD3−CD56−; CD11c+HLA−DR+; CD14+/−CD11c+. For Plasmacytoid Dendritic Cells: CD45+CD66b−; CD19−CD20−; CD3−CD14−; HLA−DR+CD56+/−; CD123+CD11c−. For CD8 T Cells, Naïve: CD45+CD66b−; CD19−CD20−; CD14−CD11c−; CD45+CD3+; CD3+TCRgd−; CD4−CD8+; CD8+CD161lo/−; CD8+CCR7hi; CD45RA+CD45RO−. For CD4 T Cells, Naïve: CD45+CD66b−; CD19−CD20−; CD14−CD11c−; CD45+CD3+; CD3+TCRgd−; CD4+CD8−; CD4+CCR7hi; CD45RA+CD45RO−. For Th1-like: CD45+CD66b−; CD19−CD20−; CD14−CD11c−; CD45+CD3+; CD3+TCRgd−; CD4+CD8−; CD4+CXCR5−; CD4+CCR4−; CD45RA−CD45RO+; CXCR3+CCR6−. For MAIT/NKT CD4− Cells: CD45+CD66b−; CD19−CD20−; CD14−CD11c−; CD45+CD3+; CD3+CD4−; CD28+CD161hi. For Naive B cells: CD45+CD66b−; CD56−CD14−; CD19+CD3−; CD19+CD27+. For Early NK Cells: CD45+CD66b−; CD19−CD20−; CD3−CD14−; CD45RA+CD123−; CD45+CD56+; CD56+CD57−. For Eosinophils: CD45loCD66b+; CD294+CD16−. For Classical Monocytes: CD45+CD66b−; CD19−CD20−; CD3−CD56−; CD11c+HLA−DR+; CD14+/−CD11c+; CD38+CD14hi. For Myeloid Dendritic Cells: CD45+CD66b−; CD19−CD20−; CD3−CD14−; HLA−DR+CD56+/−; CD123−CD11c+; CD11c+CD38+. For CD8 T Cells, Central Memory: CD45+CD66b−; CD19−CD20−; CD14−CD11c−; CD45+CD3+; CD3+TCRgd−; CD4−CD8+; CD8+CD161lo/−; CD8+CCR7hi; CD45RA−CD45RO+. For CD4 T Cells, Central Memory: CD45+CD66b−; CD19−CD20−; CD14−CD11c−; CD45+CD3+; CD3+TCRgd−; CD4+CD8−; CD4+CCR7hi; CD45RA−CD45RO+. For Th2-like: CD45+CD66b−; CD19−CD20−; CD14−CD11c−; CD45+CD3+; CD3+TCRgd−; CD4+CD8−; CD4+CXCR5−; CD45RA−CCR4+; CXCR3−CCR6−. For Memory B cells: CD45+CD66b−; CD56−CD14−; CD19+CD3−; CD19+CD27+. For Late NK Cells: CD45+CD66b−; CD19−CD20−; CD3−CD14−; CD45RA+CD123−; CD45+CD56+; CD56+CD57+. For Basophils: CD45+CD66b−; CD19−CD20−; CD3−CD56−; HLA−DR−CD11c−; CD123+CD294+. For Intermediate Monocytes: CD45+CD66b−; CD19−CD20−; CD3−CD56−; CD11c+HLA−DR+; CD14+/−CD11c+; CD38lo/−CD14int. For CD8 T cells, Effector Memory: CD45+CD66b−; CD19−CD20−; CD14−CD11c−; CD45+CD3+; CD3+TCRgd−; CD4−CD8+; CD8+CD161lo/−; CD8+CCR7lo/−; CD8+CD27+. For CD4 T cells, Effector Memory: CD45+CD66b−; CD19−CD20−; CD14−CD11c−; CD45+CD3+; CD3+TCRgd−; CD4+CD8−; CD4+CCR7lo/−; CD45RA−CD45RO+; CD45RO+CD27+. For Th17-like: CD45+CD66b−; CD19−CD20−; CD14−CD11c−; CD45+CD3+; CD3+TCRgd−; CD4+CD8−; CD4+CXCR5−; CD45RA−CCR4+; CXCR3−CCR6+. For Plasmablasts: CD45+CD66b−; CD56−CD14−; CD19+CD3−; CD19+CD27+; CD38+CD20−. For Nonclassical Monocytes: CD45+CD66b−; CD19−CD20−; CD3−CD56−; CD11c+HLA−DR+; CD14+/−CD11c+; CD38−CD14−. For CD8 T cells, Terminal Effector: CD45+CD66b−; CD19−CD20−; CD14−CD11c−; CD45+CD3+; CD3+TCRgd−; CD4−CD8+; CD8+CD161lo/−; CD8+CCR7lo/−; CD8+CD27−. For CD4 T cells, Terminal Effector: CD45+CD66b−; CD19−CD20−; CD14−CD11c−; CD45+CD3+; CD3+TCRgd−; CD4+CD8−; CD4+CCR7lo/−; CD45RA−CD45RO+; CD45RO+CD27−.

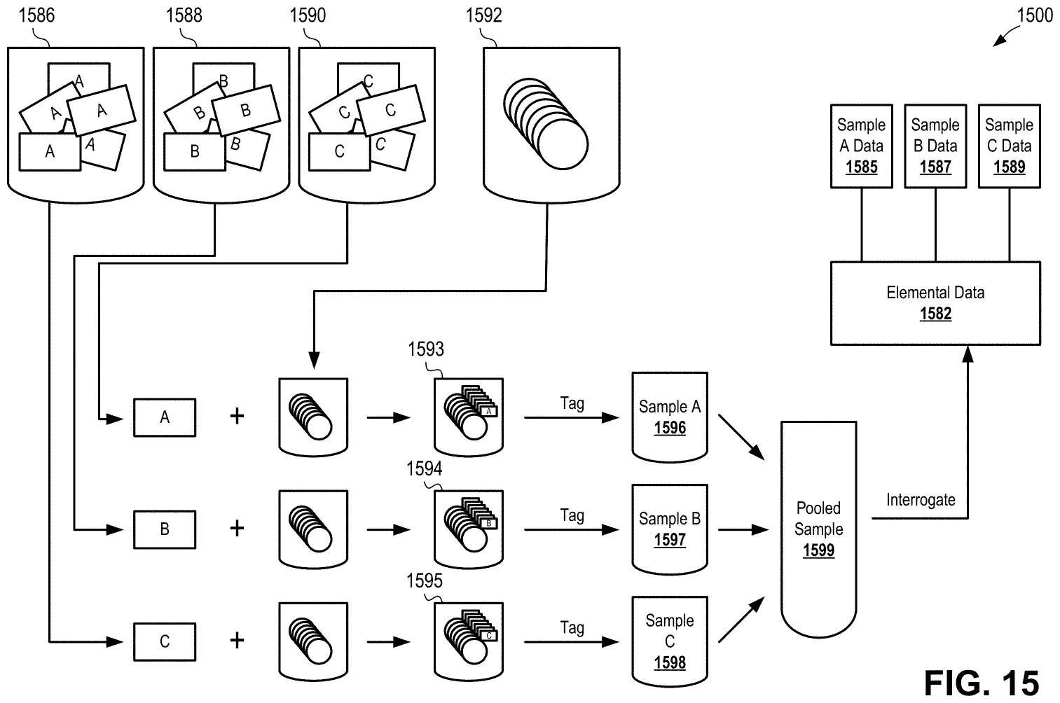

FIG. 15 is a schematic diagram depicting a technique 1500 for sample-labeling barcoding reagents and analyzing a set of samples according to certain aspects of the present disclosure. A set of barcoding reagents 1592 is provided. The set of barcoding reagents 1592 can include any number of barcoding reagents (e.g., like barcoding reagent 208 of FIG. 2) for any number of assays, optionally element-tagged with assay barcodes. The set of barcoding reagents 1592 can include barcoding reagents with various different capture antibodies and/or assay-specific biomolecules. Three sets of sample barcodes 1586, 1588, 1590 can be provided. While FIG. 15 describes the use of three sets of sample barcodes 1586, 1588, 1590, any number of sets of sample barcodes can be used, which can be determined in advance by the number of available sets of sample barcodes and/or the number of different samples being assayed. Each of the sets of sample barcodes 1586, 1588, 1590 can contain a number of identical sample barcodes, such that the sample barcodes of a first set of sample barcodes 1586 are all identical and all unique from a second set of sample barcodes 1588, which are all themselves identical and all unique from a third set of sample barcodes 1590, which are all themselves identical. The three sets of sample barcodes 1586, 1588, 1590 and/or the set of barcoding reagents 1592 can be provided as a kit or as part of a kit including a lyophilized panel.

The sets of sample barcodes 1586, 1588, 1590 can be individually combined with aliquots of the set of barcoding reagents 1592. Each aliquot of the set of barcoding reagents 1592 can contain a mixture of all different types of barcoding reagents from the set of barcoding reagents 1592. As a result of combining the sets of sample barcodes 1586, 1588, 1590 with the set of barcoding reagents 1592 in individual aliquots, three sets of sample-encoded barcoding reagents 1593, 1594, 1595 can be created. The sample barcodes 1586, 1588, 1590 can be coupled to barcoding reagents 1592 or can be merely in admixture with the barcoding reagents 1592. Thus, a first set of sample-encoded barcoding reagents 1593 can contain barcoding reagents that are all tagged with or in admixture with the sample barcode from the first set of sample barcodes 1586; a second set of sample-encoded barcoding reagents 1594 can contain barcoding reagents that are all tagged with or in admixture with the sample barcode from the second set of sample barcodes 1588; and a third set of sample-encoded barcoding reagents 1595 can contain barcoding reagents that are all tagged with or in admixture with the sample barcode from the third set of sample barcodes 1590. When the sample barcodes 1586, 1588, 1590 are used to tag the barcoding reagents 1592, excess sample barcodes can be washed from each mixture.

Each of the sets of sample-encoded barcoding reagents 1593, 1594, 1595 can be mixed with respective samples 1596, 1597, 1598 to tag the samples 1596, 1597, 1598. After washing unbound barcoding reagent (and optionally, unbound sample barcodes), the samples 1596, 1597, 1598 can be combined into a pooled sample 1599.

The pooled sample 1599 can be interrogated using elemental analysis, such as using an elemental analyzer (e.g., elemental analyzer 424 of FIG. 4). The resultant elemental data 1582 can be analyzed to detect the presence of the elemental tags associated with the sample barcodes from the three sets of sample barcodes 1586, 1588, 1590, and thus identify the individual sample (e.g., sample 1596, 1597, 1598, respectively) to which the sample barcode is associated. An automated analyzer or processor (e.g., elemental data processor 430 of FIG. 4) can automatically split the elemental data 1582 captured by the elemental analyzer into Sample A Data 1585 (e.g., elemental data associated with sample A 1596, which was tagged with sample barcodes from the set of sample barcodes 1586), Sample B Data 1587 (e.g., elemental data associated with sample B 1597, which was tagged with sample barcodes from the set of sample barcodes 1588), and Sample C Data 1589 (e.g., elemental data associated with sample C 1598, which was tagged with sample barcodes from the set of sample barcodes 1590).

Thus, many samples can be combined and analyzed simultaneously, which can improve overall interrogation efficiency and can help improve reliability of data between the samples since they were all interrogated during the same run.

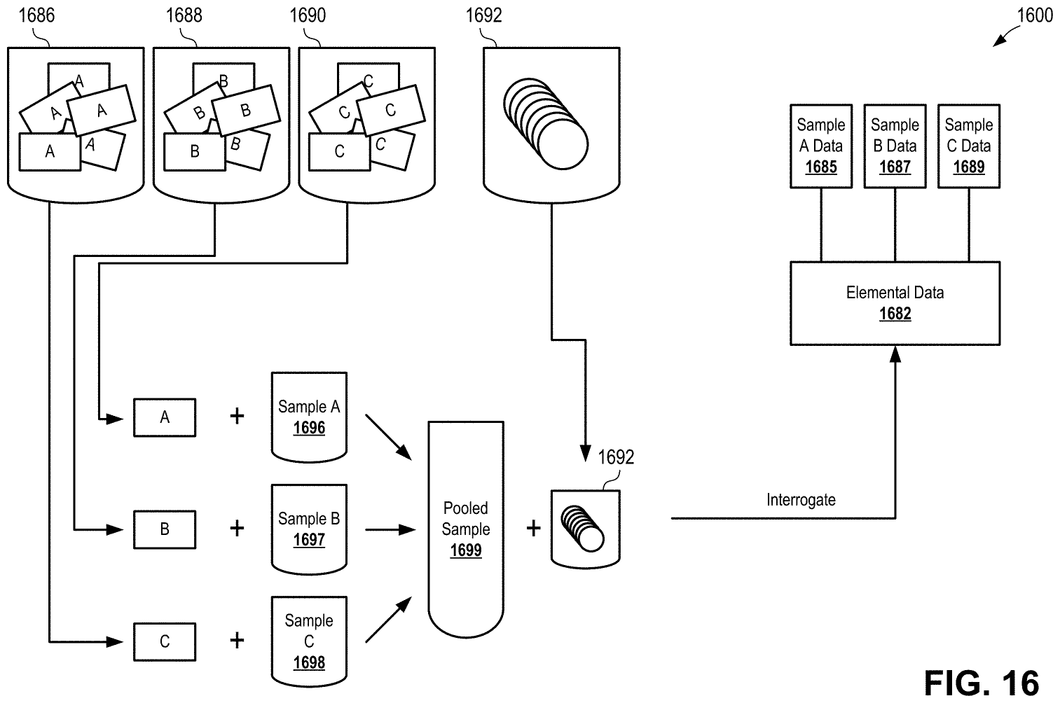

FIG. 16 is a schematic diagram depicting a technique 1600 for sample-labeling and analyzing a set of samples according to certain aspects of the present disclosure. A set of barcoding reagents 1692 is provided. The set of barcoding reagents 1692 can include any number of barcoding reagents (e.g., like barcoding reagent 208 of FIG. 2) for any number of assays, optionally element-tagged with assay barcodes. The set of barcoding reagents 1692 can include barcoding reagents with various different capture antibodies and/or assay-specific biomolecules. Three sets of sample barcodes 1686, 1688, 1690 can be provided. While FIG. 16 describes the use of three sets of sample barcodes 1686, 1688, 1690, any number of sets of sample barcodes can be used, which can be determined in advance by the number of available sets of sample barcodes and/or the number of different samples being assayed. Each of the sets of sample barcodes 1686, 1688, 1690 can contain a number of identical sample barcodes, such that the sample barcodes of a first set of sample barcodes 1686 are all identical and all unique from a second set of sample barcodes 1688, which are all themselves identical and all unique from a third set of sample barcodes 1690, which are all themselves identical. The three sets of sample barcodes 1686, 1688, 1690 and/or the set of barcoding reagents 1692 can be provided as a kit or as part of a kit including a lyophilized panel.

The sets of sample barcodes 1686, 1688, 1690 can be individually combined with respective samples 1696, 1697, 1698. In some cases, such as if the sample barcodes are designed to bind to cells or particles of the sample itself, the samples 1696, 1697, 1698 can be washed to remove any unbound sample barcodes. Then, the samples 1696, 1697, 1698 can be pooled together into a pooled sample 1699 that can be combined with the set of barcoding reagents 1692. Then, unbound barcoding reagent can be washed and the pooled sample 169 can be interrogated using elemental analysis, such as using an elemental analyzer (e.g., elemental analyzer 424 of FIG. 4). The resultant elemental data 1682 can be analyzed to detect the presence of the elemental tags associated with the sample barcodes from the three sets of sample barcodes 1686, 1688, 1690, and thus identify the individual sample (e.g., sample 1696, 1697, 1698, respectively) to which the sample barcode is associated. An automated analyzer or processor (e.g., elemental data processor 430 of FIG. 4) can automatically split the elemental data 1682 captured by the elemental analyzer into Sample A Data 1685 (e.g., elemental data associated with sample A 1696, which was tagged with sample barcodes from the set of sample barcodes 1686), Sample B Data 1687 (e.g., elemental data associated with sample B 1697, which was tagged with sample barcodes from the set of sample barcodes 1688), and Sample C Data 1689 (e.g., elemental data associated with sample C 1698, which was tagged with sample barcodes from the set of sample barcodes 1690).

Thus, many samples can be combined together to be assayed (e.g., assayed using barcoding reagents from the set of barcoding reagents 1692) and analyzed simultaneously, which can improve assay efficiency, overall interrogation efficiency, and can help improve reliability of data between the samples since they were all assayed and interrogated during the same run.

In some cases, each combination of sample barcodes from the three sets of sample barcodes 1686, 1688, 1690 and their respective samples 1696, 1697, 1698 can be combined individually with aliquots of the set of barcoding reagents 1692 prior the being washed and then pooled into pooled sample 1699, similar to the technique 1500 described with reference to FIG. 15.

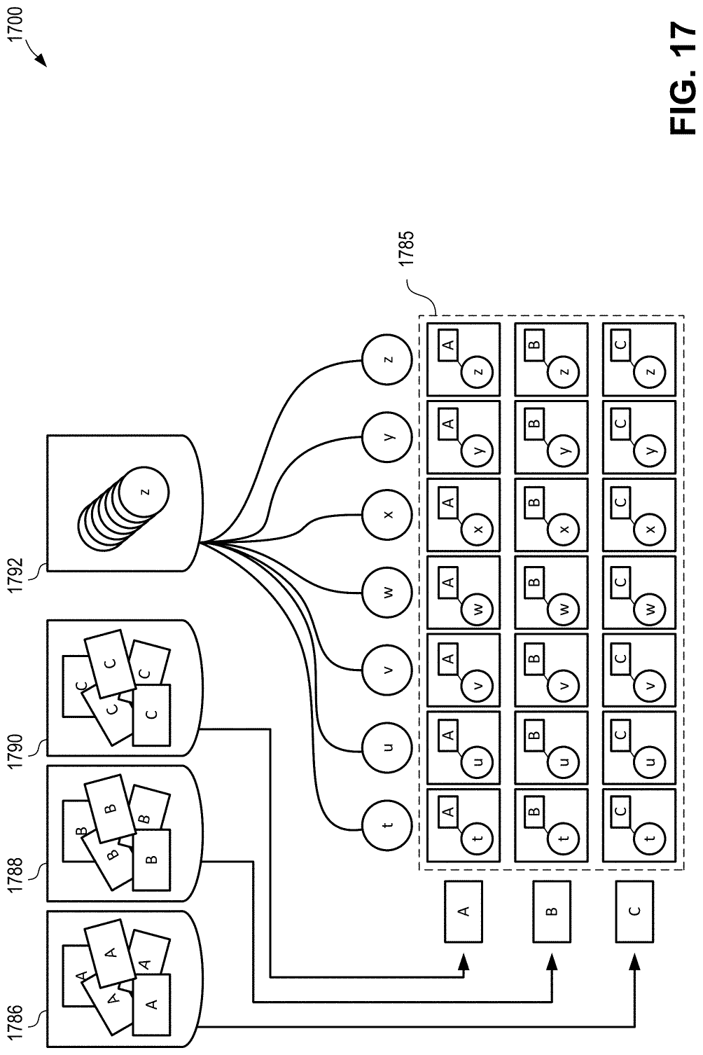

FIG. 17 is a schematic diagram depicting a technique 1700 for preparing a set of pre-configured sample-barcode-labeled barcoding reagents according to certain aspects of the present disclosure. A set of barcoding reagents 1792 is provided. The set of barcoding reagents 1792 can include any number of barcoding reagents (e.g., like barcoding reagent 208 of FIG. 2) for any number of assays, optionally element-tagged with assay barcodes. The set of barcoding reagents 1792 can include barcoding reagents with various different capture antibodies and/or assay-specific biomolecules. Three sets of sample barcodes 1786, 1788, 1790 can be provided. While FIG. 17 describes the use of three sets of sample barcodes 1786, 1788, 1790, any number of sets of sample barcodes can be used, which can be determined in advance by the number of available sets of sample barcodes and/or the number of different samples being assayed. Each of the sets of sample barcodes 1786, 1788, 1790 can contain a number of identical sample barcodes, such that the sample barcodes of a first set of sample barcodes 1786 are all identical and all unique from a second set of sample barcodes 1788, which are all themselves identical and all unique from a third set of sample barcodes 1790, which are all themselves identical. The three sets of sample barcodes 1786, 1788, 1790 and/or the set of barcoding reagents 1792 can be provided as a kit or as part of a kit including a lyophilized panel.

As depicted in FIG. 17, the set of barcoding reagents 1792 contains seven different types of barcoding reagents (e.g., reagents t through z). Each different type of barcoding reagent represents a barcoding reagent having a unique assay barcode and a unique capture antibody or assay-specific biomolecule. Each different type of barcoding reagent of the set of barcoding reagents 1792 can be distributed into a number of wells down respective columns of a well plate 1785. Thus, each well down a column shares the same type of barcoding reagent, whereas each well across a row contains different barcoding reagents.

Each set of sample barcodes 1786, 1788, 1790 can be distributed across respective rows of the well plate 1785. Thus, each well across a row shares the same sample barcode, whereas each well down a column contains different sample barcodes.

As a result of the combination of the sample barcodes and barcoding reagents in the wells of the well plate 1785, each well will contain a unique combination of sample barcodes and barcoding reagents (e.g., t:A; u:B, through z:C). The sample barcodes can optionally bind to the barcoding reagents within the same well, or can simply be maintained in mixture for future binding to a sample cell or particle.

Thus, a set of unique combinations of sample barcodes and barcoding reagents can be created. Depending on the needs of a user, some or all of the set of unique combinations of sample barcodes and barcoding reagents can be supplied to one or more samples to label the samples and perform assays as desired.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art.

As used below, any reference to a series of examples is to be understood as a reference to each of those examples disjunctively (e.g., “Examples 1-4” is to be understood as “Examples 1, 2, 3, or 4”).

Example 1 is a panel for elemental analysis, comprising: a plurality of conjugated antibodies, wherein each of the plurality of conjugated antibodies is tagged with a distinct element tag, wherein each distinct element tag is distinguishable based on its isotopic composition, and wherein the plurality of conjugated antibodies is in a lyophilized mixture.

Example 2 is the panel of example(s) 1, wherein the plurality of conjugated antibodies includes two or more antibodies from the list comprising Cd45, CD45RA, CD45RO, Cd123, CD4, CD8a, CD11C, CD57, CXCR3, CD185, CD38, CD56, CD3, CD20, CD66b, HLA-DR, IgD, CD27, CD28, CD127, CD19, CD16, CD161, CD194, CD25, CD294, CD197, CD14, CCR6, and TCR δγ.

Example 3 is the panel of example(s) 1 or 2, wherein a majority of the conjugated antibodies are specific to a cell type in human peripheral blood.

Example 4 is the panel of example(s) 1-3, wherein a majority of the conjugated antibodies are specific to a cell surface marker.

Example 5 is the panel of example(s) 1-4, wherein the plurality of conjugated antibodies comprises ten or more conjugated antibodies in the lyophilized mixture.

Example 6 is the panel of example(s) 1-5, wherein each distinct element tag comprises a plurality of elemental atoms of one isotope.

Example 7 is the panel of example(s) 1-6, wherein at least two conjugated antibodies of the plurality of conjugated antibodies are tagged with distinct element tags having different isotopes of a single element.

Example 8 is the panel of example(s) 1-7, further comprising a biomolecule coupled to an additional element tag, wherein the biomolecule is not an antibody, and wherein the additional element tag is distinguishable from each distinct element tag based on its isotopic composition.

Example 9 is the panel of example(s) 1-8, further comprising a non-antibody metal-containing moiety comprising a metal isotope that is distinguishable from each distinct element tag based on its isotopic composition.

Example 10 is the panel of example(s) 1-9, wherein each distinct element tag comprises a metal element having an atomic mass greater than 80 amu.

Example 11 is the panel of example(s) 1-10, wherein each distinct element tag comprises a chelated metal.

Example 12 is the panel of example(s) 1-11, wherein each distinct element tag comprises an element that is not endogenous to human peripheral blood.

Example 13 is the panel of example(s) 1-12, wherein the panel has a moisture content that is at or less than 5% by weight.

Example 14 is the panel of example(s) 1-12, wherein the panel has a moisture content that is at or less than 3% by weight.

Example 15 is the panel of example(s) 1-12, wherein the panel has a moisture content that is at or less than 1% by weight.

Example 16 is the panel of example(s) 1-12, wherein the panel has a moisture content that is at or between 0.05 and 1% by weight.

Example 17 is the panel of example(s) 1-16, further comprising a lyophilized intercalator, wherein the lyophilized intercalator is included in the lyophilized mixture.

Example 18 is the panel of example(s) 1-17, further comprising a lyophilized calibration material, wherein the lyophilized calibration material comprises known quantities of one or more known isotope(s), and wherein the lyophilized calibration material is included in the lyophilized mixture.

Example 19 is the panel of example(s) 1-18, further comprising a supplemental reagent, wherein the supplemental reagent is usable for conducting an assay using the plurality of conjugated antibodies, wherein the supplemental reagent is lyophilized, and wherein the supplemental reagent is included in the lyophilized mixture.

Example 20 is an assay kit for use with elemental analysis, comprising: a hermetically sealed container; and the panel of example(s) 1-19, wherein the lyophilized mixture of the panel is stored within the hermetically sealed container.

Example 21 is the assay kit of example(s) 20, wherein the hermetically sealed container includes an internal atmosphere of an inert gas or dry air.

Example 22 is the assay kit of example(s) 20 or 21, wherein the plurality of conjugated antibodies includes a first antibody specific to a cell surface marker and second antibody specific to an intracellular target.

Example 23 is the assay kit of example(s) 20-22, further comprising: an additional hermetically sealed container; and an additional antibody panel comprising at least one additional conjugated antibody tagged with an additional distinct element tag that is distinguishable from each distinct element tag based on its isotopic composition, wherein the additional conjugated antibody is lyophilized and stored within the additional hermetically sealed container.

Example 24 is the assay kit of example(s) 23, wherein the plurality of conjugated antibodies includes antibodies specific to one or more cell surface markers, and wherein the additional conjugated antibody is specific to an intracellular target.

Example 25 is the assay kit of example(s) 20-24, further comprising an intercalator comprising an additional distinct element tag that is distinguishable from each distinct element tag based on its isotopic composition.

Example 26 is the assay kit of example(s) 20-25, further comprising a plurality of sample barcoding reagents for labelling a plurality of samples, wherein each of the plurality of sample barcoding reagents comprises a distinct combination of isotopes.

Example 27 is the assay kit of example(s) 26, further comprising a plurality of containers, wherein each of the plurality of sample barcoding reagents is contained within different containers of the plurality of containers.

Example 28 is the assay kit of example(s) 26 or 27, wherein each of the plurality of sample barcoding reagents binds to a majority of cells in a sample.

Example 29 is the assay kit of example(s) 26-28, wherein each of the plurality of sample barcoding reagents comprise element tags functionalized to covalently bind on or within cells of a sample.

Example 30 is the assay kit of example(s) 26-29, wherein each of the plurality of sample barcoding reagents comprises sample barcoding antibodies that specifically bind a target present across a majority of cells in a sample or that together binds multiple targets across a majority of cells in the sample.

Example 31 is the assay kit of example(s) 30, wherein each of the sample barcoding antibodies specifically binds one or more of CD45, CD298 and b2m.

Example 32 is the assay kit of example(s) 30 or 31, wherein the element tags of the sample barcoding antibodies provide a weaker signal than the element tags of the majority of other antibodies in the panel when analyzed using an elemental analyzer.

Example 33 is the assay kit of example(s) 26-32, wherein the distinct combination of isotopes comprises cadmium.

Example 34 is the assay kit of example(s) 26-33, wherein the distinct combination of isotopes comprises platinum in cisplatin.

Example 35 is the assay kit of example(s) 26-34, wherein each of the plurality of sample barcoding reagents comprises a set of sample barcoding antibodies, wherein each sample barcoding antibody comprises all of the isotopes of the distinct combination of isotopes.

Example 36 is the assay kit of example(s) 26-35, wherein each of the plurality of sample barcoding reagents is capable of barcoding live cells, and wherein each of the plurality of sample barcoding reagents is non-toxic to the live cells.

Example 37 is the assay kit of example(s) 20-36, further comprising assay barcoding reagents comprising additional antibodies for detecting different analytes, wherein each assay barcoding reagent comprises a distinct combination of isotopes.

Example 38 is the assay kit of example(s) 37, wherein each assay barcoding reagent is an assay barcoding bead comprising the distinct combination of isotopes.

Example 39 is the assay kit of example(s) 38, wherein the assay barcoding reagents are included in the lyophilized mixture of the panel.

Example 40 is the assay kit of example(s) 38 or 39, wherein each assay barcoding bead comprises a unique combination of isotopes present within an interior of the assay barcoding bead.

Example 41 is the assay kit of example(s) 37-40, wherein the assay barcoding reagents comprise at least ten assay barcoding reagents for barcoding at least ten different analytes, and wherein the assay barcoding reagents are provided in admixture.

Example 42 is the assay kit of example(s) 37-41, wherein the different analytes are free analytes in human peripheral blood.

Example 43 is the assay kit of example(s) 37-42, further comprising a combination of reporter antibodies that specifically bind the different analytes, wherein each reporter antibody comprises an element tag detectable by elemental analysis.

Example 44 is the assay kit of example(s) 43, wherein each of the element tags of the combination of reporter antibodies comprises an isotopically identical element detectable by elemental analysis.

Example 45 is the assay kit of example(s) 37-44, wherein each assay barcoding reagent is functionalized to attach to a sample barcode comprising a sample barcoding composition of isotopes.

Example 46 is the assay kit of example(s) 45, further comprising the sample barcode comprising the sample barcoding composition of isotopes.

Example 47 is the assay kit of example(s) 45 or 46, wherein the sample barcode can bind to cells of a sample stained with the lyophilized panel.

Example 48 is the assay kit of example(s) 20-47, further comprising barcoding reagents, wherein each barcoding reagent comprises an assay barcoding composition of isotopes and a sample barcoding composition of isotopes, wherein each distinct assay barcoding composition of isotopes is associated with a distinct analyte, and wherein each distinct sample barcoding composition of isotopes is associated with a distinct sample.

Example 49 is the assay kit of example(s) 48, wherein each barcoding reagent is a bead, and wherein the sample barcoding composition of isotopes is located within an interior of the bead.

Example 50 is the assay kit of example(s) 48, wherein each barcoding reagent is a bead, and wherein the sample barcoding composition of isotopes is located on a surface of the bead.

Example 51 is the assay kit of example(s) 20-50, further comprising an anticoagulant.

Example 52 is the assay kit of example(s) 20-51, further comprising a calibration material, wherein the calibration material comprises a known quantity of a known isotope.

Example 53 is an assay kit for use with elemental analysis, comprising: a plurality of hermetically sealed containers; and the panel of example(s) 1-19, wherein the lyophilized mixture of the panel is distributed across the plurality of hermetically sealed containers.

Example 54 is the assay kit of example(s) 53, further comprising a plurality of sample barcoding reagents for labelling a plurality of samples, wherein each of the plurality of sample barcoding reagents comprises a distinct combination of isotopes, and wherein each of the plurality of sample barcoding reagents is contained within different containers of the plurality of hermetically sealed containers.

Example 55 is a barcoding system, comprising: a barcoding reagent comprising an assay barcode, wherein the assay barcode comprises a composition of isotopes associated with a target analyte, wherein the composition of isotopes is distinguishable through elemental analysis, wherein the barcoding reagent comprises one of a plurality of sample barcodes or is functionalized to bind to at least one of the plurality of sample barcodes, wherein each sample barcode of the plurality of sample barcodes comprises a unique additional composition of isotopes distinguishable from the assay barcode composition of isotopes through elemental analysis, wherein each sample barcode of the plurality of sample barcodes can be associated with a distinct sample. The barcoding system may optionally further comprise a panel from example 1 or a related example.

Example 56 is the system of example(s) 55, wherein the barcoding reagent is a bead, and optionally wherein the sample barcode is present in the interior of the bead.

Example 57 is the system of example(s) 55, wherein the barcoding reagent is a bead, and wherein a surface of the bead is functionalized to bind to the plurality of sample barcodes.

Example 58 is the system of example(s) 55-57, wherein the barcoding reagent is a bead, and wherein a surface of the bead comprises the one of the plurality of sample barcodes.

Example 59 is the system of example(s) 55-58, wherein the barcoding reagent is functionalized to bind to the plurality of sample barcodes, and optionally wherein the system further comprises each sample barcode of the plurality of sample barcodes in separate containers.

Example 60 is the system of example(s) 55-59, further comprising a sample barcoding reagent comprising at least one of the plurality of sample barcodes, and optionally wherein the sample barcoding reagent can bind both the barcoding reagent and cells of a sample.

Example 61 is the system of example(s) 55-60, wherein the barcoding reagent is a bead, and wherein the assay barcode is present in the interior of the bead, and optionally wherein the interior of the bead comprises a solid metal core, metal chelating polymer interior, nanocomposite interior, or hybrid interior.

Example 62 is the system of example(s) 56-58 or 61, wherein the bead has a solid metal core and a polymer surface.

Example 63 is the system of example(s) 62, wherein the polymer surface is bound to an antibody that binds to the target analyte.

Example 64 is the system of example(s) 63, wherein the target analyte is a free analyte present in blood.

Example 65 is the system of example(s) 55-64, further comprising a reporter antibody that specifically binds the target analyte and comprises an elemental tag or a combination of high and low intensity element tag(s).

Example 66 is the system of example(s) 65, wherein the assay barcoding reagents comprise at least ten assay barcoding reagents for barcoding at least ten different analytes, and optionally wherein a plurality of separate mixtures of the assay barcoding reagents each comprise an isotopically distinguishable sample barcode. Example 67 is the system of example(s) 65 or 66, further comprising reporter biomolecules that specifically bind the target analytes of the assay barcoding reagents, wherein the reporter biomolecules comprise each comprise an affinity reagent or oligonucleotides, and wherein each reporter biomolecule comprises an element tag or a combination of a high and low signal element tag.

Example 68 is the system of example(s) 65-67, wherein at least some of the reporter biomolecules that specifically bind different target analytes comprise the same element tag(s).

Example 69 is a method, comprising: providing a plurality of antibodies; conjugating each of the plurality of antibodies with a distinct element tag, wherein each distinct element tag is distinguishable based on its isotopic composition, and wherein each of the plurality of antibodies is distinguishable by its distinct element tag; mixing the plurality of conjugated antibodies together into an admixture; and lyophilizing the admixture.

Example 70 is the method of example(s) 69, further comprising spin filtering the plurality of conjugated antibodies.

Example 71 is the method of example(s) 69 or 70, further comprising: selecting an interrogation scheme for interrogating a sample; and selecting the plurality of antibodies based on the selected interrogation scheme.

Example 72 is the method of example(s) 69-71, wherein providing the plurality of antibodies includes providing two or more antibodies from the list including Cd45, CD45RA, CD45RO, Cd123, CD4, CD8a, CD11C, CD57, CXCR3, CD185, CD38, CD56, CD3, CD20, CD66b, HLA-DR, IgD, CD27, CD28, CD127, CD19, CD16, CD161, CD194, CD25, CD294, CD197, CD14, CCR6, and TCR δγ.

Example 73 is the method of example(s) 69-72, wherein each of the plurality of antibodies is specific to a cell type in human peripheral blood.

Example 74 is the method of example(s) 69-73, wherein each of the plurality of antibodies is specific to a cell surface marker.

Example 75 is the method of example(s) 69-74, wherein mixing the plurality of conjugated antibodies together comprises mixing together ten or more antibodies.

Example 76 is the method of example(s) 69-75, wherein each distinct element tag comprises a plurality of elemental atoms of one isotope.

Example 77 is the method of example(s) 69-76, wherein at least two of the distinct element tags have different isotopes of a single element.

Example 78 is the method of example(s) 69-77, further comprising providing a biomolecule comprising an additional element tag, wherein the additional element tag is distinguishable from each distinct element tag based on its isotopic composition, wherein the biomolecule is not an antibody, and wherein mixing the plurality of conjugated antibodies together further comprises mixing the biomolecule with the plurality of conjugated antibodies.

Example 79 is the method of example(s) 69-78, wherein each distinct element tag comprises a metal element having an atomic mass greater than 80 amu.

Example 80 is the method of example(s) 69-79, wherein each distinct element tag comprises a chelated metal.

Example 81 is the method of example(s) 69-80, wherein each distinct element tag comprises an element that is not endogenous to human peripheral blood.

Example 82 is the method of example(s) 69-81, wherein lyophilizing the admixture comprises reducing the moisture content to at or less than 5% by weight.

Example 83 is the method of example(s) 69-82, further comprising mixing an intercalator into the admixture before lyophilizing the admixture, wherein the intercalator comprises an additional element tag that is distinguishable from each distinct element tag based on its isotopic composition.

Example 84 is the method of example(s) 69-83, further comprising mixing a calibration material into the admixture before lyophilizing the admixture, wherein the calibration material comprises a known quantity of a known isotope.

Example 85 is the method of example(s) 69-84, further comprising mixing a supplemental reagent into the admixture before lyophilizing the admixture, wherein the supplemental reagent is usable to facilitate conducting an assay using the plurality of conjugated antibodies.

Example 86 is the method of example(s) 69-85, wherein lyophilizing the mixture further comprises storing the lyophilized mixture in a hermetically sealed container with an internal atmosphere of an inert gas or dry air.

Example 87 is the method of example(s) 69-86, wherein the plurality of antibodies includes a first antibody specific to a cell surface marker and second antibody specific to an intracellular target.

Example 88 is the method of example(s) 69-87, further comprising: providing at least one additional antibody; conjugating the at least one additional antibody with at least one additional distinct element tag that is distinguishable from each distinct element tag based on its isotopic composition; lyophilizing the at least one additional antibody; and storing the lyophilized at least one additional antibody separately from the lyophilized admixture.

Example 89 is the method of example(s) 88, wherein the plurality of antibodies includes antibodies specific to one or more cell surface markers, and wherein the additional antibody is specific to an intracellular target.

Example 90 is the method of example(s) 69-89, further comprising providing a plurality of sample barcoding reagents for labelling a plurality of samples, wherein each of the plurality of sample barcoding reagents comprises a distinct combination of isotopes.

Example 91 is the method of example(s) 90, further comprising: providing a plurality of containers; and storing each of the plurality of sample barcoding reagents within different containers of the plurality of containers.

Example 92 is the method of example(s) 90 or 91, wherein each of the plurality of sample barcoding reagents binds to a majority of cells in a sample.

Example 93 is the method of example(s) 90-92, wherein each of the plurality of sample barcoding reagents comprise element tags functionalized to covalently or otherwise permanently bind on or within cells of a sample.

Example 94 is the method of example(s) 90-93, wherein each of the plurality of sample barcoding reagents comprises a sample barcoding antibody that specifically binds a target present across a majority of cells in a sample.

Example 95 is the method of example(s) 94, wherein each of the sample barcoding antibodies specifically binds one or more of CD45, CD298, and b2m.

Example 96 is the method of example(s) 94 or 95, wherein each of the sample barcoding antibodies specifically binds a target present in a sample selected to result in a mass signal of a single sample barcoding antibody being weaker than a mass signal of a majority of the plurality of conjugated antibodies.

Example 97 is the method of example(s) 90-96, wherein the distinct combination of isotopes comprises cadmium.

Example 98 is the method of example(s) 90-97, wherein the distinct combination of isotopes comprises platinum in cisplatin.

Example 99 is the method of example(s) 90-98, wherein each of the plurality of sample barcoding reagents comprises a set of sample barcoding antibodies, wherein each sample barcoding antibody comprises all of the isotopes of the distinct combination of isotopes.

Example 100 is the method of example(s) 90-99, wherein each of the plurality of sample barcoding reagents is capable of barcoding live cells, and wherein each of the plurality of sample barcoding reagents is non-toxic to the live cells.

Example 101 is the method of example(s) 69-100, further comprising: providing assay barcoding reagents comprising additional antibodies for detecting different analytes, wherein each assay barcoding reagent comprises a distinct combination of isotopes.

Example 102 is the method of example(s) 101, wherein each assay barcoding reagent is an assay barcoding bead comprising a distinct combination of isotopes.

Example 103 is the method of example(s) 102, wherein each assay barcoding bead comprises a unique combination of isotopes present within an interior of the assay barcoding bead.

Example 104 is the method of example(s) 101-103, wherein the assay barcoding reagents comprise at least ten assay barcoding reagents for barcoding at least ten different analytes, and wherein the assay barcoding reagents are provided in admixture.

Example 105 is the method of example(s) 101-104, wherein the different analytes are free analytes in human peripheral blood.

Example 106 is the method of example(s) 101-105, further comprising providing a combination of reporter antibodies that specifically bind the different analytes, wherein each reporter antibody comprises an element tag detectable by elemental analysis.

Example 107 is the method of example(s) 106, wherein each of the element tags of the combination of reporter antibodies comprises an isotopically identical element detectable by elemental analysis.

Example 108 is the method of example(s) 101-107, further comprising functionalizing each assay barcoding reagent to attach to a sample barcode comprising a sample barcoding composition of isotopes.

Example 109 is the method of example(s) 108, wherein the sample barcode binds cells of a sample to be assayed by the lyophilized admixture.

Example 110 is the method of example(s) 108 or 109, further comprising providing the sample barcode comprising the sample barcoding composition of isotopes.

Example 111 is the method of example(s) 69-110, further comprising providing barcoding reagents, wherein each barcoding reagent comprises an assay barcoding composition of isotopes and a sample barcoding composition of isotopes, wherein each distinct assay barcoding composition of isotopes is associated with a distinct analyte, and wherein each distinct sample barcoding composition of isotopes is associated with a distinct sample.

Example 112 is the method of example(s) 111, wherein each barcoding reagent is a bead, and wherein the sample barcoding composition of isotopes is located within an interior of the bead.

Example 113 is the method of example(s) 111 or 112, wherein each barcoding reagent is a bead, and wherein the sample barcoding composition of isotopes is located on a surface of the bead.

Example 114 is the method of example(s) 69-113, further comprising providing an anticoagulant.

Example 115 is the method of example(s) 69-114, further comprising titrating and diluting each of the plurality of conjugated antibodies to a predetermined concentration prior to mixing the plurality of conjugated antibodies.

Example 116 is the method of example(s) 69-115, further comprising mixing the plurality of conjugated antibodies with an excipient prior to lyophilizing the admixture.

Example 117 is the method of example(s) 116, wherein the excipient comprises a sugar and bovine serum albumin.

Example 118 is the method of example(s) 116 or 117, further comprising mixing the plurality of conjugated antibodies with a viability stain prior to lyophilizing the admixture.

Example 119 is the method of example(s) 118, wherein the viability stain is a rhodium intercalator.

Example 120 is a method, comprising: preparing a sample; providing a lyophilized antibody panel comprising a plurality of conjugated antibodies, wherein each of the plurality of conjugated antibodies is tagged with a distinct element tag, and wherein each distinct element tag is distinguishable based on its isotopic composition; perform a surface stain on cells of the sample using the lyophilized antibody panel; and interrogating the sample using elemental analysis to detect a presence of the distinct element tags.

Example 121 is the method of example(s) 120, wherein interrogating the sample using elemental analysis comprises processing the sample on an inductively coupled plasma mass spectrometer to detect a presence of the distinct element tags of the lyophilized antibody panel.

Example 122 is the method of example(s) 120 or 121, further comprising staining the sample of cells with a viability stain.

Example 123 is the method of example(s) 120-122, wherein the viability stain is provided as part of the lyophilized antibody panel.

Example 124 is the method of example(s) 123, wherein the viability stain is rhodium.

Example 125 is the method of example(s) 120-124, further comprising performing a FcR block on the sample of cells.

Example 126 is the method of example(s) 120-125, further comprising fixing the sample after performing the surface stain.

Example 127 is the method of example(s) 120-126, further comprising staining the sample with an intercalator, wherein the intercalator comprises an additional distinct element tag that is distinguishable from each distinct element tag based on its isotopic composition.

Example 128 is the method of example(s) 127, wherein staining the sample with the intercalator occurs after permeabilizing the sample.

Example 129 is the method of example(s) 120-128, further comprising permeabilizing the sample and performing an intracellular stain on the sample of cells using at least one additional antibody, wherein the at least one additional antibody is tagged with an additional distinct element tag that is distinguishable from each distinct element tag based on its isotopic composition.

Example 130 is the method of example(s) 120-129, wherein preparing the sample comprises collecting whole blood.

Example 131 is the method of example(s) 130, wherein preparing the sample comprises isolating peripheral blood mononuclear cells from the whole blood.

Example 132 is the method of example(s) 120-131, further comprising labelling the sample using a sample barcoding reagent, wherein the sample barcoding reagent comprises a distinct combination of isotopes usable to distinguish the sample barcoding reagent from an additional sample barcoding reagent.

Example 133 is the method of example(s) 132, wherein interrogating the sample comprises acquiring data through elemental analysis, identifying the sample barcoding reagent by the distinct combination of isotopes in the acquired data, and associating the acquired data with the sample.

Example 134 is the method of example(s) 133, wherein interrogating the sample further comprises mixing the sample with an additional sample prior to acquiring data through elemental analysis.

Example 135 is the method of example(s) 120-134, wherein performing a surface stain comprises: adding a suspension of the cells of the sample to the lyophilized antibody panel or a resuspension of the lyophilized antibody panel; and removing unbound antibodies.

Example 136 is the method of example(s) 120-135, further comprising: providing assay barcoding reagents comprising additional antibodies for detecting different analytes, wherein each assay barcoding reagent comprises a distinct combination of isotopes; and mixing the assay barcoding reagent with the sample and removing unbound antibodies before interrogating the sample.

Example 137 is the method of example(s) 136, wherein the sample comprises blood plasma, and wherein the different analytes are free analytes within the blood plasma.

Example 138 is the method of example(s) 136 or 137, wherein providing the assay barcoding reagents and providing the lyophilized antibody panel occur by providing an admixture of the lyophilized antibody panel and the assay barcoding reagents.

Example 139 is the method of example(s) 136-138, wherein preparing the sample comprises collecting whole blood.

Example 140 is the method of example(s) 139, wherein preparing the sample further comprises isolating peripheral blood mononuclear cells and blood plasma, wherein performing the surface stain comprises mixing the lyophilized antibody panel with the peripheral blood mononuclear cells and removing unbound antibodies; and wherein mixing the assay barcoding reagents with the sample comprises mixing the assay barcoding reagents with the blood plasma and removing unbound antibodies.

Example 141 is the method of example(s) 120-140, wherein interrogating the sample further comprises automatically identifying cell viability.

Example 142 is the method of example(s) 120-141, wherein interrogating the sample further comprises automatically identifying cell populations.

Example 143 is the method of example(s) 142, wherein interrogating the sample further comprises identifying characteristics of automatically identified cell populations.

Example 144 is the method of example(s) 143, wherein interrogating the sample further comprises comparing the identified characteristics across the identified cell populations or comparing the identified characteristics associated with one of the identified cell populations with additional characteristics identified associated with the same one of the identified cell populations from an additional sample.

Example 145 is the method of example(s) 143 or 144, wherein identifying characteristics of automatically identified cell populations comprises determining an abundance of one or more targets on or in cells of the identified cell populations.

Example 146 is the method of example(s) 143-145, wherein the identified characteristics comprise a percentage of cells in the cell populations.

Example 147 is the method of example(s) 120-146, wherein interrogating the sample further comprise generating at least one of a histogram, a 2D dot plots, and a tSNE graph based on known targets of the lyophilized antibody panel.

Example 148 is the method of example(s) 147, further comprising automatically accessing a stored mapping of known targets for the lyophilized antibody panel, wherein the stored mapping associates known targets to associated mass channels.

Example 149 is the method of example(s) 136-148, further comprising: labelling the sample using a sample barcoding reagent, wherein the sample barcoding reagent comprises a distinct combination of isotopes usable to distinguish the sample barcoding reagent from an additional sample barcoding reagent; providing an additional sample; labelling the additional sample using the additional sample barcoding reagent; performing an additional surface stain on additional cells of the additional sample using the lyophilized antibody panel; mixing the assay barcoding reagent with the additional sample and removing unbound antibodies; and mixing the sample with the additional sample before interrogating the sample, wherein interrogating the sample comprises interrogating an admixture of the sample and the additional sample.

Example 150 is the method of example(s) 149, wherein mixing the sample with the additional sample occurs prior to performing the surface stain.

Example 151 is the method of example(s) 149 or 150, wherein the assay barcoding reagents are functionalized to bind to the sample barcoding reagents, wherein labelling the sample using the sample barcoding reagent comprises binding the sample barcoding reagents to a first portion of the assay barcoding reagents such that a second portion of the assay barcoding reagents is free of the sample barcoding reagents, wherein mixing the assay barcoding reagent with the additional sample comprises mixing the second portion of the assay barcoding reagents with the additional sample, and wherein mixing the sample with the additional sample occurs after mixing the assay barcoding reagents with the additional sample.

Example 152 is the method of example(s) 120-151, wherein interrogating the sample comprises obtaining data associated with the sample using an elemental analysis device, wherein the method further comprises automatically analyzing the data.

Example 153 is the method of example(s) 152, wherein automatically analyzing the data comprises applying a cleanup model to the data, wherein applying the cleanup model comprises accessing Gaussian measurements generated by the elemental analysis device associated with ionization of the sample.

Example 154 is the method of example(s) 152 or 153, wherein automatically analyzing the data comprises: accessing an element tag designation model, wherein the element tag designation model includes information associating each of the distinct element tags of the lyophilized antibody panel with a cell type; identifying presence information for the distinct element tags of the lyophilized antibody panel; and determining, for each cell of the sample, the cell type using the identified presence information for the distinct element tags and the element tag designation model.

Example 155 is a barcoding kit for elemental analysis, comprising a plurality of sample barcodes for labelling a plurality of samples, wherein each of the sample barcodes comprises a distinct combination of isotopes distinguishable by elemental analysis, and wherein each of the sample barcodes is stored in a distinct container; and a set of biomolecules bindable to the plurality of samples, wherein the set of biomolecules comprises the plurality of sample barcodes or is functionalized to bind to the plurality of sample barcodes.

Example 156 is the barcoding kit of example(s) 155, wherein each of the set of biomolecules comprises unique ones of the plurality of sample barcodes.

Example 157 is the barcoding kit of example(s) 155 or 156, wherein each of the set of biomolecules is functionalized to bind to the plurality of sample barcodes, and wherein the set of biomolecules is stored separately from the plurality of sample barcodes.

Example 158 is the barcoding kit of example(s) 155-157, wherein the set of biomolecules comprises a plurality of beads.

Example 159 is the barcoding kit of example(s) 158, wherein each bead comprises an external surface functionalized to bind to the plurality of sample barcodes.

Example 160 is the barcoding kit of example(s) 159, wherein each bead comprises an assay barcode within an interior of the bead, wherein each assay barcode comprises an additional combination of isotopes that is distinguishable from the distinct combinations of isotopes of the sample barcodes by elemental analysis.

Example 161 is a method, comprising: providing a plurality of samples comprising a first sample and a second sample; providing plurality of sample barcodes comprising a first sample barcode and a second sample barcode, wherein each of the sample barcodes comprises a distinct combination of isotopes distinguishable by elemental analysis; providing a plurality of biomolecules bindable to the plurality of samples, wherein each biomolecule comprises one of the plurality of sample barcodes or is functionalized to bind to the plurality of sample barcodes, and wherein the plurality of biomolecules comprises a first biomolecule and a second biomolecule; mixing the first biomolecule with the first sample; mixing the second biomolecule with the second sample; removing any unbound biomolecules; interrogating the plurality of samples using elemental analysis to obtain elemental data; detecting a presence of each distinct combination of isotopes in the elemental data; and associating the elemental data with one of the plurality of samples using the detected presence of each distinct combination of isotopes.

Example 162 is the method of example(s) 161, further comprising: mixing the first sample barcode with the mixture comprising the first biomolecule and the first sample; and mixing the second sample barcode with the mixture comprising the second biomolecule and the second sample.

Example 163 is the method of example(s) 161, further comprising: mixing the first sample barcode with the first biomolecule prior to mixing the first biomolecule with the first sample; and mixing the second sample barcode with the second biomolecule prior to mixing the second biomolecule with the second sample.

Example 164 is the method of example(s) 161-163, further comprising pooling the first sample and the second sample prior to interrogating the plurality of samples.

Example 165 is the method of example(s) 161-164, wherein the plurality of biomolecules comprises a plurality of beads.

Example 166 is the method of example(s) 165, wherein each bead comprises an external surface functionalized to bind to the plurality of sample barcodes.

Example 167 is the method of example(s) 166, wherein each bead comprises an assay barcode within an interior of the bead, wherein each assay barcode comprises an additional combination of isotopes that is distinguishable from the distinct combinations of isotopes of the sample barcodes by elemental analysis.

What is claimed is:

1. A panel for elemental analysis, comprising:
a plurality of conjugated antibodies, wherein each of the plurality of conjugated antibodies is tagged with a distinct element tag, wherein each distinct element tag is distinguishable based on its isotopic composition, and wherein the plurality of conjugated antibodies is in a lyophilized mixture.

2. The panel of claim 1, wherein the plurality of conjugated antibodies includes two or more antibodies from the list comprising Cd45, CD45RA, CD45RO, Cd123, CD4, CD8a, CD11C, CD57, CXCR3, CD185, CD38, CD56, CD3, CD20, CD66b, HLA-DR, IgD, CD27, CD28, CD127, CD19, CD16, CD161, CD194, CD25, CD294, CD197, CD14, CCR6, and TCR δγ.

3. The panel of claim 1, wherein a majority of the conjugated antibodies are specific to a cell type in human peripheral blood.

4. The panel of claim 1, wherein a majority of the conjugated antibodies are specific to a cell surface marker.

5. The panel of claim 1, wherein the plurality of conjugated antibodies comprises ten or more conjugated antibodies in the lyophilized mixture.

6. The panel of claim 1, wherein each distinct element tag comprises a plurality of elemental atoms of one isotope.

7. The panel of claim 1, wherein at least two conjugated antibodies of the plurality of conjugated antibodies are tagged with distinct element tags having different isotopes of a single element.

8. The panel of claim 1, further comprising a biomolecule coupled to an additional element tag, wherein the biomolecule is not an antibody, and wherein the additional element tag is distinguishable from each distinct element tag based on its isotopic composition.

9. The panel of claim 1, further comprising a non-antibody metal-containing moiety comprising a metal isotope that is distinguishable from each distinct element tag based on its isotopic composition.

10. The panel of claim 1, wherein each distinct element tag comprises a metal element having an atomic mass greater than 80 amu.

11. The panel of claim 1, wherein each distinct element tag comprises a chelated metal.

12. The panel of claim 1, wherein each distinct element tag comprises an element that is not endogenous to human peripheral blood.

13. The panel of claim 1, wherein the panel has a moisture content that is at or less than 5% by weight.

14. The panel of claim 1, wherein the panel has a moisture content that is at or less than 3% by weight.

15. The panel of claim 1, wherein the panel has a moisture content that is at or less than 1% by weight.

16. The panel of claim 1, wherein the panel has a moisture content that is at or between 0.05 and 1% by weight.

17. The panel of claim 1, further comprising a lyophilized intercalator, wherein the lyophilized intercalator is included in the lyophilized mixture.

18. The panel of claim 1, further comprising a lyophilized calibration material, wherein the lyophilized calibration material comprises known quantities of one or more known isotope(s), and wherein the lyophilized calibration material is included in the lyophilized mixture.

19. The panel of claim 1, further comprising a supplemental reagent, wherein the supplemental reagent is usable for conducting an assay using the plurality of conjugated antibodies, wherein the supplemental reagent is lyophilized, and wherein the supplemental reagent is included in the lyophilized mixture.

20. An assay kit for use with elemental analysis, comprising:
a hermetically sealed container; and
the panel of claim 1, wherein the lyophilized mixture of the panel is stored within the hermetically sealed container.

21. The assay kit of claim 20, wherein the hermetically sealed container includes an internal atmosphere of an inert gas or dry air.

22. The assay kit of claim 20, wherein the plurality of conjugated antibodies includes a first antibody specific to a cell surface marker and second antibody specific to an intracellular target.

23. The assay kit of claim 20, further comprising:
an additional hermetically sealed container; and
an additional antibody panel comprising at least one additional conjugated antibody tagged with an additional distinct element tag that is distinguishable from each distinct element tag based on its isotopic composition, wherein the additional conjugated antibody is lyophilized and stored within the additional hermetically sealed container.

24. The assay kit of claim 23, wherein the plurality of conjugated antibodies includes antibodies specific to one or more cell surface markers, and wherein the additional conjugated antibody is specific to an intracellular target.

25. The assay kit of claim 20, further comprising an intercalator comprising an additional distinct element tag that is distinguishable from each distinct element tag based on its isotopic composition.

26. The assay kit of claim 20, further comprising a plurality of sample barcoding reagents for labelling a plurality of samples, wherein each of the plurality of sample barcoding reagents comprises a distinct combination of isotopes.

27. The assay kit of claim 26, further comprising a plurality of containers, wherein each of the plurality of sample barcoding reagents is contained within different containers of the plurality of containers.

28. The assay kit of claim 26, wherein each of the plurality of sample barcoding reagents binds to a majority of cells in a sample.

29. The assay kit of claim 26, wherein each of the plurality of sample barcoding reagents comprise element tags functionalized to covalently bind on or within cells of a sample.

30. The assay kit of claim 26, wherein each of the plurality of sample barcoding reagents comprises sample barcoding antibodies that specifically bind a target present across a majority of cells in a sample or that together binds multiple targets across a majority of cells in the sample.

31. The assay kit of claim 30, wherein each of the sample barcoding antibodies specifically binds one or more of CD45, CD298 and b2m.

32. The assay kit of claim 30, wherein the element tags of the sample barcoding antibodies provide a weaker signal than the element tags of the majority of other antibodies in the panel when analyzed using an elemental analyzer.

33. The assay kit of claim 26, wherein the distinct combination of isotopes comprises cadmium.

34. The assay kit of claim 26, wherein the distinct combination of isotopes comprises platinum in cisplatin.

35. The assay kit of claim 26, wherein each of the plurality of sample barcoding reagents comprises a set of sample barcoding antibodies, wherein each sample barcoding antibody comprises all of the isotopes of the distinct combination of isotopes.

36. The assay kit of claim 26, wherein each of the plurality of sample barcoding reagents is capable of barcoding live cells, and wherein each of the plurality of sample barcoding reagents is non-toxic to the live cells.

37. The assay kit of claim 20, further comprising assay barcoding reagents comprising additional antibodies for detecting different analytes, wherein each assay barcoding reagent comprises a distinct combination of isotopes.

38. The assay kit of claim 37, wherein each assay barcoding reagent is an assay barcoding bead comprising the distinct combination of isotopes.

39. The assay kit of claim 38, wherein the assay barcoding reagents are included in the lyophilized mixture of the panel.

40. The assay kit of claim 38, wherein each assay barcoding bead comprises a unique combination of isotopes present within an interior of the assay barcoding bead.

41. The assay kit of claim 37, wherein the assay barcoding reagents comprise at least ten assay barcoding reagents for barcoding at least ten different analytes, and wherein the assay barcoding reagents are provided in admixture.

42. The assay kit of claim 37, wherein the different analytes are free analytes in human peripheral blood.

43. The assay kit of claim 37, further comprising a combination of reporter antibodies that specifically bind the different analytes, wherein each reporter antibody comprises an element tag detectable by elemental analysis.

44. The assay kit of claim 43, wherein each of the element tags of the combination of reporter antibodies comprises an isotopically identical element detectable by elemental analysis.

45. The assay kit of claim 37, wherein each assay barcoding reagent is functionalized to attach to a sample barcode comprising a sample barcoding composition of isotopes.

46. The assay kit of claim 45, further comprising the sample barcode comprising the sample barcoding composition of isotopes.

47. The assay kit of claim 45, wherein the sample barcode can bind to cells of a sample stained with the lyophilized panel.

48. The assay kit of claim 20, further comprising barcoding reagents, wherein each barcoding reagent comprises an assay barcoding composition of isotopes and a sample barcoding composition of isotopes, wherein each distinct assay barcoding composition of isotopes is associated with a distinct analyte, and wherein each distinct sample barcoding composition of isotopes can be associated with a distinct sample.

49. The assay kit of claim 48, wherein each barcoding reagent is a bead, and wherein the sample barcoding composition of isotopes is located within an interior of the bead.

50. The assay kit of claim 48, wherein each barcoding reagent is a bead, and wherein the sample barcoding composition of isotopes is located on a surface of the bead.

51. The assay kit of claim 20, further comprising an anticoagulant.

52. The assay kit of claim 20, further comprising a calibration material, wherein the calibration material comprises a known quantity of a known isotope.

53. An assay kit for use with elemental analysis, comprising:
a plurality of hermetically sealed containers; and
the panel of claim 1, wherein the lyophilized mixture of the panel is distributed across the plurality of hermetically sealed containers.

54. The assay kit of claim 53, further comprising a plurality of sample barcoding reagents for labelling a plurality of samples, wherein each of the plurality of sample barcoding reagents comprises a distinct combination of isotopes, and wherein each of the plurality of sample barcoding reagents is contained within different containers of the plurality of hermetically sealed containers.

55. A barcoding system, comprising:
a barcoding reagent comprising an assay barcode, wherein the assay barcode comprises a composition of isotopes associated with a target analyte, wherein the composition of isotopes is distinguishable through elemental analysis, wherein the barcoding reagent comprises one of a plurality of sample barcodes or is functionalized to bind to at least one of the plurality of sample barcodes, wherein each sample barcode of the plurality of sample barcodes comprises a unique additional composition of isotopes distinguishable from the assay barcode composition of isotopes through elemental analysis, wherein each sample barcode of the plurality of sample barcodes can be associated with a distinct sample.

56. The system of claim 55, wherein the barcoding reagent is a bead, and wherein the sample barcode is present in the interior of the bead.

57. The system of claim 55, wherein the barcoding reagent is a bead, and wherein a surface of the bead is functionalized to bind to the plurality of sample barcodes.

58. The system of claim 55, wherein the barcoding reagent is a bead, and wherein a surface of the bead comprises the one of the plurality of sample barcodes.

59. The system of claim 55, wherein the barcoding reagent is functionalized to bind to the plurality of sample barcodes, and wherein the system further comprises each sample barcode of the plurality of sample barcodes in separate containers.

60. The system of claim 55, further comprising a sample barcoding reagent comprising at least one of the plurality of sample barcodes, wherein the sample barcoding reagent can bind both the barcoding reagent and cells of a sample.

61. The system of claim 55, wherein the barcoding reagent is a bead, and wherein the assay barcode is present in the interior of the bead, and wherein the interior of the bead comprises a solid metal core, metal chelating polymer interior, nanocomposite interior, or hybrid interior.

62. The system of claim 61, wherein the bead has a solid metal core and a polymer surface.

63. The system of claim 62, wherein the polymer surface is bound to an antibody that binds to the target analyte.

64. The system of claim 63, wherein the target analyte is a free analyte present in blood.

65. The system of claim 55, further comprising a reporter antibody that specifically binds the target analyte and comprises an elemental tag or a combination of high and low intensity element tag(s).

66. The system of claim 55, wherein the assay barcoding reagents comprise at least ten assay barcoding reagents for barcoding at least ten different analytes, and wherein a plurality of separate mixtures of the assay barcoding reagents each comprise an isotopically distinguishable sample barcode.

67. The system of claim 66, further comprising reporter biomolecules that specifically bind the target analytes of the assay barcoding reagents, wherein the reporter biomolecules comprise each comprise an affinity reagent or oligonucleotides, and wherein each reporter biomolecule comprises an element tag or a combination of a high and low signal element tag.

68. The system of claim 67, wherein at least some of the reporter biomolecules that specifically bind different target analytes comprise the same element tag(s).

69. A method, comprising:
providing a plurality of antibodies;
conjugating each of the plurality of antibodies with a distinct element tag, wherein each distinct element tag is distinguishable based on its isotopic composition, and wherein each of the plurality of antibodies is distinguishable by its distinct element tag;
mixing the plurality of conjugated antibodies together into an admixture; and
lyophilizing the admixture.

70. The method of claim 69, further comprising spin filtering the plurality of conjugated antibodies.

71. The method of claim 69, further comprising:
selecting an interrogation scheme for interrogating a sample; and
selecting the plurality of antibodies based on the selected interrogation scheme.

72. The method of claim 69, wherein providing the plurality of antibodies includes providing two or more antibodies from the list including Cd45, CD45RA, CD45RO, Cd123, CD4, CD8a, CD11C, CD57, CXCR3, CD185, CD38, CD56, CD3, CD20, CD66b, HLA-DR, IgD, CD27, CD28, CD127, CD19, CD16, CD161, CD194, CD25, CD294, CD197, CD14, CCR6, and TCR δγ.

73. The method of claim 69, wherein each of the plurality of antibodies is specific to a cell type in human peripheral blood.

74. The method of claim 69, wherein each of the plurality of antibodies is specific to a cell surface marker.

75. The method of claim 69, wherein mixing the plurality of conjugated antibodies together comprises mixing together ten or more antibodies.

76. The method of claim 69, wherein each distinct element tag comprises a plurality of elemental atoms of one isotope.

77. The method of claim 69, wherein at least two of the distinct element tags have different isotopes of a single element.

78. The method of claim 69, further comprising providing a biomolecule comprising an additional element tag, wherein the additional element tag is distinguishable from each distinct element tag based on its isotopic composition, wherein the biomolecule is not an antibody, and wherein mixing the plurality of conjugated antibodies together further comprises mixing the biomolecule with the plurality of conjugated antibodies.

79. The method of claim 69, wherein each distinct element tag comprises a metal element having an atomic mass greater than 80 amu.

80. The method of claim 69, wherein each distinct element tag comprises a chelated metal.

81. The method of claim 69, wherein each distinct element tag comprises an element that is not endogenous to human peripheral blood.

82. The method of claim 69, wherein lyophilizing the admixture comprises reducing the moisture content to at or less than 5% by weight.

83. The method of claim 69, further comprising mixing an intercalator into the admixture before lyophilizing the admixture, wherein the intercalator comprises an additional element tag that is distinguishable from each distinct element tag based on its isotopic composition.

84. The method of claim 69, further comprising mixing a calibration material into the admixture before lyophilizing the admixture, wherein the calibration material comprises a known quantity of a known isotope.

85. The method of claim 69, further comprising mixing a supplemental reagent into the admixture before lyophilizing the admixture, wherein the supplemental reagent is usable to facilitate conducting an assay using the plurality of conjugated antibodies.

86. The method of claim 69, wherein lyophilizing the mixture further comprises storing the lyophilized mixture in a hermetically sealed container with an internal atmosphere of an inert gas or dry air.

87. The method of claim 69, wherein the plurality of antibodies includes a first antibody specific to a cell surface marker and second antibody specific to an intracellular target.

88. The method of claim 69, further comprising:
providing at least one additional antibody;
conjugating the at least one additional antibody with at least one additional distinct element tag that is distinguishable from each distinct element tag based on its isotopic composition;
lyophilizing the at least one additional antibody; and
storing the lyophilized at least one additional antibody separately from the lyophilized admixture.

89. The method of claim 88, wherein the plurality of antibodies includes antibodies specific to one or more cell surface markers, and wherein the additional antibody is specific to an intracellular target.

90. The method of claim 69, further comprising providing a plurality of sample barcoding reagents for labelling a plurality of samples, wherein each of the plurality of sample barcoding reagents comprises a distinct combination of isotopes.

91. The method of claim 90, further comprising:
providing a plurality of containers; and
storing each of the plurality of sample barcoding reagents within different containers of the plurality of containers.

92. The method of claim 90, wherein each of the plurality of sample barcoding reagents binds to a majority of cells in a sample.

93. The method of claim 90, wherein each of the plurality of sample barcoding reagents comprise element tags functionalized to covalently or otherwise permanently bind on or within cells of a sample.

94. The method of claim 90, wherein each of the plurality of sample barcoding reagents comprises a sample barcoding antibody that specifically binds a target present across a majority of cells in a sample.

95. The method of claim 94, wherein each of the sample barcoding antibodies specifically binds one or more of CD45, CD298, and b2m.

96. The method of claim 94, wherein each of the sample barcoding antibodies specifically binds a target present in a sample selected to result in a mass signal of a single sample barcoding antibody being weaker than a mass signal of a majority of the plurality of conjugated antibodies.

97. The method of claim 90, wherein the distinct combination of isotopes comprises cadmium.

98. The method of claim 90, wherein the distinct combination of isotopes comprises platinum in cisplatin.

99. The method of claim 90, wherein each of the plurality of sample barcoding reagents comprises a set of sample barcoding antibodies, wherein each sample barcoding antibody comprises all of the isotopes of the distinct combination of isotopes.

100. The method of claim 90, wherein each of the plurality of sample barcoding reagents is capable of barcoding live cells, and wherein each of the plurality of sample barcoding reagents is non-toxic to the live cells.

101. The method of claim 69, further comprising:
providing assay barcoding reagents comprising additional antibodies for detecting different analytes, wherein each assay barcoding reagent comprises a distinct combination of isotopes.

102. The method of claim 101, wherein each assay barcoding reagent is an assay barcoding bead comprising a distinct combination of isotopes.

103. The method of claim 102, wherein each assay barcoding bead comprises a unique combination of isotopes present within an interior of the assay barcoding bead.

104. The method of claim 101, wherein the assay barcoding reagents comprise at least ten assay barcoding reagents for barcoding at least ten different analytes, and wherein the assay barcoding reagents are provided in admixture.

105. The method of claim 101, wherein the different analytes are free analytes in human peripheral blood.

106. The method of claim 101, further comprising providing a combination of reporter antibodies that specifically bind the different analytes, wherein each reporter antibody comprises an element tag detectable by elemental analysis.

107. The method of claim 106, wherein each of the element tags of the combination of reporter antibodies comprises an isotopically identical element detectable by elemental analysis.

108. The method of claim 101, further comprising functionalizing each assay barcoding reagent to attach to a sample barcode comprising a sample barcoding composition of isotopes.

109. The method of claim 108, wherein the sample barcode binds cells of a sample to be assayed by the lyophilized admixture.

110. The method of claim 108, further comprising providing the sample barcode comprising the sample barcoding composition of isotopes.

111. The method of claim 69, further comprising providing barcoding reagents, wherein each barcoding reagent comprises an assay barcoding composition of isotopes and a sample barcoding composition of isotopes, wherein each distinct assay barcoding composition of isotopes is associated with a distinct analyte, and wherein each distinct sample barcoding composition of isotopes is associated with a distinct sample.

112. The method of claim 111, wherein each barcoding reagent is a bead, and wherein the sample barcoding composition of isotopes is located within an interior of the bead.

113. The method of claim 111, wherein each barcoding reagent is a bead, and wherein the sample barcoding composition of isotopes is located on a surface of the bead.

114. The method of claim 69, further comprising providing an anticoagulant.

115. The method of claim 69, further comprising titrating and diluting each of the plurality of conjugated antibodies to a predetermined concentration prior to mixing the plurality of conjugated antibodies.

116. The method of claim 69, further comprising mixing the plurality of conjugated antibodies with an excipient prior to lyophilizing the admixture.

117. The method of claim 116, wherein the excipient comprises a sugar and bovine serum albumin.

118. The method of claim 116, further comprising mixing the plurality of conjugated antibodies with a viability stain prior to lyophilizing the admixture.

119. The method of claim 118, wherein the viability stain is a rhodium intercalator.

120. A method, comprising:
preparing a sample;
providing a lyophilized antibody panel comprising a plurality of conjugated antibodies, wherein each of the plurality of conjugated antibodies is tagged with a distinct element tag, and wherein each distinct element tag is distinguishable based on its isotopic composition;
performing a surface stain on cells of the sample using the lyophilized antibody panel; and
interrogating the sample using elemental analysis to detect a presence of the distinct element tags.

121. The method of claim 120, wherein interrogating the sample using elemental analysis comprises processing the sample on an inductively coupled plasma mass spectrometer to detect a presence of the distinct element tags of the lyophilized antibody panel.

122. The method of claim 120, further comprising staining the sample of cells with a viability stain.

123. The method of claim 120, wherein the viability stain is provided as part of the lyophilized antibody panel.

124. The method of claim 123, wherein the viability stain is rhodium.

125. The method of claim 120, further comprising performing a FcR block on the sample of cells.

126. The method of claim 120, further comprising fixing the sample after performing the surface stain.

127. The method of claim 120, further comprising staining the sample with an intercalator, wherein the intercalator comprises an additional distinct element tag that is distinguishable from each distinct element tag based on its isotopic composition.

128. The method of claim 127, wherein staining the sample with the intercalator occurs after permeabilizing the sample.

129. The method of claim 120, further comprising permeabilizing the sample and performing an intracellular stain on the sample of cells using at least one additional antibody, wherein the at least one additional antibody is tagged with an additional distinct element tag that is distinguishable from each distinct element tag based on its isotopic composition.

130. The method of claim 120, wherein preparing the sample comprises collecting whole blood.

131. The method of claim 130, wherein preparing the sample comprises isolating peripheral blood mononuclear cells from the whole blood.

132. The method of claim 120, further comprising labelling the sample using a sample barcoding reagent, wherein the sample barcoding reagent comprises a distinct combination of isotopes usable to distinguish the sample barcoding reagent from an additional sample barcoding reagent.

133. The method of claim 132, wherein interrogating the sample comprises acquiring data through elemental analysis, identifying the sample barcoding reagent by the distinct combination of isotopes in the acquired data, and associating the acquired data with the sample.

134. The method of claim 133, wherein interrogating the sample further comprises mixing the sample with an additional sample prior to acquiring data through elemental analysis.

135. The method of claim 120, wherein performing a surface stain comprises:
adding a suspension of the cells of the sample to the lyophilized antibody panel or a resuspension of the lyophilized antibody panel; and
removing unbound antibodies.

136. The method of claim 120, further comprising:
providing assay barcoding reagents comprising additional antibodies for detecting different analytes, wherein each assay barcoding reagent comprises a distinct combination of isotopes; and
mixing the assay barcoding reagent with the sample and removing unbound antibodies before interrogating the sample.

137. The method of claim 136, wherein the sample comprises blood plasma, and wherein the different analytes are free analytes within the blood plasma.

138. The method of claim 136, wherein providing the assay barcoding reagents and providing the lyophilized antibody panel occur by providing an admixture of the lyophilized antibody panel and the assay barcoding reagents.

139. The method of claim 136, wherein preparing the sample comprises collecting whole blood.

140. The method of claim 139, wherein preparing the sample further comprises isolating peripheral blood mononuclear cells and blood plasma, wherein performing the surface stain comprises mixing the lyophilized antibody panel with the peripheral blood mononuclear cells and removing unbound antibodies; and wherein mixing the assay barcoding reagents with the sample comprises mixing the assay barcoding reagents with the blood plasma and removing unbound antibodies.

141. The method of claim 120, wherein interrogating the sample further comprises automatically identifying cell viability.

142. The method of claim 120, wherein interrogating the sample further comprises automatically identifying cell populations.

143. The method of claim 142, wherein interrogating the sample further comprises identifying characteristics of automatically identified cell populations.

144. The method of claim 143, wherein interrogating the sample further comprises comparing the identified characteristics across the identified cell populations or comparing the identified characteristics associated with one of the identified cell populations with additional characteristics identified associated with the same one of the identified cell populations from an additional sample.

145. The method of claim 143, wherein identifying characteristics of automatically identified cell populations comprises determining an abundance of one or more targets on or in cells of the identified cell populations.

146. The method of claim 143, wherein the identified characteristics comprise a percentage of cells in the cell populations.

147. The method of claim 120, wherein interrogating the sample further comprise generating at least one of a histogram, a 2D dot plots, and a tSNE graph based on known targets of the lyophilized antibody panel.

148. The method of claim 147, further comprising automatically accessing a stored mapping of known targets for the lyophilized antibody panel, wherein the stored mapping associates known targets to associated mass channels.

149. The method of claim 136, further comprising:
labelling the sample using a sample barcoding reagent, wherein the sample barcoding reagent comprises a distinct combination of isotopes usable to distinguish the sample barcoding reagent from an additional sample barcoding reagent;
providing an additional sample;
labelling the additional sample using the additional sample barcoding reagent;
performing an additional surface stain on additional cells of the additional sample using the lyophilized antibody panel;
mixing the assay barcoding reagent with the additional sample and removing unbound antibodies; and
mixing the sample with the additional sample before interrogating the sample, wherein interrogating the sample comprises interrogating an admixture of the sample and the additional sample.

150. The method of claim 149, wherein mixing the sample with the additional sample occurs prior to performing the surface stain.

151. The method of claim 149, wherein the assay barcoding reagents are functionalized to bind to the sample barcoding reagents, wherein labelling the sample using the sample barcoding reagent comprises binding the sample barcoding reagents to a first portion of the assay barcoding reagents such that a second portion of the assay barcoding reagents is free of the sample barcoding reagents, wherein mixing the assay barcoding reagent with the additional sample comprises mixing the second portion of the assay barcoding reagents with the additional sample, and wherein mixing the sample with the additional sample occurs after mixing the assay barcoding reagents with the additional sample.

152. The method of claim 120, wherein interrogating the sample comprises obtaining data associated with the sample using an elemental analysis device, wherein the method further comprises automatically analyzing the data.

153. The method of claim 152, wherein automatically analyzing the data comprises applying a cleanup model to the data, wherein applying the cleanup model comprises accessing Gaussian measurements generated by the elemental analysis device associated with ionization of the sample.

154. The method of claim 152, wherein automatically analyzing the data comprises:
accessing an element tag designation model, wherein the element tag designation model includes information associating each of the distinct element tags of the lyophilized antibody panel with a cell type;
identifying presence information for the distinct element tags of the lyophilized antibody panel; and
determining, for each cell of the sample, the cell type using the identified presence information for the distinct element tags and the element tag designation model.

155. A barcoding kit for elemental analysis, comprising
a plurality of sample barcodes for labelling a plurality of samples, wherein each of the sample barcodes comprises a distinct combination of isotopes distinguishable by elemental analysis, and wherein each of the sample barcodes is stored in a distinct container; and
a set of biomolecules bindable to the plurality of samples, wherein the set of biomolecules comprises the plurality of sample barcodes or is functionalized to bind to the plurality of sample barcodes.

156. The barcoding kit of claim 155, wherein each of the set of biomolecules comprises unique ones of the plurality of sample barcodes.

157. The barcoding kit of claim 155, wherein each of the set of biomolecules is functionalized to bind to the plurality of sample barcodes, and wherein the set of biomolecules is stored separately from the plurality of sample barcodes.

158. The barcoding kit of claim 155, wherein the set of biomolecules comprises a plurality of beads.

159. The barcoding kit of claim 158, wherein each bead comprises an external surface functionalized to bind to the plurality of sample barcodes.

160. The barcoding kit of claim 159, wherein each bead comprises an assay barcode within an interior of the bead, wherein each assay barcode comprises an additional combination of isotopes that is distinguishable from the distinct combinations of isotopes of the sample barcodes by elemental analysis.

161. A method, comprising:
providing a plurality of samples comprising a first sample and a second sample;
providing plurality of sample barcodes comprising a first sample barcode and a second sample barcode, wherein each of the sample barcodes comprises a distinct combination of isotopes distinguishable by elemental analysis;
providing a plurality of biomolecules bindable to the plurality of samples, wherein each biomolecule comprises one of the plurality of sample barcodes or is functionalized to bind to the plurality of sample barcodes, and wherein the plurality of biomolecules comprises a first biomolecule and a second biomolecule;
mixing the first biomolecule with the first sample;
mixing the second biomolecule with the second sample;
removing any unbound biomolecules;
interrogating the plurality of samples using elemental analysis to obtain elemental data;
detecting a presence of each distinct combination of isotopes in the elemental data; and
associating the elemental data with one of the plurality of samples using the detected presence of each distinct combination of isotopes.

162. The method of claim 161, further comprising:
mixing the first sample barcode with the mixture comprising the first biomolecule and the first sample; and
mixing the second sample barcode with the mixture comprising the second biomolecule and the second sample.

163. The method of claim 161, further comprising:
mixing the first sample barcode with the first biomolecule prior to mixing the first biomolecule with the first sample; and
mixing the second sample barcode with the second biomolecule prior to mixing the second biomolecule with the second sample.

164. The method of claim 161, further comprising pooling the first sample and the second sample prior to interrogating the plurality of samples.

165. The method of claim 161, wherein the plurality of biomolecules comprises a plurality of beads.

166. The method of claim 165, wherein each bead comprises an external surface functionalized to bind to the plurality of sample barcodes.

167. The method of claim 166, wherein each bead comprises an assay barcode within an interior of the bead, wherein each assay barcode comprises an additional combination of isotopes that is distinguishable from the distinct combinations of isotopes of the sample barcodes by elemental analysis.

168. An assay barcoded kit comprising:
a plurality of reporters for detecting the presence of target analytes; and
a plurality of individual assay beads that comprise:
a capture biomolecule that specifically binds a target and is bound to the bead surface; and
an assay barcode comprising a composition of isotopes or elements associated with the capture biomolecule, wherein the assay barcode is distinguishable by mass spectrometry from the assay barcode of assay beads with a capture biomolecule that binds to a different target.

169. The kit of claim 168, wherein the plurality of individual assay beads are provided in mixture

170. The assay barcoded kit of claim 168 or 169, wherein the reporter comprises an antibody or oligonucleotide conjugated to a nanoparticle mass tag.

171. The assay barcoded kit of any one of claims 168 to 170, the reporter comprises a reporter oligonucleotide that directly hybridizes to the target analyte.

172. The assay barcoded kit of any one of claims 168 to 170, wherein the reporter comprises a mass tagged oligonucleotide that hybridizes to an intermediate reporter oligonucleotide that hybridizes, directly or indirectly, to the target analyte.

173. The assay barcoded kit of any one of claims 168 to 172, wherein the target is the target analyte.

174. The assay barcoded kit of any one of claims 168 to 172, wherein the target comprises an intermediate capture oligonucleotide comprising a complementary sequence to an oligonucleotide of the capture biomolecule, and wherein the target further comprises an assay biomolecule that binds to a target analyte of a sample.

175. The assay barcoded kit of claim 175, wherein the intermediate capture biomolecule further comprises a mass tag that provides a sample barcode.

176. The assay barcoded kit of any one of claims 168 to 175, wherein a reporter that binds the target analyte comprises an oligonucleotide sequence that hybridizes, directly or indirectly, to a mass tagged oligonucleotide.

177. The assay barcoded kit of any one of claims 168 to 176, wherein the reporters for different target analytes comprise the same mass tag

178. The assay barcoded kit of any one of claims 168 to 177, comprising a mixture of reporters for different target analytes.

179. The assay barcoded kit of any one of claims 168 to 178, the assay barcode beads comprises one of a plurality of sample barcodes or is functionalized to bind to the plurality of sample barcodes, wherein each sample barcode of the plurality of sample barcodes comprises a unique additional composition of isotopes distinguishable from the composition of isotopes through elemental analysis, wherein each sample barcode of the plurality of sample barcodes is associated with a distinct sample

180. The assay barcoded kit of any one of claims 168 to 178, wherein reporters comprise a system for signal amplification.

181. The assay barcoded kit of claim 180, wherein the signal amplification is by a hybridization scheme.

182. A method of analysis of target analyte with assay barcoded beads of any one of kit claims 168 to 181, comprising:
incubating the assay barcoded beads with target analyte in a sample such that target analyte is bound to assay barcoded beads;
incubating the assay barcoded beads with the plurality of reporters;
detecting, by mass spectrometry, the assay barcode alongside the mass tag of a reporter on individual beads.

183. A method of elemental analysis comprising:
separating cells of a sample into a plurality of partitions;
staining cells with a shared panel of mass tagged antibodies;
staining cells of separate partitions with distinct panels of mass tagged biomolecules, wherein individual distinct panels comprise biomolecules that are not in other distinct panels but that are tagged with a mass tags present in the other distinct panels; and
interrogating the sample using elemental analysis to detect a presence of the distinct mass tags on individual cells.

184. The method of claim 183, wherein the shared panel is conserved across two or more partitions.

185. The method of claim 183 or 184, wherein the shared panel detects positively expressed surface targets that distinguish parent populations, wherein the parent populations together cover the majority of immune cells.

186. The method of any one of claims 183 to 185, wherein one or more individual distinct panels each detect positively expressed surface targets that distinguish sub-populations within one of the parent populations, but do not distinguish sub-populations for the majority of immune cells.

187. The method of any one of claims 183 to 185, further comprising labeling partitioned cells with a panel barcode that identifies the distinct panel.

188. The method of any one of claims 183 to 186, further comprising classifying individual interrogated cell into cell populations based on the shared panel and its distinct panel.

189. The method of claim 188, wherein classification is automated by software trained on the shared panel and distinct panels.

190. The method of claim further comprising integrating interrogated cell populations identified based on different distinct panels into the same data set based on the shared panel.

191. A kit for performing any one of claims 183 to 190.

192. A kit for elemental analysis, comprising:
a shared panel comprising a plurality of antibodies each conjugated to a distinct mass tag, wherein each distinct mass tag is distinguishable based on its isotopic composition, wherein the plurality of conjugated antibodies is in mixture; and
a plurality of distinct panels comprising mass tagged biomolecules, wherein the distinct panels comprise biomolecules that are different from one another but comprise overlapping mass tags.

193. A kit for cell segmentation, comprising:
a membrane stain comprising a plurality of antibodies to different cell surface targets, wherein the antibodies are conjugated to the same mass tag;
wherein the membrane stain does not comprise antibodies that stains targets in compartments other than the plasma membrane

194. The kit of claim 194, wherein the membrane stain comprises an antibody to a junction protein and an antibody to a non junction protein.

195. A method of using the kit of claim 193 or 194 to stain cells in a tissue section sample.

196. The method of claim 195, wherein the membrane stain is applied after other antibody stains.

197. The method of claim 195 or 196, further comprising interrogating the sample by imaging mass cytometry.

198. The method of claim 197, further comprising segmenting the cells based at least in part the membrane stain.

199. The method of claim 198, further comprising identifying populations of segmented cells based on detection of antibodies to cell surface markers, wherein the antibodies are conjugated to distinct mass tags.

200. The method of claim 198 or 199, wherein cell segmentation is automated by software.
